(19) United States
(12) Patent Application Publication
(10) Pub. No.: US 2022/0332695 A1
(43) Pub. Date: Oct. 20, 2022

(54) UREA DERIVATIVES AS CB1 ALLOSTERIC MODULATORS

(71) Applicant: RTI INTERNATIONAL, Research Triangle Park, NC (US)

(72) Inventors: Yanan ZHANG, Research Triangle Park, NC (US); Thuy T. NGUYEN, Research Triangle Park, NC (US)

(21) Appl. No.: 17/621,002

(22) PCT Filed: Jun. 25, 2020
(86) PCT No.: PCT/US2020/039644

§ 371 Date: Dec. 20, 2021

Related U.S. Application Data
(60) Provisional application No. 62/868,126, filed on Jun. 28, 2019.

(51) Int. Cl. C07D 333/36; C07C 275/30; A61P 25/30

(52) U.S. Cl. CPC C07D 333/36; C07C 275/30; A61P 25/30

(57) ABSTRACT

Heteroaryl and aliphatic analogs of diarylurea-based cannabinoid 1 receptor (CB1R) allosteric modulators are described. Exemplary analogs can provide improved potencies and pharmacokinetic properties. Methods of using the analogs to treat diseases mediated by CB1R, such as substance abuse and obesity, are described.

### CROSS-REFERENCE TO RELATED APPLICATIONS

This application claims benefit of U.S. Provisional Patent Application Ser. No. 62/868,126, filed Jun. 28, 2019, herein incorporated by reference in its entirety.

### GOVERNMENT INTEREST

This invention was made with government support from under Grant No. DA040693 awarded by the National Institutes of Health. The government has certain rights in the invention.

### TECHNICAL FIELD

The presently disclosed subject matter relates to urea-based cannabinoid 1 receptor (CB1R) allosteric modulator compounds, and to pharmaceutical compositions and uses thereof. Uses of the compounds include the modulation of CB1R activity and the treatment of diseases and conditions mediated by CB1R, such as obesity, drug abuse, alcohol addition, anxiety, depression, metabolic syndrome, stroke, hypotension, impaired fertility, cancer, inflammation, Parkinson's desease, paralytic ileus, and osteoporosis.

### BACKGROUND

According to the 2017 National Survey on Drug Use and Health, 18.7 million adults in the United States were affected with a substance abuse disorder. There are currently no FDA-approved medications for the treatment of craving to stimulants (e.g., cocain, methamphetamine) and cannabis (marijuana). There are available medications for relapse prevention of other addictive substances (e.g., opioids, tobacco, and alcohol). However, while these medications can be effective for the treatment of withdrawal symptoms, the long-term abstinence rate is still low. For example, even with several medications for smoking cessation, the one-year abstinence rate is only about 20%, compared to about 10% for placebo. Accordingly, there is an unmet demand for medications that alleviate substance craving on a long-term basis.

The cannabinoid 1 and cannabinoid 2 receptors (CB1R and CB2R, respectively) belong to the Class A Rhodopsin-like superfamily of G protein-coupled receptors (GPCRs). CB1R is one of the most abundantly expressed receptors in the brain. See Matsuda et al., Nature 1990, 346,561-564. CB1R plays a role in many physiological processes, such as pain, learning and memory, appetite and feeing behaviors, anxiety and depression. See Porter et al., Pharmacol. Ther. 2001, 90, 45-60; Harkany et al., Trends Pharmacol. Sci. 2007, 28, 83-92; and Kreitzer and Regehr, Curr. Opin. Neurobiol. 2002, 12, 324-330. As (−)-trans-Δ⁹-tetrahydrocannabinol (THC), the major phytocannabinoid found in marijuana, has been known for centuries to induce appetite and weight gains, as well as addiction, CB1R has been investigated to develop therapeutic interventions for obesity, metabolic disorders and substance abuse. See Van Gaal et al., Lancet 2005, 365, 1389-1397; Pi-Sunyer et al., JAMA 2006, 295, 761-775; Scheen et al., Lancet 2006, 368, 1660-1672;Rosenstock et al., Diabetes Care 2008, 31, 2169-2176; Despres et al., Arterioscler. Thromb. Vasc. Biol. 2009, 29, 416-423; Steinberg and Foulds, Vasc. Health Risk Manag. 2007, 3, 307-311; and Huestis et al., Psychopharmacology (Berl) 2007, 194, 505-515. Other potential uses of

CB1R antagonists/inverse agonists include the treatment of cancer, impaired fertility in women, stroke, hypotension, and intestinal hypomotility in paralytic ileus. See Pertwee and Thomas, “Therapeutic Applications for Agents that Act at CB1 and CB2Receptors,” in The Cannabinoid Receptors, Reggio, Ed., Humana Press: 2009, pp. 361-392; and Youssif et al., European Journal of Medicinal Chemistry 2019, 177, 1-11. Unfortunately, rimonabant (also known as SR₁₄₁₇₁₆A), the first CB1R inverse agonist/antagonist that received FDA approval for the treatment of obesity in 2006, was subsequently withdrawn due to adverse effects, including suicidal ideation.

Accordingly, there is an ongoing need for additional compounds that can modulate CB1 activity to treat substance addiction, and other conditions that can be modulated via CB1R. For example, there is an ongoing need for additional CB1R modulator compounds that have reduced side effects, improved pharmacokinetic properties (e.g., metabolic stability), and improved potencies.

### SUMMARY

In some embodiments, the presently disclosed subject matter provides a compound having a structure of Formula (I):

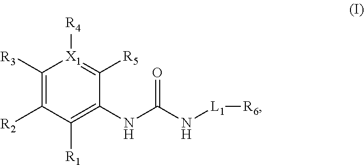

wherein: X₁ is —C— or —N—; each of R₁, R₂, R₃, and R₅ is independently selected from the group comprising H, alkyl, substituted alkyl, halo, haloalkyl, alkoxy, nitro, and cyano, or wherein R₂ and R₃ together form an alkylene group; R₄ is present or absent, and when present is selected from the group comprising H, alkyl, substituted alkyl, halo, haloalkyl, alkoxy, nitro, and cyano; L₁ is selected from the group comprising alkylene, substituted alkylene, cycloalkylene, substituted cycloalkylene, heterocycloalkylene, substituted arylene, heteroarylene, and substituted heteroarylene; and R₆ is selected from the group comprising aryl, substituted aryl, heteroaryl, substituted heteroaryl, alkylamino, dialkylamino, acylamino, N-heterocycle, and substituted N-heterocycle; or a pharmaceutically acceptable salt or solvate thereof. In some embodiments, X₁ is —C—.

In some embodiments, R₁, R₂, R₄, and R₅ are each H, and the compound of Formula (I) has a structure of Formula (Ia):

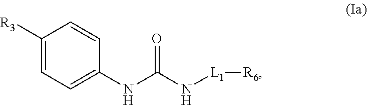

or a pharmaceutically acceptable salt or solvate thereof. In some embodiments, R₃ is Cl. In some embodiments, L₁ is selected from the group comprising thiophenylene, pyridinylene, thiazolylene, alkylene, and substituted alkylene. In some embodiments, R₆ is selected from the group comprising phenyl, substituted phenyl, pyridinyl, furanyl, substituted furanyl, and —NHC(═O)CH₃.

In some embodiments, L₁ is thiophenylene and the compound has a structure of Formula (II):

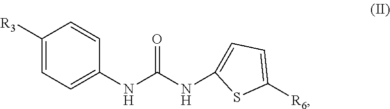

or a pharmaceutically acceptable salt or solvate thereof. In some embodiments, R₆ is selected from phenyl, substituted phenyl, or pyridinyl.

In some embodiments, R₃ is Cl, R₆ is phenyl or substituted phenyl, and wherein the compound of Formula (II) has a structure of Formula (IIa):

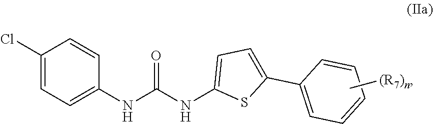

wherein: n is 0, 1, 2, 3, 4, or 5; and each R₇ is independently selected from the group comprising halo, nitro, hydroxy, cyano, alkyl, aryl, acyl, ester, alkoxy, sulfonyl, and dialkylamino; or a pharmaceutically acceptable salt or solvate thereof. In some embodiments, n is 1 or 2, and wherein each R₇ is halo, optionally chloro or fluoro. In some embodiments, n is 1 and R₇ is methoxy or methyl.

In some embodiments, the compound is selected from the group comprising:

1-(4-Chlorophenyl)-3-(5-phenylthiophen-2-Aurea (11),

1-(4-Chlorophenyl)-3-[5-(4-fluorophenyl)thiophen-2-yl]urea (18),

1-(4-Chlorophenyl)-3-[5-(3-fluorophenyl)thiophen-2-yl]urea (19),

1-(4-Chlorophenyl)-3-[5-(2,4-difluorophenyl)thiophen-2-yl]urea (20),

1-(4-Chlorophenyl)-3-[5-(2-chlorophenyl)thiophen-2-yl]urea (21),

1-(4-Chlorophenyl)-3-[5-(3-chlorophenyl)thiophen-2-yl]urea (22),

1-(4-Chlorophenyl)-3-[5-(4-chlorophenyl)thiophen-2-yl]urea (23),

1-(4-Chlorophenyl)-3-[5-(3,4-dichlorophenyl)thiophen-2-yl]urea (24),

1-(4-Chlorophenyl)-3-[5-(3,5-dichlorophenyl)thiophen-2-yl]urea (25),

3[5-(3-Acetylphenyl)thiophen-2-yl]-1-(4-chlorophenyl)urea (26),

Methyl 3-(5-{[(4-chlorophenyl)carbamoyl]amino}thiophen-2-yl)benzoate (27),

1-(4-Chlorophenyl)-3-[5-(3-methanesulfonylphenyl)thiophen-2-yl]urea (28),

1-(4-Chlorophenyl)-3-[5-(2-methoxyphenyl)thiophen-2-yl]urea (29),

1-(4-Chlorophenyl)-3-[5-(3-methoxyphenyl)thiophen-2-yl]urea (30),

1-(4-Chlorophenyl)-3-[5-(4-methoxyphenyl)thiophen-2-yl]urea (31),

1-(4-Chlorophenyl)-3-[5-(3-methylphenyl)thiophen-2-yl]urea (32),

1-(4-Chlorophenyl)-3-5-[3-(dimethylamino)phenyl]thiophen-2-yl)urea (33),

1-(4-Chlorophenyl)-3-[5-(pyridin-3-yl)thiophen-2-yl]urea (34), and

1-(4-Chlorophenyl)-3-[5-(pyridin-4-yl)thiophen-2-yl]urea (35);

or a pharmaceutically acceptable salt or solvate thereof.

In some embodiments, L₁ is ethylene or substituted ethylene and the compound of Formula (Ia) has a structure of Formula (III):

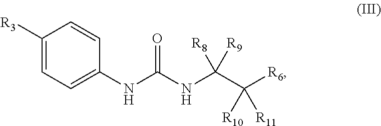

wherein: each of R₈, R₉, R₁₀, and R₁₁ are independently selected from the group comprising H, halo, and alkyl, or wherein two of R₈, R₉, R₁₀, and R₁₁ together from an alkylene group; or a pharmaceutically acceptable salt or solvate thereof. In some embodiments, R₃ is chloro, each of R₈, R₉, R₁₀, and

R₁₁ are H, R₆ is phenyl or substituted phenyl, and the compound of Formula (III) has a structure of Formula (IIIa):

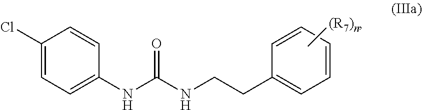

wherein: n is 0, 1, 2, 3, 4, or 5; and each R₇ is independently selected from the group comprising halo, nitro, hydroxyl, cyano, alkyl, perfluoroalkyl, aryl, acyl, ester, alkoxyl, sulfonyl, and dialkylamino; or a pharmaceutically acceptable salt or solvate thereof.

In some embodiments, each R₇ is independently selected from the group comprising fluoro, chloro, methyl, tert-butyl, phenyl, nitro, methoxy, dimethylamino, cyano, and trifluoromethyl. In some embodiments, the compound is selected from the group comprising:

trans-1-(4-Chlorophenyl)-3-(2-phenylcyclopropyl)urea (15),

cis-1-(4-Chlorophenyl)-3-(2-phenylcyclopropyl)urea (16),

3-(4-Chlorophenyl)-1-(2-phenylethyl)urea (44),

1-[2-(4-tert-Butylphenyl)ethyl]-3-(4-chlorophenyl)urea (45),

3-(4-Chlorophenyl)-1-[2-(4-phenylphenyl)ethyl]urea (46),

3-(4-Chlorophenyl)-1-[2-(4-chlorophenyl)ethyl]urea (47),

3-(4-Chlorophenyl)-1-[2-(4-nitrophenyl)ethyl]urea (48),

3-(4-Chlorophenyl)-1-[2-(4-hydroxy-3-methoxyphenyl)ethyl]urea (49),

3-(4-Chlorophenyl)-1-{2-[3-(dimethylamino)phenyl]ethyl}urea (50),

3-(4-Chlorophenyl)-1-{2-[4-(dimethylamino)phenyl]ethyl}urea (51),

3-(4-Chlorophenyl)-1-[2-(4-methanesulfonylphenyl)ethyl]urea (52),

3-(4-Chlorophenyl)-1-[2-(2-methoxyphenyl)ethyl]urea (53),

3-(4-Chlorophenyl)-1-[2-(3-methoxyphenyl)ethyl]urea (54),

3-(4-Chlorophenyl)-1-[2-(3-methoxyphenyl)ethyl]urea (55),

3-(4-Chlorophenyl)-1-[2-(3,4-dimethoxyphenyl)ethyl]urea (56),

3-(4-Chlorophenyl)-1-[2-(3,5-dimethoxyphenyl)ethyl]urea (57),

3-(4-Chlorophenyl)1-[2-(4-hydroxyphenyl)ethyl]urea (58),

3-(4-Chlorophenyl)1-[2-(4-methylphenyl)ethyl]urea (59),

3-(4-Chlorophenyl)1-[2-(3-methylphenyl)ethyl]urea (60),

3-(4-Chlorophenyl)1-[2-(2-fluorophenyl)ethyl]urea (61),

3-(4-Chlorophenyl)1-[2-(3-fluorophenyl)ethyl]urea (62),

3-(4-Chlorophenyl)1-[2-(4-fluorophenyl)ethyl]urea (63),

3-(4-Chlorophenyl)1-[2-(3,4-difluorophenyl)ethyl]urea (64),

3-(4-Chlorophenyl)1-[2-(2,4,6-trifluorophenyl)ethyl]urea (65),

3-(4-Chlorophenyl)1-[2-(2,3,4,5,6-pentafluorophenyl)ethyl]urea (66),

3-(4-Chlorophenyl)1-[2-(2-chlorophenyl)ethyl]urea (67),

3-(4-Chlorophenyl)1-[2-(3-chlorophenyl)ethyl]urea (68),

3-(4-Chlorophenyl)1-[2-(2,4-dichlorophenyl)ethyl]urea (69),

3-(4-Chlorophenyl)1-[2-(2-chloro-6-fluorophenyl)ethyl]urea (70),

3-(4-Chlorophenyl)1-[2-(4-bromophenyl)ethyl]urea (71),

3-(4-Chlorophenyl)1-[2-(4-cyanophenyl)ethyl]urea (72),

3-(4-Chlorophenyl)-1-{2-[2-(trifluoromethyl)phenyl]ethyl}urea (73),

3-(4-Chlorophenyl)-1-{2-[3-(trifluoromethyl)phenyl]ethyl}urea (74),

3-(4-Chlorophenyl)-1-{2-[4-(trifluoromethyl)phenyl]ethyl}urea (75),

3-(4-Chlorophenyl)1-[2-(pyridin-4-yl)ethyl]urea (76),

3-(4-Chlorophenyl)1-[2-(pyridin-3-yl)ethyl]urea (77)

3-(4-Chlorophenyl)1-[2-(pyridin-2-yl)ethyl]urea (78),

1-(4-Chlorophenyl)-3-[2-(5-methylfuran-2-yl)ethyl]urea (79),

3-(4-Chlorophenyl)-1-[2-(4-methylpiperazin-1-yl)ethyl]urea (80),

3-(4-Chlorophenyl)-1-[2-(piperidin-1-yl)ethyl]urea (81),

3-(4-Chlorophenyl)1-[2-(morpholin-4-yl)ethyl]urea (82),

1-(4-Chlorophenyl)-3-[2-(pyrrolidin-1-yl)ethyl]urea (83),

N-(2-{[(4-Chlorophenyl)carbamoyl]amino}ethyl)acetamide (84),

3-(4-Chlorophenyl)-1-(2-methyl-2-phenylpropyl)urea (38),

3-(4-Chlorophenyl)-1-(2,2-difluoro-2-phenylethyl)urea (39),

3-(4-Chlorophenyl)-1-(2-methyl1-phenylpropan-2-yl)urea (40),

1-(4-Chlorophenyl)-3-[(1-phenylcyclopropyl)methyl]urea (41), and

3-(1-Benzylcyclopropyl)-1-(4-chlorophenyl)urea (42);

or a pharmaceutically acceptable salt or solvate thereof.

In some embodiments, the compound is selected from the group comprising:

3-(4-Chlorophenyl)-1-{2-methoxy-5-[6-(pyrrolidin-1-yl)pyridin-2-yl]phenyl}urea (6),

1-(4-Chlorophenyl)-3-(4-phenylpyridin-2-yl)urea (7),

1-(4-Chlorophenyl)-3-(6-phenylpyridin-2-yl)urea (8),

1-(4-Chlorophenyl)-3-(5-phenylpyridin-3-yl)urea (9),

1-(4-Chlorophenyl)-3-(2-phenylpyridin-4-yl)urea (10),

1-(4-Chlorophenyl)-3-(4-phenylthiophen-2-yl)urea (12),

1-(4-Chlorophenyl)-3-(5-phenylthiophen-3-yl)urea (13),

1-(4-Chlorophenyl)-3-(5-phenyl-1,3-thiazol-2-yl)urea (14),

3-(4-Chlorophenyl)-1-[(3R)-1-phenylpiperidin-3-yl]urea (17),

1-Benzyl-3-(4-chlorophenyl)urea (36),

3-(4-Chlorophenyl)-1-(3-phenylpropyl)urea (37), and

trans-1-(4-Chlorophenyl)-3-[(2-phenylcyclopropyl)methyl]urea (43);

or a pharmaceutically acceptable salt or solvate thereof.

In some embodiments, the presently disclosed subject matter provides a pharmaceutical composition comprising one of the presently disclosed compounds and a pharmaceutically acceptable carrier.

In some embodiments, the presently disclosed subject matter provides a method of treating a cannabinoid 1 receptor (CB1R)-mediated disease or condition in a subject in need of treatment thereof, the method comprising administering to said subject a therapeutically effective amount of a compound of a compound of the presently disclosed subject matter or a pharmaceutical composition thereof. In some embodiments, the subject is a mammal, optionally a human.

In some embodiments, the disease or condition is selected from the group comprising drug addiction, obesity, cancer, pain, female infertility, memory loss, congnitive dysfunction, Parkinson's disease, dyskinesia, tardive dyskinesia, Alzheimer's disease, amyotrophic lateral sclerosis (ALS), Tourette's Syndrome, stroke, atherosclerosis, hypotension, intestinal hypoactivity in paralytic ileus, inflammation, osteoporosis, hypercholesterolemia, hyslipidemia, diabetes, retinopathy, glaucoma, anxiety, depression and other mood disorders, gastrointestinal disorders, and metabolic disorders. In some embodiments, the disease is obesity or drug addiction, optionally wherein the drug addiction is selected from cocaine addiction, opiod addiction, amphetamine addiction, cannabinoid addition, tobacco addiction, and alcohol addiction.

In some embodiments, the compound is a compound of Formula (II) or Formula (III). In some embodiments, the compound is selected from the group comprising:

1-(4-Chlorophenyl)-3-(5-phenylthiophen-2-yl)urea (11),

1-(4-Chlorophenyl)-3-[5-(2,4-difluorophenyl)thiophen-2-yl]urea (20),

1-(4-Chlorophenyl)-3-[5-(2-chlorophenyl)thiophen-2-yl]urea (21),

1-(4-Chlorophenyl)-3-[5-(4-methoxyphenyl)thiophen-2-yl]urea (31),

3-(4-Chlorophenyl)1-[2-(3-chlorophenyl)ethyl]urea (68), and

3-(4-Chlorophenyl)-1-{2-[3-(trifluoromethyl)phenyl]ethyl}urea (74);

or a pharmaceutically acceptable salt or solvate thereof.

In some embodiments, the presently disclosed subject matter provides a method of treating obesity in a subject in need of treatment thereof, the method comprising administering to said subject a therapeutically effective amount of a compound of the presently disclosed subject matter or a pharmaceutical composition thereof. In some embodiments, the compound is selected from the group comprising:

1-(4-Chlorophenyl)-3-(5-phenylthiophen-2-yl)urea (11),

1-(4-Chlorophenyl)-3-[5-(2,4-difluorophenyl)thiophen-2-yl]urea (20),

1-(4-Chlorophenyl)-3-[5-(2-chlorophenyl)thiophen-2-yl]urea (21),

1-(4-Chlorophenyl)-3-[5-(4-methoxyphenyl)thiophen-2-yl]urea (31),

3-(4-Chlorophenyl)1-[2-(3-chlorophenyl)ethyl]urea (68), and

3-(4-Chlorophenyl)-1-{2-[3-(trifluoromethyl)phenyl]ethyl}urea (74);

or a pharmaceutically acceptable salt or solvate thereof.

In some embodiments, the presently disclosed subject matter provides a method for preventing or inhibiting substance abuse and/or addiction, an addictive behavior, or of a symptom, behavior, or condition associated with substance abuse and/or addiction, the method comprising administering to a subject in need thereof a therapeutically effective amount of a compound of the presently disclosed subject matter or a pharmaceutical composition thereof. In some embodiments, the substance abuse and/or addiction is selected from cocaine addiction, opiod addiction, amphetamine addiction, cannabinoid addition, tobacco addiction, and alcohol addiction. In some embodiments, the administration prevents or inhibits relapse. In some embodiments, the compound is selected from the group comprising:

1-(4-Chlorophenyl)-3-(5-phenylthiophen-2-yl)urea (11),

1-(4-Chlorophenyl)-3-[5-(2,4-difluorophenyl)thiophen-2-yl]urea (20),

1-(4-Chlorophenyl)-3-[5-(2-chlorophenyl)thiophen-2-yl]urea (21),

1-(4-Chlorophenyl)-3-[5-(4-methoxyphenyl)thiophen-2-yl]urea (31),

3-(4-Chlorophenyl)1-[2-(3-chlorophenyl)ethyl]urea (68), and

3-(4-Chlorophenyl)-1-{2-[3-(trifluoromethyl)phenyl]ethyl}urea (74);

or a pharmaceutically acceptable salt or solvate thereof.

In some embodiments, the presently disclosed subject matter provides a method of modulating the activity of cannabinoid 1 receptor (CB1R), wherein the method comprises contacting a sample comprising CB1R with a compound of the presently disclosed subject matter or a pharmaceutical composition thereof.

It is an object of the presently disclosed subject matter to provide compounds of Formula (I) e.g., that have activity as CB1 allosteric modulators (e.g., CB1 negative allosteric modulators), as well as pharmaceutical compositions comprising the compounds, and methods of treating diseases, such as drug addiction, pain, obesity, inflammation, anxiety and depression, using the compounds or their pharmaceutical compositions. Certain objects of the presently disclosed subject matter having been stated hereinabove, which are addressed in whole or in part by the presently disclosed subject matter, other objects and aspects will become evident as the description proceeds when taken in connection with the accompanying Examples as best described herein below.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the below drawings.

FIG. 1A is a graph showing the activity of compound 11, an exemplary allosteric modulator of cannabinoid 1 receptor (CB1R), against 100 nanomolar (nM) CP55,940, a CB1R agonist, in a calcium mobilization assay in stable human CB1R-CHO-RD-HGA16 cells expressing human CB1R.

FIG. 1B is a graph showing the activity of compound 11, an exemplary allosteric modulator of cannabinoid 1 receptor (CB1R), against 100 nanomolar (nM) CP55,940, a CB1R agonist, in a sulfur-35 guanosine 5′-0-[gamma-thio]triphosphate [³⁵S]GTPyS binding assay in stable HEK293 cells stably expressing the human CB1R.

FIG. 10 is a graph showing the activity of compound 11, an exemplary allosteric modulator of cannabinoid 1 receptor (CB1R), against 100 nanomolar (nM) CP55,940, a CB1R agonist, in a sulfur-35 guanosine 5′-0-[gamma-thio]triphosphate [³⁵S]GTPyS binding assay in cerebella of male ICR mice.

FIG. 2 is a graph showing the intrinsic activities of cannabinoid 1 receptor (CB1R) allosteric modulators and a CB1-selective antagonist/inverse agonist (SR₁₄₁₇₁₆) in the absence of the CB1R agonist CP55,940. Activity is reported as the percentage (%) of basal sulfur-35 guanosine 5′-O-[gamma-thio]triphosphate ([³⁵S]GTPyS) binding as a function of the log of the modulator or agonist concentration (in moles per liter (M)). The allosteric modulators include PSNCBAM-1 (downward-pointing triangles) and four of the urea-based compounds of the presently disclosed subject matter, i.e., compound 14 (flowers), compound 9 (stars), compound 35 (diamonds), and compound 11 (upward-pointing triangles). Data for SR₁₄₁₇₁₆ is shown in circles.

FIG. 3A is a pair of graphs showing the behavior effects of compound 11 and compound 68 in a drug-induced reinstatement of cocaine-seeking study in rats. The effect of pretreatment with 10 milligrams per kilograms (mg/kg) of compound 68 (grey bars) or compound 11 (black bars) prior to cocaine-induced reinstatement of cocaine-seeking behavior on active lever responses is shown in the graph on the left, while the effects on inactive lever responses are shown in the graph on the right. In both graphs, the effect of treatment with vehicle (unfilled bars) is shown as a control. *p<0.05.

FIG. 3B is a graph showing the effects of compound 68 and compound 11 on locomotion in rats. Locomotion is presented as total distance (in millimeters (mm)) versus time (in minutes) after administration. The effects of treatment with vehicle is also shown as a control.

FIG. 4 is a graph of compound 68′s brain and plasma pharmacokinetic profiles following a single i.p. dose at 10 mg/kg to male Sprague-Dawley rats.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist, unless as otherwise specifically indicated.

### I. Definitions

Following long-standing patent law convention, the terms “a”, “an”, and “the” refer to “one or more” when used in this application, including the claims. Thus, for example, reference to “a solvent” includes mixtures of one or more solvents, two or more solvents, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the presently disclosed subject matter.

The term “about”, as used herein when referring to a measurable value such as an amount of weight, molar equivalents, time, temperature, etc. is meant to encompass in one example variations of ±20% or ±10%, in another example ±5%, in another example ±1%, and in yet another example ±0.1% from the specified amount, as such variations are appropriate to perform the disclosed methods.

The term “and/or” when used to describe two or more activities, conditions, or outcomes refers to situations wherein both of the listed conditions are included or wherein only one of the two listed conditions are included.

The term “comprising”, which is synonymous with “including,” “containing,” or “characterized by” is inclusive or open-ended and does not exclude additional, unrecited elements or method steps. “Comprising” is a term of art used in claim language, which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein the term “alkyl” refers to C1-020 inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C1-C8 alkyl), e.g., 1, 2, 3, 4, 5, 6, 7, or 8 carbon atoms. In some embodiments, “lower alkyl” can refer to C1-6 or C1-C5 alkyl groups. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C1-CC straight-chain or branched-chain alkyls. Alkyl groups can optionally be substituted (a “substituted alkyl”) with one or more alkyl group substituents, which can be the same or different. The term “alkyl group substituent” includes but is not limited to alkyl, substituted alkyl, halo, nitro, cyano, amino, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, cyano, amino, alkylamino, dialkylamino, ester, acyl, amide, sulfonyl, sulfate, and mercapto.

The term “alkenyl” refers to an alkyl group as defined above including at least one carbon-carbon double bond. Exemplary alkenyl groups include, but are not limited to, ethenyl, propenyl, butenyl, pentenyl, hexenyl, octenyl, butadienyl, and allenyl groups. Alkenyl groups can optionally be substituted with one or more alkyl group substitutents, which can be the same or different, including, but not limited to alkyl (saturated or unsaturated), substituted alkyl (e.g., halo-substituted and perhalo-substituted alkyl, such as but not limited to, —CF₃), cycloalkyl, halo, nitro, hydroxyl, carbonyl, carboxyl, acyl, alkoxyl, aryloxyl, aralkoxyl, thioalkyl, thioaryl, thioaralkyl, amino (e.g., aminoalkyl, aminodialkyl, aminoaryl, etc.), sulfonyl, and sulfinyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. In some embodiments, the cycloalkyl ring system comprises between 3 and 6 carbon atoms. The cycloalkyl group can be optionally partially unsaturated. The cycloalkyl group also can be optionally substituted with an alkyl group substituent as defined herein. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include, but are not limited to, cyclopropyl, cyclobutyl, cyclopentyl, cyclohexyl, cycloheptyl, and the like. Further, the cycloalkyl group can be optionally substituted with a linking group, such as an alkylene group as defined hereinbelow, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

Thus, as used herein, the term “substituted cycloalkyl” includes cycloalkyl groups, as defined herein, in which one or more atoms or functional groups of the cycloalkyl group are replaced with another atom or functional group, including for example, alkyl, substituted alkyl, halogen, aryl, substituted aryl, alkoxyl, hydroxyl, nitro, cyano, amino, alkylamino, dialkylamino, ester, acyl, amide, sulfonyl, sulfate, and mercapto.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds (i.e., “heteroaryl”). The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyridine, imidazole, benzimidazole, isothiazole, isoxazole, pyrazole, pyrazine, triazine, thiazole, pyrimidine, quinoline, isoquinoline, indole, carbazole, napthyl, and the like.

“Heterocyclic”, “heterocycle”, or “heterocyclo” as used herein alone or as part of another group, refers to an aliphatic (e.g., fully or partially saturated heterocyclo) or aromatic (e.g., heteroaryl) monocyclic- or a bicyclic-ring system comprising one or more heteroatoms (e.g., 1, 2, or 3 heteroatoms selected from oxygen, sulfur, and substituted or unsubstituted nitroten) inserted along the cyclic alkyl or aryl carbon chain. Monocyclic ring systems are exemplified by any 5- or 6-membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, ethylene oxide, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydropyran, tetrahydrothiophene (also known as thiolane), tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein.

Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, carbazole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like.

These rings include quaternized derivatives thereof and can be optionally substituted with one or more alkyl and/or aryl group substituents.

“Substituted heterocyclic” as used herein refers to a heterocyclic group wherein one or more hydrogen atom is replaced by an alkyl or aryl group substitutent.

The term “N-heterocycle” refers to a heterocycle wherein at least one of the heteroatoms is a nitrogen atom. Examples of N-heterocycles include, but are not limited to, azetidine, pyrrolidine, pyrrole, pyrroline, pyrazole, pyrazoline, pyrazolidine, piperidine, pyridine, piperazine, pyrazine, pyrimidine, pyridazine, morpholine, imidazole, benzimidazole, imidazoline, imidazolidine, indole, carbazole, quinoline, isoquinoline, oxazole, thiazole, isothiazole, and thiazine.

“Substituted N-heterocycle” refers to a N-heterocycle wherein one or more hydrogen is replaced by an alkyl or aryl group substituent. The term “heteroaryl” ref eres to an aromatic monocyclic- or a bicyclic-ring system (a fused, bridged or spirocyclic ring system) comprising one or more heteroatoms (e.g., 1, 2, or 3 heteroatoms selected from oxygen, sulfur, and substituted or unsubstituted nitrogen, wherein N-oxides, sulfur oxides and dioxides are permissible heteroatom substitutions) inserted along the cyclic aryl carbon chain. In some embodiments, the monocyclic heteroaryl group is a five to seven membered aromatic ring. Representative heteroaryl groups include, but are not limited to, furan, thiophene, pyrrole, imidazole, pyrazole, triazole, tetrazole, oxazole, isoxazole, oxadiazole, thiaciazole, isothiazole, pyridine, pyridazine, pyrazine, pyrimidine, quinoline, isoquinoline, benzofuran, benzoxazole, benzothiophene, indole, indazole, benzimidazole, imidazopyridine, pyrazolopyrindine, and pyrazolopyrimidine.

The term “substituted heteroaryl” refers to a heteroaryl group as defined herein wherein one or more hydrogen atoms is replaced by an aryl group substituent. “Aralkyl” refers to an aryl-alkyl- or an -alkyl-aryl group wherein aryl and alkyl are as previously described and can include substituted aryl and substituted alkyl. Thus, “substituted aralkyl” can refer to an aralkyl group comprising one or more alkyl or aryl group substituents. Exemplary aralkyl groups include benzyl, phenylethyl, and naphthylmethyl.

“Alkylene” can refer to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated (i.e., include alkene or alkyne groups) and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ₗ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂-O—); and ethylenedioxyl (—O—(CH₂)₂—O—). An alkylene group can have about 2 to about 3 carbon atoms and can further have 6-20 carbons. “Arylene” refers to a bivalent aryl group, which can be substituted or unsubstituted.

The term “aralkylene” refers to a bivalent group that comprises a combination of alkylene and arylene groups (e.g., -arylene-alkylene-, alkylene-arylene-alkylene-, arylene-alkylene-arylene-, etc.). Similarly, the terms “cycloalkylene”, “heterocycloalkylene” and “heteroarylene” refer to bivalent cycloalkyl, heterocyclic, and heteroaryl groups, which can optionally be substituted with one or more alkyl or aryl group substitutents.

As used herein, the term “acyl” refers to an organic carboxylic acid group wherein the -OH of the carboxylic acid group has been replaced with another substituent. Thus, an acyl group can be represented by RC(═O)—, wherein R is an alkyl, substituted alkyl, aralkyl, substituted aralkyl, aryl or substituted aryl group as defined herein. As such, the term “acyl” specifically includes arylacyl groups, such as a phenacyl group. Specific examples of acyl groups include acetyl (i.e., —C(═O)CH₃) and benzoyl.

“Alkoxyl” refers to an alkyl-O— group wherein alkyl is as previously described, including substituted alkyl. The term “alkoxyl” as used herein can refer to, for example, methoxyl, ethoxyl, propoxyl, isopropoxyl, butoxyl, t-butoxyl, and pentoxyl. The terms “oxyalkyl” and “alkoxy” can be used interchangably with “alkoxyl”.

“Aryloxyl” and “aryloxy” refer to an aryl-O— group wherein the aryl group is as previously described, including a substituted aryl. The term “aryloxyl” as used herein can refer to phenyloxyl or hexyloxyl, and to alkyl, substituted alkyl, or alkoxyl substituted phenyloxyl or hexyloxyl.

“Aralkyloxyl” or “aralkoxy” refer to an aralkyl-O— group wherein the aralkyl group is as previously described. An exemplary aralkyloxyl group is benzyloxyl.

The term “carbonyl” refers to the group —C(═O)—. The term “carbonyl carbon” refers to a carbon atom of a carbonyl group. Other groups such as, but not limited to, acyl groups, anhydrides, aldehydes, esters, lactones, amides, ketones, carbonates, and carboxylic acids, include a carbonyl group.

The terms “carboxyl” and “carboxylic acid” refer to the —C(═O)OH or —C(═O)O— group.

The term “acid chloride” can refer to the —C(═O)Cl group.

The terms “halo” or “halogen” as used herein refer to fluoro, chloro, bromo, and iodo groups.

The term “haloalkyl” refers to an alkyl group as defined herein substituted by one or more halo groups.

The term “perhaloalkyl” refers to an alkyl group as defined herein wherein all C-H bonds are replaced by carbon-halogen bonds. The term “perfluoroalkyl” refers to an alkyl group wherein all C—H bonds are replaced by C—F bonds. An exemplary perfluoroalkyl group is trifluoromethyl (—CF₃). The term “sulfonyl” refers to the —S(═O)₂R group, wherein R is alkyl, substituted alkyl, aralkyl, substituted aralkyl, aryl, or substituted aryl. The term “alkylsulfonyl” refers to the —S(═O)₂R group, wherein R is alkyl or substituted alkyl. In some embodiments, the sulfonyl group is —S(═O)₂CH₃.

The term “ester” refers to the R′—O—C(═O)— group, wherein the carbonyl carbon is attached to another carbon atom and wherein R′ is alkyl, cycloalkyl, aralkyl, or aryl, wherein the alkyl, cycloalkyl, aralkyl, or aryl are optionally substituted. The term “esterifying” can refer to forming an ester by contacting a compound containing a carboxylic acid or derivative thereof (e.g., an acid chloride) and a compound containing a hydroxyl group (e.g., an alcohol or a phenol).

The term “amide” refers to a compound comprising the structure R′—NR″—C(═O)—R, wherein R is alkyl, substituted alkyl, aralkyl, substituted aralkyl, aryl or substituted aryl, and wherein R′ and R″ are independently hydrogen, alkyl, aralkyl, or aryl, wherein the alkyl, aralkyl, or aryl are optionally substituted. In some embodiemnts, R′ is alkyl, substituted alkyl, aralkyl, substituted aralkyl, aryl, or substituted aryl.

The term “urea” as used herein refers to a compound comprising the structure R-NR'—C(═O)—NR′—R, wherein each R is independently alkyl, substituted alkyl, aralkyl, substituted aralkyl, aryl, or substituted aryl, and wherein each R′ is independently H, alkyl, substituted alkyl, aralkyl, substituted aralkyl, aryl, or substituted aryl.

A structure represented generally by a formula such as:

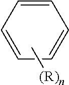

as used herein refers to a ring structure, for example, but not limited to a 3-carbon, a 4-carbon, a 5-carbon, a 6-carbon, and the like, aliphatic and/or aromatic cyclic compound comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure:

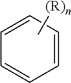

wherein n is an integer from 0 to 2 comprises compound groups including, but not limited to:

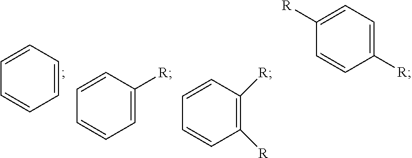

and the like.

When a named atom of an aromatic ring or a heterocyclic aromatic ring is defined as being “absent,” the named atom is replaced by a direct bond. When the linking group or spacer group is defined as being absent, the linking group or spacer group is replaced by a direct bond.

A line crossed by a wavy line, e.g., in the structure:

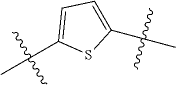 [SMILES: *c1ccc(*)s1]

indicates the site where a chemical moiety can bond to another group.

The term “amine” refers to a molecule having the formula N(R)₃, or a protonated form thereof, wherein each R is independently H, alkyl, substituted alkyl, aryl, substituted aryl, aralkyl, substituted aralkyl, or wherein two R groups together form an alkylene or arylene group. The term “primary amine” refers to an amine wherein at least two R groups are H. The term “secondary amine” refers to an amine wherein only one R group is H. The term “alkylamine” can refer to an amine wherein two R groups are H and the other R group is alkyl or substituted alkyl. “Dialkylamine” can refer to an amine where two R groups are alkyl. “Arylamine” can refer to an amine wherein one R group is aryl. Amines can also be protonated, i.e., have the formula [NH(R)₃]⁺.

The term “amino” refers to the group —N(R)2 wherein each R is independently H, alkyl, substituted alkyl, aryl, substituted aryl, aralkyl, or substituted aralkyl. The terms “aminoalkyl” and “alkylamino” can refer to the group —N(R)₂ wherein each R is H, alkyl or substituted alkyl, and wherein at least one R is alkyl or substituted alkyl. The term “dialkylamino” refers to an aminoalkyl group where both R groups are alkyl or substituted alkyl, which can be the same or different.

The terms “acylamino” and “aminoacyl” refer to the —N(R)—C(═O)R′ group, wherein R is selected from H, alkyl, substituted alkyl, aralkyl, substituted aralkyl, aryl, and substituted aryl, and wherein R′ is selected from alkyl, substituted alkyl, aralkyl, substituted aralkyl, aryl, and substituted aryl.

The term “cyano” refers to the —C≡N group.

The terms “hydroxyl” and “hydroxy” refer to the —OH group.

The terms “mercapto” and “thiol” refer to the —SH group.

The term “oxo” refers to a compound described previously herein wherein a carbon atom is replaced by an oxygen atom.

The term “nitro” refers to the —NO2 group.

The term “thioalkyl” can refer to the group —SR, wherein R is selected from H, alkyl, substituted alkyl, aralkyl, substituted aralkyl, aryl, and substituted aryl. Similarly, the terms “thioaralkyl” and “thioaryl” refer to —SR groups wherein R is aralkyl and aryl, respectively.

When the term “independently selected” is used, the substituents being referred to (e.g., R groups, such as groups R₁ and R₂, or groups X and Y), can be identical or different. For example, both R₁ and R₂ can be substituted alkyls, or R₁ can be hydrogen and R₂ can be a substituted alkyl, and the like.

Anamed “R”, “R′,” “X,” “Y,” “Y′”, “A,” “A′”, “B,” “L,” or “Z” group will generally have the structure that is recognized in the art as corresponding to a group having that name, unless specified otherwise herein. For the purposes of illustration, certain representative “R,” “X,” and “Y” groups as set forth above are defined below. These definitions are intended to supplement and illustrate, not preclude, the definitions that would be apparent to one of ordinary skill in the art upon review of the present disclosure.

The terms “treatment” and “treating” and the like as used herein refers to any treatment of a disease and/or condition in an animal or mammal, particularly a human, and includes: (i) preventing a disease, disorder and/or condition from occurring in a person which can be predisposed to the disease, disorder and/or condition, or at risk for being exposed to an agent that can cause the disease, disorder, and/or condition; but, has not yet been diagnosed as having it; (ii) inhibiting the disease, disorder and/or condition, i.e., arresting its development; and (iii) relieving the disease, disorder and/or condition, i.e., causing regression of the disease, disorder and/or condition.

“Protecting group” as used herein includes any suitable protecting group; “protected form” refers to a substituent in which an atom such as hydrogen has been removed and replaced with a corresponding protecting group. Protecting groups are known. See generally T. H. Greene and P. G. M. Wuts, Protective Groups in Organic Synthesis, 3rd edition, John Wiley & Sons, New York (1999). Examples include but are not limited to: hydroxy protecting groups (for producing the protected form of hydroxy); carboxy protecting groups (for producing the protected form of carboxylic acid); amino-protecting groups (for producing the protected form of amino); sulfhydryl protecting groups (for producing the protected form of sulfhydryl); etc. Particular examples include but are not limited to: benzyloxycarbonyl, 4-nitrobenzyloxycarbonyl, 4-bromobenzyloxycarbonyl, 4-m ethoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-f u rfu ryloxycarbonyl, al lyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-triehloroethoxymethyl, 2-(trimethylsilyl)ethoxymethyl, methanesulfonyl, para-toluenesulfonyl, trimethylsilyl, triethylsilyl, triisopropylsilyl, acetyl (Ac), benzoyl (Bn), and trimethylsilyl (TMS), and the like; formyl, acetyl, benzoyl, pivaloyl, t-butylacetyl, phenylsulfonyl, benzyl, t-butyloxycarbonyl (Boc), and benzyloxycarbonyl (Cbz) and the like; and hemithioacetals such as 1-ethoxyethyl and methoxymethyl, thioesters, or thiocarbonates and the like.

The term “allosteric modulator” as used herein refers to a compound (or “ligand”) that binds to a site on a macromolecule (e.g., a receptor) that is distinct from the orthosteric site (i.e., the primary binding site of the macromolecule). Allosteric modulators can indirectly influence the effects of an orthosteric or primary ligand that binds at the orthosteric site. For example, an allosteric modulator of the CB1 receptor can bind to the receptor at the site distinct to the orthosteric sites leading to a change in receptor conformation.

As a result, interactive properties of the receptor with respect to orthosteric ligand(s) and cellular host environment can be modified in either a positive or negative direction, respectively referred to as positive allosteric modulators (“PAMs”) and negative allosteric modulators (“NAMs”). Allosteric modulators can exhibit the following pharmacological properties: (i) affinity modulation, where the resulting conformation can alter either association or dissociation rate of on orthosleric ligand; (ii) efficacy modulation, where the allosteric effect can modify intracellular response and lead to a change in the signaling capacity of the orthosteric ligand; and/or (iii) agonism/inverse agonism, where the allosteric modulator can perturb receptor signaling in either a positive or negative direction, irrespective of presence of orthosteric modulator.

### H. General Considerations

Preclinical and clinical studies suggest that the blockade of CB1R is a promising strategy for the treatment of many common drugs of abuse, as well as a number of other conditions, including for example, but not limited to, obesity, anxiety, cancer, inflammation, Parkinson's disease, osteoporosis, female infertility, metabolic disorders, pain, stroke, hypotension, and intestinal hypoactivity. Unfortunately, to date, psychiatric side effects such as depression, anxiety, or even suidical ideation, have restricted the use of CB1R antagonist/inverse agonists in the clinic.

Despite this setback, CB1R continues to be a target for drug development and various strategies have been explored to overcome the psychiatric adverse effects of CB1R signaling while preserving beneficial therapeutic effects. Like many GPCRs, CB1R displays a high level of constitutive activity in the absence of exogenous ligands in both neurons (see Pan et al., Mol. Pharmacol. 1998, 54, 1064-1072; and Hillard et al., FEBS Lett. 1999, 459, 277-281) and non-neuronal cells. See Bouaboula et al., J. Biol. Chem. 1997, 272, 22330-22339. As the constitutive activity is important to maintain cellular homeostatsis, the adverse effects of the CB1R antagonist/inverse agonist rimonabant are thought to be derived from its CB1R inverse agonism that reduces CB1R basal tone. Therefore, it has been postulated that neutral antagonists which attenuate CB1R signaling in overactive conditions but leave the CB1R basal level unchanged could have fewer side effects. See Greasley and Clapham, Eur. J. Pharmacol. 2006, 553, 1-9. Peripherally resitricted antagonists which do not cross the blood-brain barrier have also shown promising therapeutic efficacy in the treatment of obesity and diabetes without the liability of the central nervous system (CNS) side effects. See Chorvat, Bioorg. Med. Chem. Lett. 2013, 23, 4751-4760.

In addition, the discovery of CB1R allosteric binding sites has offered a promising alternative approach to modulate CB1R signaling for therapeutic benefits. Allosteric modulators target CB1R at the allosteric binding site, offering several advantages to orthosteric ligands, such as better receptor subtype selectivity, lower risk of overdosing to the the “ceiling” effect, and more transient pharmacological effects as a result of their dependence on the presence of endocannabinoids. See Nguyen et al., Med. Res. Rev. 2017, 37, 441-474.

The structures of two previously studed CB1R negative allosteric modulators, Org27569 (1) and PSNCBAM-1 (2), are shown in Scheme 1, below. See German et al., J. Med. Chem. 2014, 57, 7758-7769; and Nguyen et al., Bioorg. Med. Chem. 2015, 23, 2195-2203. Compound 2, for example, exhibits positive binding cooperativity with CP55,940, a cannabinoid receptor agonist that mimics the effects of THC; reduces the efficacy of agonists in several functional assays; and reduces food intake and body weight in rats. See Horswill et al., Br. J. Pharmacol. 2007, 152, 805-814.

Structure-activity relationship (SAR) efforts on compound 2 have indicated that the pyrrolidinyl ring is not required for CB1R modulatory activity and that the pyridinyl ring can be replaced with substituted phenyl rings or five-membered heterocycles, such as in RTICBM-229 (5), also shown in Scheme 1, which exhibits greater potency than compound 2 in the [³⁵S]GTPγS binding assay and a higher maximum binding level in the [³H]CP55,940 binding assay.

See German et al., J. Med. Chem. 2014, 57, 7758-7769; Nguyen et al., J. Med. Chem. 2017, 60, 7410-7424; and Nguyen et al., ACS Chem. Neurosci. 2019, 10, 518-527.

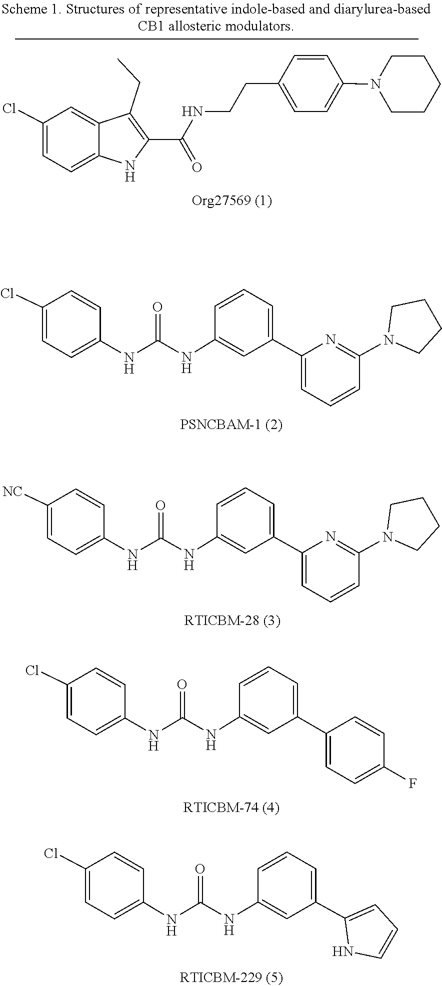 [SMILES: CCc1c(C(=O)NCCc2ccc(N3CCCCC3)cc2)[nH]c2ccc(Cl)cc12 ; O=C(Nc1ccc(Cl)cc1)Nc1cccc(-c2cccc(N3CCCC3)n2)c1 ; N#Cc1ccc(NC(=O)Nc2cccc(-c3cccc(N4CCCC4)n3)c2)cc1 ; O=C(Nc1ccc(Cl)cc1)Nc1cccc(-c2ccc(F)cc2)c1 ; O=C(Nc1ccc(Cl)cc1)Nc1cccc(-c2ccc[nH]2)c1]

Efforts to optimize diaryl urea-based compound 2 also led to compound RTICBM-74 (4). See Scheme 1. Compound 4 attenuates prime-induced restatement of cocaine seeking and while RTICBM-28 (3), in which the chloro group in the outer phenyl ring of 2 is replaced by cyano, reduces THC's potency in drug discrimination, demonstrating the therapeutic potential of these CB1R allosteric modulators for the treatment of the relapse of cocaine addiction (see Nguyen et al., J. Med. Chem. 2017, 60, 7410-7424) and THC dependence. See Gamage et al., Neuropharmacology 2017, 125, 365-375.

Overall, SAR of the outer phenyl ring indicated that the 4-position favors electron withdrawing functionalities. See German et al., J. Med. Chem. 2014, 57, 7758-7769.

The presently disclosed subject matter is based in part on further efforts to extend the SAR understanding of the diaryl urea-based scaffold of 2 by structural optimization at the middle phenyl ring. See Scheme 2, below. The compounds described herein are believed to be the first series where substitution/replacement of the middle phenyl ring of compound 2 has been studied. More particularly, the presently disclosed compounds are those where the middle phenyl ring is replaced with a variety of heteroaryl rings, including pyridine, thiophene and thiazole, as well as non-aromatic rings, such as cyclopropyl or piperidinyl rings, and non-cyclic aliphatic groups (e.g., ethylene).

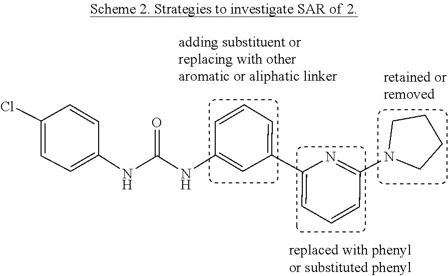 [SMILES: O=C(Nc1ccc(Cl)cc1)Nc1cccc(-c2cccc(N3CCCC3)n2)c1]

As described in the Example 2 below, replacement of the middle phenyl ring of compound 2 with heteroaryl or alkylene moieties improved or retained the CB1R modulation activities. Some of the presently disclosed compounds, e.g., 1-(4-chlorophenyl)-3-(5-phenylthiophen-2-yl)urea (11) and 1-(4-chlorophenyl)-3-[5-(2,4-difluorophenyl)thiophen-2-yl]urea (20), had better in vitro potencies at the CB1 receptor than compound 2, while maintaining good selectivity over the CB2 receptor in the calcium mobilization and [³⁵S]GTP-γ-S binding assays. As described in Example 3, below, two exemplary compounds of the presently disclosed subject matter, i.e., compounds 11 and 20, exhibited better metabolic stability in liver enzymes than compound 2, while compound 11 was more soluble than compound 2. As described in Example 4, exemplary compound 68 demonstrated good in vivo efficacy at 10 mg/kg when administered via intraperitoneal injection in a reinstatement of cocaine-seeking behavior model in rats. Further, the presently disclosed compounds, unlike CB1 receptor inverse agonist/antagonists, such as SR141716, advantageously display little to no inverse agonism. Accordingly, they are expected to be less likely to cause psychiatric side effects, which is a significant advancement over existing compounds.

### III. Urea-Based CB1R Allosteric Modulators

In some embodiments, the presently disclosed subject matter provides a compound having a structure of Formula (I):

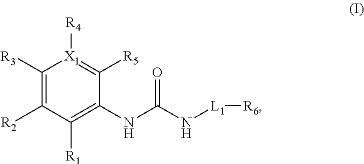

wherein:

X₁ is —C— or —N—;

each of R₁, R₂, R₃, and R₅ is independently selected from the group comprising H, alkyl (e.g., C1-C6 alkyl), substituted alkyl (e.g., C1-C6 substituted alkyl), halo, haloalkyl (e.g., C1-C6 haloalkyl, such as C1-C6 perfluoroalkyl), alkoxy (e.g., C1-C6 alkoxy), nitro, and cyano, or wherein R₂ and R₃ together form an alkylene group (e.g., an oxo-containing alkylene group, such as —OCH₂O—, or an alkene-containing alkylene group, such as —CH═CH—CH═CH—);

R₄ is present (i.e., when X₁ is —C—) or absent (i.e., when X₁ is —N—), and when present is selected from the group comprising H, alkyl (e.g., C1-C6 alkyl), substituted alkyl (e.g., C1-C6 substituted alkyl), halo, haloalkyl (e.g., C1-C6 haloalkyl, such as C1-C6 perfluoroalkyl), alkoxy (e.g., C1-C6 alkoxy), nitro, and cyano;

L₁ is selected from the group comprising alkylene (e.g., C1-C6 saturated alkylene), substituted alkylene (e.g., C1-C6 substituted alkylene), cycloalkylene (e.g., cyclopropylene), substituted cycloalkylene (e.g., substituted cyclopropylene), heterocycloalkylene (e.g., piperidinylene), substituted arylene (e.g., substituted phenylene), heteroarylene (e.g., thiophenylene, pyridinylene, thioazolylene), and substituted heteroarylene; and

R₆ is selected from the group comprising aryl (e.g., phenyl), substituted aryl (e.g., substituted phenyl), heteroaryl (e.g., pyridinyl or furanyl), substituted heteroaryl, alkylamino, dialkylamino (e.g., dimethylamino), acylamino (i.e., —NHC(═O)CH₃), N-heterocycle (e.g., piperazinyl, piperidinyl, morpholinyl, or pyrrolidinyl) and substituted N-heterocycle; or a pharmaceutically acceptable salt or solvate thereof.

In some embodiments, X₁ is —C—. In some embodiments, R₁ and R₅ are each H.

In some embodiments, each of R₁, R₂, R₄, and R₅ is H and the compound of Formula (I) has a structure of Formula (Ia):

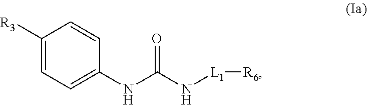

wherein R₃, L1 and R₆ are as defined for the compounds of Formula (I); or a pharmaceutically acceptable salt or solvate thereof.

In some embodiments, R₃ is an electron withdrawing group. The term “electron-withdrawing” refers to an atom, substituent, or moiety that draws electron density from neighboring atoms toward itself (e.g., via inductive or resonance effects) as compared to a hydrogen atom. In some embodiments, R₃ is an electron withdrawing group such as, but not limited to, halo (e.g., fluoro, chloro or bromo), trihalomethyl (e.g., trifluoromethyl), formyl, acyl (e.g., acetyl), —C(═O)OH, ester (e.g., methyl ester (—C(═O)—OCH₃), cyano, and nitro. In some embodiments, R₃ is halo, nitro, or cyano. In some embodiments, R₃ is Cl.

In some embodiments, L₁ is selected from the group comprising thiophenylene, pyridinylene, thiazolylene, alkylene (e.g., methylene, ethylene, propylene, or butylene), and substituted alkylene (e.g., alkyl-substituted alkylene or halo-substituted alkylene). For example, L₁ can be heteroarylene selected from:

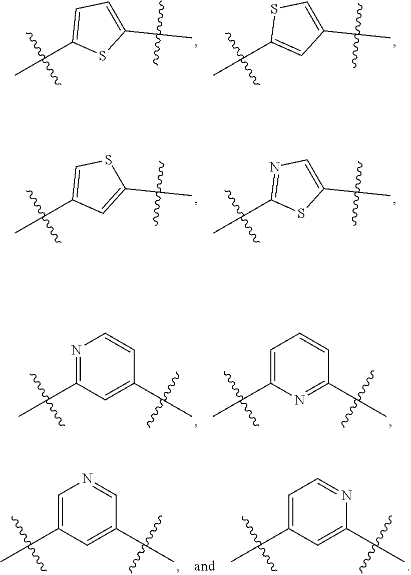

In some embodiments, R₆ is selected from the group comprising aryl (e.g., phenyl or napthyl), substituted aryl (e.g., substituted phenyl), heteroaryl (e.g., pyridinyl or furanyl), substituted heteroaryl (e.g., substituted furanyl), and acylamino. In some embodiments, R₆ is selected from substituted aryl, heteroaryl, substituted heteroaryl, and acylamino. In some embodiments, R₆ is selected from substituted phenyl, pyridinyl, furanyl, and —NHC(═O)CH₃.

In some embodiments, L₁ is thiophenylene and the compound has a structure of Formula (II):

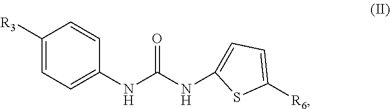

wherein R₃ and R₆ are as defined above for the compounds of Formula (I); or a pharmaceutically acceptable salt or solvate thereof.

In some embodiments, R₃ is an electron withdrawing group. In some embodiments, R₃ is halo. In some embodiments, R₃ is chloro.

In some embodiments, R₆ is phenyl, substituted phenyl or pyridinyl. In some embodiments, R₆ is substituted phenyl or pyridinyl (e.g., 3-pyridinyl or 4-pyridinyl). For example, R₆ can be phenyl substituted by one or more halo, alkyl (e.g., C1-C6 alkyl), alkoxy (e.g., C1-C6 alkoxy), acyl, ester, sulfonyl, or dialkylamino groups. In some embodiments, R₆ is phenyl substituted by one or more fluoro, chloro, bromo, methyl, ethyl, methoxy, ethoxy, acyl, —C(═O)OMe, —S(═O)2Me, or dimethylamino groups. In some embodiments, R₆ is mono- or di-substituted phenyl.

In some embodiments, R₃ is Cl, R₆ is phenyl or substituted phenyl, and the compound of Formula (II) has a structure of Formula (IIa):

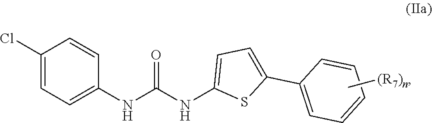

wherein:

n is 0, 1, 2, 3, 4, or 5; and

each R₇ is independently selected from the group comprising halo, nitro, hydroxyl, cyano, alkyl, aryl, acyl, ester, alkoxyl, sulfonyl, and dialkylamino; or a pharmaceutically acceptable salt or solvate thereof. In some embodiments, n is 1, 2, 3, 4, or 5.

In some embodiments, n is 1 or 2. In some embodiments, R₇ is halo.

In some embodiments, R₇ is chloro or fluoro.

In some embodiments, n is 1 and R₇ is chloro, fluoro, methoxy, dimethylamino, or methyl. In some embodiments, R₇ is chloro, fluoro, methoxy, or methyl.

In some embodiments, the compound of Formula (II) is selected from the group comprising:
1-(4-Chlorophenyl)-3-(5-phenylthiophen-2-yl)urea (11),
1-(4-Chlorophenyl)-3-[5-(4-fluorophenyl)thiophen-2-yl]urea (18),
1-(4-Chlorophenyl)-3-[5-(3-fluorophenyl)thiophen-2-yl]urea (19),
1-(4-Chlorophenyl)-3-[5-(2,4-difluorophenyl)thiophen-2-yl]urea (20),
1-(4-Chlorophenyl)-3-[5-(2-chlorophenyl)thiophen-2-yl]urea (21),
1-(4-Chlorophenyl)-3-[5-(3-chlorophenyl)thiophen-2-yl]urea (22),
1-(4-Chlorophenyl)-3-[5-(4-chlorophenyl)thiophen-2-yl]urea (23),
1-(4-Chlorophenyl)-3-[5-(3,4-dichlorophenyl)thiophen-2-yl]urea (24),
1-(4-Chlorophenyl)-3-[5-(3,5-dichlorophenyl)thiophen-2-yl]urea (25),
3-[5-(3-Acetylphenyl)thiophen-2-yl]-1-(4-chlorophenyl)urea (26),
Methyl 3-(5-{[(4-chlorophenyl)carbamoyl]amino}thiophen-2-yl)benzoate (27),
1-(4-Chlorophenyl)-3-[5-(3-methanesulfonylphenyl)thiophen-2-yl]urea (28),
1-(4-Chlorophenyl)-3-[5-(2-methoxyphenyl)thiophen-2-yl]urea (29),
1-(4-Chlorophenyl)-3-[5-(3-methoxyphenyl)thiophen-2-yl]urea (30),
1-(4-Chlorophenyl)-3-[5-(4-methoxyphenyl)thiophen-2-yl]urea (31),
1-(4-Chlorophenyl)-3-[5-(3-methylphenyl)thiophen-2-yl]urea (32),
1-(4-Chlorophenyl)-3-{5-[3-(dimethylamino)phenyl]thiophen-2-yl}urea (33),
1-(4-Chlorophenyl)-3-[5-(pyridin-3-yl)thiophen-2-yl]urea (34), and
1-(4-Chlorophenyl)-3-[5-(pyridin-4-yl)thiophen-2-yl]urea (35);
or a pharmaceutically acceptable salt or solvate thereof.

In some embodiments, the compound of Formula (II) is other than 1-(4-chlorophenyl)-3-(5-phenylthiophen-2-yl)urea (11). Thus, in some embodiments, the compound of Formula (II) is selected from the group comprising:
1-(4-Chlorophenyl)-3-[5-(4-fluorophenyl)thiophen-2-yl]urea (18),
1-(4-Chlorophenyl)-3-[5-(3-fluorophenyl)thiophen-2-yl]urea (19),
1-(4-Chlorophenyl)-3-[5-(2,4-difluorophenyl)thiophen-2-yl]urea (20),
1-(4-Chlorophenyl)-3-[5-(2-chlorophenyl)thiophen-2-yl]urea (21),
1-(4-Chlorophenyl)-3-[5-(3-chlorophenyl)thiophen-2-yl]urea (22),
1-(4-Chlorophenyl)-3-[5-(4-chlorophenyl)thiophen-2-yl]urea (23),
1-(4-Chlorophenyl)-3-[5-(3,4-dichlorophenyl)thiophen-2-yl]urea (24),
1-(4-Chlorophenyl)-3-[5-(3,5-dichlorophenyl)thiophen-2-yl]urea (25),
3-[5-(3-Acetylphenyl)thiophen-2-yl]-1-(4-chlorophenyl)urea (26),
Methyl 3-(5-{[(4-chlorophenyl)carbamoyl]amino}thiophen-2-yl)benzoate (27),
1-(4-Chlorophenyl)-3-[5-(3-methanesulfonylphenyl)thiophen-2-yl]urea (28),
1-(4-Chlorophenyl)-3-[5-(2-methoxyphenyl)thiophen-2-yl]urea (29),
1-(4-Chlorophenyl)-3-[5-(3-methoxyphenyl)thiophen-2-yl]urea (30),
1-(4-Chlorophenyl)-3-[5-(4-methoxyphenyl)thiophen-2-yl]urea (31),
1-(4-Chlorophenyl)-3-[5-(3-methylphenyl)thiophen-2-yl]urea (32),
1-(4-Chlorophenyl)-3-{5-[3-(dimethylamino)phenyl]thiophen-2-yl}urea (33),
1-(4-Chlorophenyl)-3-[5-(pyridin-3-yl)thiophen-2-yl]urea (34), and
1-(4-Chlorophenyl)-3-[5-(pyridin-4-yl)thiophen-2-yl]urea (35);
or a pharmaceutically acceptable salt or solvate thereof.

In some embodiments, the compound of Formula (II) is selected from the group comprising 1-(4-Chlorophenyl)-3-(5-phenylthiophen-2-yl)urea (11), 1-(4-Chlorophenyl)-3-[5-(2,4-difluorophenyl)thiophen-2-yl]urea (20), 1-(4-Chloro-phenyl)-3-[5-(2-chlorophenyl)thiophen-2-yl]urea (21), and 1-(4-Chlorophenyl)-3-[5-(4-methoxyphenyl)thiophen-2-yl]urea (31). In some embodiments, the compound of Formula (II) is selected from 1-(4-Chlorophenyl)-3-[5-(2,4-difluorophenyl)thiophen-2-yl]urea (20), 1-(4-Chloro-phenyl)-3-[5-(2-chlorophenyl)thiophen-2-yl]urea (21), and 1-(4-Chlorophenyl)-3-[5-(4-methoxyphenyl)thiophen-2-yl]urea (31).

In some embodiments, L₁ in Formula (I) is ethylene or substituted ethylene and the compound of Formula (Ia) has a structure of Formula (III):

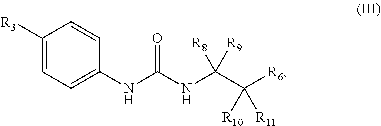

wherein R₃ and R₆ are as defined above for the compounds of Formula (I), and each of R₈, R₉, R₁₀, and R₁₁ are independently selected from the group comprising H, halo, and alkyl (e.g., C1-C6 alkyl), or wherein two of R₈, R₉, R₁₀, and R₁₁ together from an alkylene group; or a pharmaceutically acceptable salt or solvate thereof. In some embodiments, two of R₈, R₉, R₁₀, and R₁₁ together form a methylene or ethylene group. In some embodiments, R₁₀ and R₁₁ together comprise an ethylene group, thereby forming a cyclopropyl ring together with the carbon atom to which R₁₀ and R₁₁ are attached. In some embodiments, R₈ and R₁₀ together comprise a methylene group, thereby forming a cyclopropyl ring together with the carbon atoms to which R₈ and R₁₀ are attached. In some embodiments, each of R₈, R₉, R₁₀, and R₁₁ are independently selected from H, methyl, and fluoro. In some embodiments, two of R₈, R₉, R₁₀, and R₁₁ (e.g., R₈ and R₉ or R₁₀ and R₁₁) are methyl or fluoro. In some embodiments, each of R₈, R₉, R₁₀, and R₁₁ is H.

In some embodiments, R₃ is an electron withdrawing group. In some embodiments, R₃ is halo. In some embodiments, R₃ is chloro.

In some embodiments, R₆ is phenyl, substituted phenyl, heteroaryl (e.g., furanyl or pyridinyl), or substituted heteroaryl (e.g., methyl-substituted furanyl). In some embodiments, R₆ is phenyl, substituted phenyl, or methyl-substituted furanyl. In some embodiments, R₆ is phenyl or substituted phenyl. For example, R₆ can be phenyl substituted by one or more halo, alkyl (e.g., C1-C6 alkyl), alkoxy (e.g., C1-C6 alkoxy), acyl (e.g., acetyl), ester, sulfonyl, or dialkylamino groups. In some embodiments, R₆ can be phenyl substituted by one or more fluoro, chloro, bromo, methyl, ethyl, methoxy, ethoxy, acyl, —C(═O)OMe, —S(═O)₂Me, or dimethylamino groups. In some embodiments, R₆ is mono- or di-substituted phenyl. In some embodiments, R₆ is tri-, or penta-fluoro-substituted phenyl.

In some embodiments, R₃ is chloro, each of R₈, R₉, R₁₀, and R₁₁ are H, R₆ is phenyl or substituted phenyl, and the compound of Formula (III) has a structure of Formula (IIIa):

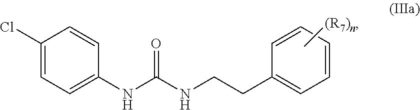

wherein:

n is 0, 1, 2, 3, 4, or 5; and

each R₇ is independently selected from the group comprising halo, nitro, hydroxyl, cyano, alkyl (e.g., C1-C6 alkyl), perfluoroalkyl (e.g., C1-C6 perfluoroalkyl), aryl, acyl, ester, alkoxy (e.g., C1-C6 alkoxy), sulfonyl, and dialkylamino; or a pharmaceutically acceptable salt or solvate thereof. In some embodiments, each R₇ is independently selected from the group comprising fluoro, chloro, methyl, tert-butyl, phenyl, nitro, methoxy, dimethylamino, cyano, and trifluoromethyl. In some embodiments, n is 1, 2, 3, 4, or 5.

In some embodiments, the compound of Formula (III) is selected from the group comprising:
trans-1-(4-Chlorophenyl)-3-(2-phenylcyclopropyl)urea (15),
cis-1-(4-Chlorophenyl)-3-(2-phenylcyclopropyl)urea (16),
3-(4-Chlorophenyl)1-(2-phenylethyl)urea (44),
1-[2-(4-tert-Butylphenyl)ethyl]-3-(4-chlorophenyl)urea (45),
3-(4-Chlorophenyl)-1-[2-(4-phenylphenyl)ethyl]urea (46),
3-(4-Chlorophenyl)1-[2-(4-chlorophenyl)ethyl]urea (47),
3-(4-Chlorophenyl)1-[2-(4-nitrophenyl)ethyl]urea (48),
3-(4-Chlorophenyl)1-[2-(4-hydroxy-3-methoxyphenyl)ethyl]urea (49),
3-(4-Chlorophenyl)-1-{2-[3-(dimethylamino)phenyl]ethyl}urea (50),
3-(4-Chlorophenyl)-1-{2-[4-(dimethylamino)phenyl]ethyl}urea (51),
3-(4-Chlorophenyl)1-[2-(4-methanesulfonylphenyl)ethyl]urea (52),
3-(4-Chlorophenyl)1-[2-(2-methoxyphenyl)ethyl]urea (53),
3-(4-Chlorophenyl)1-[2-(3-methoxyphenyl)ethyl]urea (54),
3-(4-Chlorophenyl)1-[2-(3-methoxyphenyl)ethyl]urea (55),
3-(4-Chlorophenyl)1-[2-(3,4-dimethoxyphenyl)ethyl]urea (56),
3-(4-Chlorophenyl)1-[2-(3,5-dimethoxyphenyl)ethyl]urea (57),
3-(4-Chlorophenyl)1-[2-(4-hydroxyphenyl)ethyl]urea (58),
3-(4-Chlorophenyl)1-[2-(4-methylphenyl)ethyl]urea (59),
3-(4-Chlorophenyl)1-[2-(3-methylphenyl)ethyl]urea (60),
3-(4-Chlorophenyl)1-[2-(2-fluorophenyl)ethyl]urea (61),
3-(4-Chlorophenyl)1-[2-(3-fluorophenyl)ethyl]urea (62),
3-(4-Chlorophenyl)1-[2-(4-fluorophenyl)ethyl]urea (63),
3-(4-Chlorophenyl)1-[2-(3,4-difluorophenyl)ethyl]urea (64),
3-(4-Chlorophenyl)1-[2-(2,4,6-trifluorophenyl)ethyl]urea (65),
3-(4-Chlorophenyl)1-[2-(2,3,4,5,6-pentafluorophenyl)ethyl]urea (66),
3-(4-Chlorophenyl)1-[2-(2-chlorophenyl)ethyl]urea (67),
3-(4-Chlorophenyl)1-[2-(3-chlorophenyl)ethyl]urea (68),
3-(4-Chlorophenyl)1-[2-(2,4-dichlorophenyl)ethyl]urea (69),
3-(4-Chlorophenyl)1-[2-(2-chloro-6-fluorophenyl)ethyl]urea (70),
3-(4-Chlorophenyl)1-[2-(4-bromophenyl)ethyl]urea (71),
3-(4-Chlorophenyl)1-[2-(4-cyanophenyl)ethyl]urea (72),
3-(4-Chlorophenyl)-1-{2-[2-(trifluoromethyl)phenyl]ethyl}urea (73),
3-(4-Chlorophenyl)-1-{2-[3-(trifluoromethyl)phenyl]ethyl}urea (74),
3-(4-Chlorophenyl)-1-{2-[4-(trifluoromethyl)phenyl]ethyl}urea (75),
3-(4-Chlorophenyl)1-[2-(pyridin-4-yl)ethyl]urea (76),
3-(4-Chlorophenyl)1-[2-(pyridin-3-yl)ethyl]urea (77)
3-(4-Chlorophenyl)1-[2-(pyridin-2-yl)ethyl]urea (78),
1-(4-Chlorophenyl)-3-[2-(5-methylfuran-2-yl)ethyl]urea (79),
3-(4-Chlorophenyl)-1-[2-(4-methylpiperazin-1-yl)ethyl]urea (80),
3-(4-Chlorophenyl)-1-[2-(piperidin-1-yl)ethyl]urea (81),
3-(4-Chlorophenyl)1-[2-(morpholin-4-yl)ethyl]urea (82),
1-(4-Chlorophenyl)-3-[2-(pyrrolidin-1-yl)ethyl]urea (83),
N-(2-{[(4-Chlorophenyl)carbamoyl]amino}ethyl)acetamide (84),
3-(4-Chlorophenyl)-1-(2-methyl-2-phenylpropyl)urea (38),
3-(4-Chlorophenyl)-1-(2,2-difluoro-2-phenylethyl)urea (39),
3-(4-Chlorophenyl)-1-(2-methyl1-phenylpropan-2-yl)urea (40),
1-(4-Chlorophenyl)-3-[(1-phenylcyclopropyl)methyl]urea (41), and
3-(1-Benzylcyclopropyl)-1-(4-chlorophenyl)urea (42); or
a pharmaceutically acceptable salt or solvate thereof.

In addition to the compounds of Formula (II) and (III) above (which are also compounds of Formula (I)), in some embodiments, the compound of Formula (I) is selected from the group comprising: 3-(4-Chlorophenyl)-1-{2-methoxy-5-[6-(pyrrolidin1-yl)pyridin-2-yl]phenyl}urea (6),
1-(4-Chlorophenyl)-3-(4-phenylpyridin-2-yl)urea (7),
1-(4-Chlorophenyl)-3-(6-phenylpyridin-2-yl)urea (8),
1-(4-Chlorophenyl)-3-(5-phenylpyridin-3-yl)urea (9),
1-(4-Chlorophenyl)-3-(2-phenylpyridin-4-yl)urea (10),
1-(4-Chlorophenyl)-3-(4-phenylthiophen-2-yl)urea (12),
1-(4-Chlorophenyl)-3-(5-phenylthiophen-3-yl)urea (13),
1-(4-Chlorophenyl)-3-(5-phenyl-1,3-thiazol-2-yl)urea (14),
3-(4-Chlorophenyl)1-[(3R)1-phenylpiperidin-3-yl]urea (17),
1-Benzyl-3-(4-chlorophenyl)urea (36),
3-(4-Chlorophenyl)1-(3-phenylpropyl)urea (37), and trans-1-(4-Chlorophenyl)-3-[(2-phenylcyclopropyl)methyl]urea (43); or a pharmaceutically acceptable salt or solvate thereof.

In some embodiments, the compound of Formula (I) is other than compound 9, 11, or 14. In some embodiments, the compound of Formula (I) is selected from the group including compounds 6-8, 10, 12, 13, 17, 36, 37, and 43.

In some embodiments, the compound of Formula (I), (Ia), (II), (11a), (III) or (IIIa) has an IC₅₀ for human CB1R (hCB1R) of 1,000 nM or less (e.g., of 1,000 nM or less, of 500 nM or less, of 400 nM or less, of 300 nM or less, of 250 nM or less, of 200 nM or less, or of 150 nM or less) as measured using a calcium mobilization assay. In some embodiments, the compound of Formula (I), (Ia), (II), (IIa), (III), or (IIIa) has an IC₅₀ for hCB1R of 100 nM or less as measured using a calcium mobilization assay. In some embodiments, the compound is selected from:
1-(4-Chlorophenyl)-3-(5-phenylthiophen-2-yl)urea (11),
1-(4-Chlorophenyl)-3-(4-phenylthiophen-2-yl)urea (12),
1-(4-Chlorophenyl)-3-(5-phenylthiophen-3-yl)urea (13),
1-(4-Chlorophenyl)-3-(5-phenyl-1,3-thiazol-2-yl)urea (14),
1-(4-Chlorophenyl)-3-[5-(4-fluorophenyl)thiophen-2-yl]urea (18),
1-(4-Chlorophenyl)-3-[5-(3-fluorophenyl)thiophen-2-yl]urea (19),
1-(4-Chlorophenyl)-3-[5-(2,4-difluorophenyl)thiophen-2-yl]urea (20),
1-(4-Chlorophenyl)-3-[5-(2-chlorophenyl)thiophen-2-yl]urea (21),
1-(4-Chlorophenyl)-3-[5-(3-chlorophenyl)thiophen-2-yl]urea (22),
1-(4-Chlorophenyl)-3-[5-(4-chlorophenyl)thiophen-2-yl]urea (23),
1-(4-Chlorophenyl)-3-[5-(3,5-dichlorophenyl)thiophen-2-yl]urea (25),
1-(4-Chlorophenyl)-3-[5-(2-methoxyphenyl)thiophen-2-yl]urea (29),
1-(4-Chlorophenyl)-3-[5-(3-methoxyphenyl)thiophen-2-yl]urea (30),
1-(4-Chlorophenyl)-3-[5-(4-methoxyphenyl)thiophen-2-yl]urea (31),
1-(4-Chlorophenyl)-3-[5-(3-methylphenyl)thiophen-2-yl]urea (32),
1-(4-Chlorophenyl)-3-{5-[3-(dimethylamino)phenyl]thiophen-2-yl}urea (33),
1-(4-Chlorophenyl)-3-[5-(pyridin-3-yl)thiophen-2-yl]urea (34),
1-(4-Chlorophenyl)-3[5-(pyridin-4-yl)thiophen-2-yl]urea (35),
3-(4-Chlorophenyl)-1-(2-phenylethyl)urea (44),
1-[2-(4-tert-Butylphenyl)ethyl]-3-(4-chlorophenyl)urea (45),
3-(4-Chlorophenyl)-1[-2-(4-nitrophenyl)ethyl]urea (48),
3-(4-Chlorophenyl)-1[-2-(3-methylphenyl)ethyl]urea (60),
3-(4-Chlorophenyl)-1-[2-(3-fluorophenyl)ethyl]urea (62),
3-(4-Chlorophenyl)-1-[2-(3,4-difluorophenyl)ethyl]urea (64),
3-(4-Chlorophenyl)-1-[2-(2,4,6-trifluorophenyl)ethyl]urea (65),
3-(4-Chlorophenyl)1-[2-(2,3,4,5,6-pentafluorophenyl)ethyl]urea (66),
3-(4-Chlorophenyl)1-[2-(3-chlorophenyl)ethyl]urea (68), and
3-(4-Chlorophenyl)-1-{2-[3-(trifluoromethyl)phenyl]ethyl}urea (74); or a pharmaceutically acceptable salt or solvate thereof.

In some embodiments, the compound is selected from:
1-(4-Chlorophenyl)-3-(5-phenylthiophen-2-yl)urea (11),
1-(4-Chlorophenyl)-3-[5-(2,4-difluorophenyl)thiophen-2-yl]urea (20),
1-(4-Chlorophenyl)-3-[5-(2-chlorophenyl)thiophen-2-yl]urea (21),
1-(4-Chlorophenyl)-3-[5-(4-methoxyphenyl)thiophen-2-yl]urea (31),
3-(4-Chlorophenyl)1-[2-(3-chlorophenyl)ethyl]urea (68), and
3-(4-Chlorophenyl)-1-{2-[3-(trifluoromethyl)phenyl]ethyl}urea (74); or a pharmaceutically acceptable salt or solvate thereof.

In some embodiments, the compound is other than compound 9, compound 11, or compound 14.

As indicated above, it is to be understood that the presently disclosed compounds can comprise pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N, N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g. , lysine and arginine dicyclohexylamine and the like.

Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like.

Furthermore, the presently disclosed compounds can have one or more polymorph or amorphous crystalline forms, which, as such, are intended to be included in the scope of the presently disclosed subject matter. In addition, some of the compounds of the presently disclosed subject matter can form solvates with water (i.e., hydrates) or common organic solvents (e.g., tetrahydrofuran (THF), ethanol (EtOH), methanol (MeOH), etc.). Accordingly, solvates of the compounds of Formula (I), (Ia), (II), (IIa), (III), and (IIIa) are also intended to be encompassed within the scope of the presently disclosed subject matter.

### IV. Pharmaceutical Compositions

The compounds disclosed herein can be formulated in accordance with the routine procedures adapted for a desired administration route. Accordingly, in some embodiments, the presently disclosed subject matter provides a pharmaceutical composition comprising a therapeutically effective amount of a compound as disclosed hereinabove (e.g., a compound of Formula (I), (Ia), (II), (IIa), (III) and/or Formula (IIIa)), or a pharmaceutically acceptable salt or solvate thereof, and a pharmaceutically acceptable carrier. The therapeutically effective amount can be determined by testing the compounds in an in vitro or in vivo model and then extrapolating therefrom for dosages in subjects of interest, e.g., humans. The therapeutically effective amount should be enough to exert a therapeutically useful effect in the absence of undesirable side effects in the subject to be treated with the composition.

Pharmaceutically acceptable carriers are well known to those skilled in the art and include, but are not limited to, from about 0.01 to about 0.1M and preferably 0.05M phosphate buffer or 0.8% saline. Such pharmaceutically acceptable carriers can be aqueous or non-aqueous solutions, suspensions and emulsions. Examples of non-aqueous solvents suitable for use in the presently disclosed subject matter include, but are not limited to, propylene glycol, polyethylene glycol, vegetable oils such as olive oil, and injectable organic esters such as ethyl oleate. Aqueous carriers suitable for use in the presently disclosed subject matter include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the presently disclosed subject matter can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Parenteral carriers suitable for use in the presently disclosed subject matter include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the presently disclosed subject matter can be mixed as needed with disintegrants, diluents, granulating agents, lubricants, binders and the like using conventional techniques known in the art. The carriers can also be sterilized using methods that do not deleteriously react with the compounds, as is generally known in the art. The compounds disclosed herein can take such forms as suspensions, solutions or emulsions in oily or aqueous vehicles, and can contain formulatory agents such as suspending, stabilizing and/or dispersing agents. The compounds disclosed herein can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt). Alternatively, the active ingredient can be in powder form for constitution with a suitable vehicle, e.g., sterile pyrogen-free water, before use. Suitable formulations for each of these methods of administration can be found, for example, in Remington: The Science and Practice of Pharmacy, A. Gennaro, ed., 20th edition, Lippincott, Williams & Wilkins, Philadelphia, Pa.

For example, formulations for parenteral administration can contain as common excipients sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers can be useful excipients to control the release of active compounds. Other potentially useful parenteral delivery systems include ethylene-vinyl acetate copolymer particles, osmotic pumps, implantable infusion systems, and liposomes. Formulations for inhalation administration contain as excipients, for example, lactose, or can be aqueous solutions containing, for example, polyoxyethylene-9-auryl ether, glycocholate and deoxycholate, or oily solutions for administration in the form of nasal drops, or as a gel to be applied intranasally. Formulations for parenteral administration can also include glycocholate for buccal administration, methoxysalicylate for rectal administration, or citric acid for vaginal administration.

Further, formulations for intravenous administration can comprise solutions in sterile isotonic aqueous buffer. Where necessary, the formulations can also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachet indicating the quantity of active agent. Where the compound is to be administered by infusion, it can be dispensed in a formulation with an infusion bottle containing sterile pharmaceutical grade water, saline or dextrose/water. Where the compound is administered by injection, an ampule of sterile water for injection or saline can be provided so that the ingredients can be mixed prior to administration.

Suitable formulations further include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compounds can further be formulated for topical administration. Suitable topical formulations include one or more compounds in the form of a liquid, lotion, cream or gel. Topical administration can be accomplished by application directly on the treatment area. For example, such application can be accomplished by rubbing the formulation (such as a lotion or gel) onto the skin of the treatment area, or by spray application of a liquid formulation onto the treatment area.

In some formulations, bioimplant materials can be coated with the compounds so as to improve interaction between cells and the implant.

Formulations of the compounds can contain minor amounts of wetting or emulsifying agents, or pH buffering agents. The formulations comprising the compound can be a liquid solution, suspension, emulsion, tablet, pill, capsule, sustained release formulation, or powder. The compounds can be formulated as a suppository, with traditional binders and carriers such as triglycerides.

Oral formulations can include standard carriers such as pharmaceutical grades of mannitol, lactose, starch, magnesium stearate, polyvinyl pyrollidone, sodium saccharine, cellulose, magnesium carbonate, etc.

In some embodiments, the pharmaceutical composition comprising the compound of the presently disclosed subject matter can include an agent which controls release of the compound, thereby providing a timed or sustained release compound.

### V. Methods of Treatment

As described hereinabove, CB1 and CB2 cannabinoid receptors belong to the G protein-coupled receptor (GCPR) family, a receptor super-family with a distinctive pattern of seven transmembrane domains, which inhibits N-type calcium channels and/or adenylate cyclase to inhibit Q-type calcium channels. CB1 receptors are present in the CNS, predominately expressed in brain regions associated with memory and movement such as the hippocampus (memory storage), cerebellum (coordination of motor function, posture and balance), basal ganglia (movement control), hypothalamus (thermal regulation, neuroendocrine release, appetite), spinal cord (nociception), cerebral cortex (emesis) and periphery regions such as lymphoid organs (cell mediated and innate immunity), vascular smooth muscle cells (blood pressure), gastrointestinal tract (innate antiinflammatory response in the gastrointestinal tract (e.g., in the esophagus, duodenum, jejunum, ileum and colon), controlling esophageal and gastrointestinal motility), lung smooth muscle cells (bronchodilation), eye ciliary body (intraocular pressure). CB2 receptors appear to be primarily expressed peripherally in lymphoid tissue (cell mediated and innate immunity), peripheral nerve terminals (peripheral nervous system), spleen immune cells (immune system modulation) and retina (intraocular pressure). CB2 mRNA is also found in the CNS in cerebellar granule cells (coordinating motor function),

Thus, cannabinoid receptor allosteric modulators, including the compounds of Formula (I), (Ia), (II), (IIa), (Ill), and (IIIa), are useful for treating, ameliorating or preventing a cannabinoid receptor mediated syndrome, disorder or disease including, but not limited to, controlling appetite, regulating metabolism, diabetes, glaucoma-associated intraocular pressure, pain, social and mood disorders, seizure-related disorders, substance abuse disorders, learning, cognition and/or memory disorders, bowel disorders, respiratory disorders, locomotor activity disorders, movement disorders, immune disorders or inflammation disorders, controlling organ contraction and muscle spasm, enhancing learning, cognition and/or memory, regulating cell growth (e.g., treating cancer), providing neuroprotection and the like.

Appetite related syndromes, disorders or diseases include obesity, overweight condition, anorexia, bulimia, cachexia, dysregulated appetite and the like. Obesity related syndromes, disorders or diseases include obesity as a result of genetics, diet, food intake volume, metabolic syndrome, disorder or disease, hypothalmic disorder or disease, age, reduced activity, abnormal adipose mass distribution, abnormal adipose compartment distribution and the like. Metabolism related syndromes, disorders or diseases include metabolic syndrome, dyslipidemia, elevated blood pressure, diabetes, insulin sensitivity or resistance, hyperinsulinemia, hypercholesterolemia, hyperlipidemias, hypertriglyceridemias, atherosclerosis, hepatomegaly, steatosis, abnormal alanine aminotransferase levels, inflammation, atherosclerosis and the like.

Diabetes related syndromes, disorders or diseases include glucose dysregulation, insulin resistance, glucose intolerance, hyperinsulinemia, dyslipidemia, hypertension, obesity and the like.

Type II diabetes mellitus (non-insulin-dependent diabetes mellitus (NIDDM)) is a metabolic disorder (i.e., a metabolism related syndrome, disorder or disease) in which glucose dysregulation and insulin resistance results in chronic, long-term medical complications for both adolescents and adults affecting the eyes, kidneys, nerves and blood vessels and can lead to blindness, end-stage renal disease, myocardial infarction or limb amputation and the like. Glucose dysregulation includes the inability to make sufficient insulin (abnormal insulin secretion) and the inability to effectively use insulin (resistance to insulin action in target organs and tissues). Individuals suffering from Type II diabetes mellitus have a relative insulin deficiency. That is, in such individuals, plasma insulin levels are normal to high in absolute terms, although they are lower than predicted for the level of plasma glucose that is present. Type II diabetes mellitus is characterized by the following clinical signs or symptoms: persistently elevated plasma glucose concentration or hyperglycemia; polyuria; polydipsia and/or polyphagia; chronic microvascular complications such as retinopathy, nephropathy and neuropathy; and macrovascular complications such as hyperlipidemia and hypertension. These micro-and macro-vascular complications can lead to blindness, end-stage renal disease, limb amputation and myocardial infarction. Insulin Resistance Syndrome (IRS) (also referred to as Syndrome X, Metabolic Syndrome or Metabolic Syndrome X) is a disorder that presents risk factors for the development of Type II diabetes and cardiovascular disease including glucose intolerance, hyperinsulinemia, insulin resistance, dyslipidemia (e.g. high triglycerides, low HDL-cholesterol and the like), hypertension and obesity.

Social or mood related syndromes, disorders or diseases include depression, anxiety, psychosis, social affective disorders or cognitive disorders and the like. Substance abuse related syndromes, disorders or diseases include drug abuse, drug withdrawal, alcohol abuse, alcohol withdrawal, nicotine withdrawal, cocaine abuse, cocaine withdrawal, heroin abuse, heroin withdrawal and the like. Learning, cognition or memory related syndromes, disorders or diseases include memory loss or impairment as a result of age, disease, side effects of medications (adverse events) and the like.

Muscle spasm syndromes, disorders or diseases include multiple sclerosis, cerebral palsy and the like. Locomotor activity and movement syndromes, disorders or diseases include stroke, Parkinson's disease, multiple sclerosis, epilepsy and the like. Bowel related syndromes, disorders or diseases include bowel dysmotility associated disorders (either accompanied by pain, diarrhea or constipation or without), irritable bowel syndrome (and other forms of bowel dysmotility and the like), inflammatory bowel diseases (such as ulcerative colitis, Crohn's disease and the like) and celiac disease. Respiratory related syndromes, disorders or diseases include chronic pulmonary obstructive disorder, emphysema, asthma, bronchitis and the like. Immune or inflammation related syndromes, disorders or diseases include allergy, rheumatoid arthritis, dermatitis, autoimmune disease, immunodeficiency, chronic neuropathic pain and the like.

Cell growth related syndromes, disorders or diseases include cancer, such as, but not limited to endometrial cancer, hepatocellular cancer, ovarian cancer, breast cancer, pancreatic cancer, colorectal cancer, lung cancer, prostate cancer, and renal cell carcinoma, and the like. Pain related syndromes, disorders or diseases include central and peripheral pathway mediated pain, bone and joint pain, migraine headache associated pain, cancer pain, menstrual cramps, labor pain and the like. Neurodegenerative related syndromes, disorders or diseases include Parkinson's Disease, multiple sclerosis, epilepsy, ischemia or secondary biochemical injury collateral to traumatic head or brain injury, brain inflammation, eye injury or stroke and the like.

Based on the antagonistic activity, the presently disclosed compounds can be useful as agents for prevention and/or treatment of a CB1 receptor-mediated diseases such as psychosis including schizophrenia, anxiety disorders, stress, depression, epilepsy, neurodegenerative disorders, spinocerebellar disorders, cognitive disorders, craniocerebral trauma, panic attack, peripheral neuropathy, glaucoma, migraine, Parkinson's disease,

Alzheimer's disease, Huntington's disease, Raynaud's syndrome, tremor, obsessive-compulsive disorders (OCD), amnesia, geriatric dementia, thymic disorders, Tourette's syndrome, tardive dyskinesia, bipolar disorders, cancer, drug-induced dyskinesia, dystonia, septic shock, hemorrhagic shock, hypotension, insomnia, immunological diseases including inflammations, multiple screlosis, emesis, diarrhea, asthma, appetite disorders such as bulimarexia, anorexia and the like, obesity, non insulin-dependent diabetes mellitus (NIDDM), memory disorders, urinary disorders, cardiovascular disorders, infertility disorders, infections, demyelination-related diseases, neuroinflammation, viral encephalitis, cerebral vascular incidents, cirrhosis of the liver or gastrointestinal disorders including intestinal transit disorders. In addition, the presently disclosed compounds can be used as agents for the treatment of substance addiction. For example, in some embodiments, the presently disclosed compounds can be used to treat withdrawal from a chronic treatment, alcohol dependence or drug abuse (e.g., an opioid, barbiturate, marijuana, cocaine, heroin, amphethamine, phencyclidine, a hallucinogenic agent, a benzodiazepine compound and the like). Furthermore, the presently disclosed compounds can be useful as an agent for enhancing analgesic activity of analgesic or narcotic drugs and the like; or an agent for smoking cessation (withdrawal from smoking or nicotine dependence).

Accordingly, in some embodiments, the presently disclosed subject matter provides a method of treating a CB1R mediated disease or condition in a subject in need thereof, wherein the method comprises administering to the subject a therapeutically effective amount of a compound of one or more of Formula (I), (Ia), (II), (IIa), (III), or (IIIa), or a pharmaceutically acceptable salt or solvate thereof or a pharmaceutical composition thereof.

With respect to the methods of the presently disclosed subject matter, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the presently disclosed subject matter indicate effectiveness with respect to all vertebrate species which are to be included in the term “subject.” In this context, a vertebrate is understood to be any vertebrate species in which treatment of a CB1R-mediated condition is desirable. As used herein, the term “subject” includes both human and animal subjects. Thus, veterinary therapeutic uses are provided in accordance with the presently disclosed subject matter.

As such, the presently disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided is the treatment of livestock, including, but not limited to, domesticated swine, ruminants, ungulates, horses (including race horses), poultry, and the like. In some embodiments, the subject is a human.

In some embodiments, the CB1R-mediated disease or condition is selected from the group including, but not limited to, drug addiction (e.g., alcohol, tobacco or other substance addiction), obesity, cancer (e.g., endometrial cancer, hepatocellular cancer, ovarian cancer, breast cancer, pancreatic cancer, colorectal cancer, lung cancer, prostate cancer, renal cell carcinoma, or desmotrophic small round cell tumors), pain (e.g., chronic pain, acute pain, somatic pain, visceral pain, meropathic pain, inflammatory pain), female infertility, memory loss, congnitive dysfunction, Parkinson's disease, dyskinesia, tardive dyskinesia, Alzheimer's disease, amyotrophic lateral sclerosis (ALS), Tourette's Syndrome, stroke, atherosclerosis, hypotension, intestinal hypoactivity in paralytic ileus, inflammation, osteoporosis, hypercholesterolemia, hyslipidemia, diabetes, retinopathy, glaucoma, anxiety, depression and other mood disorders, gastrointestinal disorders, and metabolic disorders.

The treatment of anxiety, for example, can include the treatment of anxiety disorders, such as, but not limited to, generalized anxiety disorder (GAD), post-traumatic stress disorder (PTSD), obsessive-compulsive disorder (OCD), panic disorder, social phobia, agoraphobia, or other more particular phobias. Eating disorders include, but are not limited to, anorexia, bulimia, and binge eating. Mood disorders include, but are not limited to, manic depression (bipolar disorder), major depression, and post-partum depression. Cognitive dysfunction includes disorders such as, for example, dementia, Attention Deficit Hyperactivity Disorder (ADHD), autism and Autism Spectrum Disorders (ASD), Down's Syndrome, traumatic brain injury (TBI), dyslexia, and the like. Alcoholism and substance abuse-related disorders can include abuse and/or addiction to alcohol, nicotine, or other drugs (e.g., opiates (e.g., heroin), cannabinoids, inhalants and psychostimulants such as cocaine, amphetamine and methamphetamine).

More particularly, diseases or conditions wherein inhibition of biological activity at, or signalling via, the CB1R is desirable include, but are not limited to obesity, alcoholism, and other substance abuse and/or addiction-related disorders. Thus, in some embodiments, the presently disclosed subject matter provides a method of treating obesity in a subject in need thereof, wherein the method comprises administering to the subject a compound of one of Formulas (I), (Ia), (II), (IIa), (III), or (IIIa) or a pharmaceutically acceaptable salt or solvate thereof or a pharmaceutical composition thereof.

In some embodiments, the subject is a human.

By way of further example, the presently disclosed CB1R allosteric modulators can find application in the treatment of substance use, abuse and/or addiction (including drug, alcohol and nicotine addiction), addictive behavior and symptoms and conditions associated with substance abuse and addiction, as exemplified herein. In some embodiments, the addiction is to at least one of nicotine, ethanol, cocaine, opiods, amphetamines, marijuana, or a synthetic cannabinoid agonist.

Addiction to substances such as alcohol, opiates, cannabinoids, nicotine marijuana, and psychostimulants is typically associated with a number of adverse or negative behaviors exhibited by addicts, which behaviors can serve to exacerbate, prolong or induce relapse into use or abuse of the substance, reinforce or exacerbate the addiction, or induce relapse into addiction and addictive behavior patterns. Other examples of negative behaviors associated with substance use or addiction include anxiety, dysphoria, stress reactivity, and cue reactivity. One particular problem with alcoholism, as with substance addiction in general, is the chronic relapsing nature of the disorder. This behavior pattern can be effectively modelled in rodents, where numerous studies have demonstrated the ability of drug priming, psychological stress or the re-presentation of cues previously associated with drug availability to reinstate drug-seeking behavior following extinction, even in the absence of subsequent drug rewards.

In some embodiments, the presently disclosed subject matter provides a method for the prevention or inhibition of substance abuse and/or addiction, an addictive behavior, or of a symptom, behavior, or condition associated with substance abuse and/or addiction, the method comprising administering to a subject in need thereof an effective amount of a CB1R allosteric modulator compound as disclosed herein (i.e., a compound of Formula (I), (Ia), (II), (IIa), (III), or (IIIa) or a pharmaceutically acceptable salt or solvate thereof) or a pharmaceutically acceptable composition comprising such a compound. In some embodiments, the subject is a human.

In some embodiments, the behavior associated with substance abuse and/or addiction comprises substance use (i.e., self-administration) and/or substance seeking behavior. In some embodiments, the substance abuse and/or addiction comprises alcohol abuse and/or addiction (i.e., alcoholism). In some embodiments, the substance abuse and/or addiction comprises nicotine abuse and/or addiction. In some embodiments, the substance abuse and/or addiction comprises opiate abuse and/or addiction. In some embodiments, the behavior associated with substance abuse or addiction is relapse.

In some embodiments, the compound administered in one of the methods of the presently disclosed subject matter is a compound of Formula (II), Formula (IIa), Formula (III), or Formula (IIIa). In some embodiments, the compound is selected from 7, 9, 10-15, 17-35, 44-48, 51, 54, 55, 59-75 and 79. In some embodiments, the compound is selected from 11-14, 18-23, 25, 29, 30-35, 44, 45, 48, 60, 62, 64, 65, 66, 68, and 74. In some embodiments, the compound is selected from 11, 20, 21, 31, 68, and 74. In some embodiments, the compound is other than 9, 11, or 14.

An effective amount of the compounds disclosed herein comprise amounts sufficient to produce a noticible effect, such as, but not limited to, a reduction or cessation of self-administration of alcohol or another substance of abuse, weight loss, lack of weight gain, etc.). Actual dosage levels of active ingredients in a therapeutic compound of the presently disclosed subject matter can be varied so as to administer an amount of the active compound that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

The therapeutically effective amount of a compound can depend on a number of factors. For example, the species, age, and weight of the subject, the precise condition requiring treatment and its severity, the nature of the formulation, and the route of administration are all factors that can be considered. In some embodiments, the therapeutically effective amount is in the range of about 0.1 to about 100 mg/kg body weight of the subject per day. In some embodiments, the therapeutically effective amound is in the range of from about 0.1 to about 20 mg/kg body weight per day. Thus, for a 70 kg adult mammal, one example of an actual amount per day would be between about 10 and about 2000 mg. This amount can be given in a single dose per day or in a number (e.g., 2, 3, 4, or 5) of sub-doses per day such that the total daily dose is the same. The effective amount of a salt or solvate thereof can be determined as a proportion of the effective amount of the compound per se.

A compound of the presently disclosed subject matter can also be useful as adjunctive, add-on or supplementary therapy for the treatment of the above-mentioned diseases/disorders. Said adjunctive, add-on or supplementary therapy means the concomitant or sequential administration of a compound of the presently disclosed subject matter to a subject who has already received administration of, who is receiving administration of, or who will receive administration of one or more additional therapeutic agents for the treatment of the indicated conditions, for example, one or more known anti-depressant, anti-psychotics or anxiolytic agents.

In some embodiments, the presently disclosed subject matter provides a compound of Formula (I), (Ia), (II), (IIa), (III) or (IIIa) for use as an active therapeutic substance. In some embodiments, the compound is for use in the treatment of a disease mediated by CB1R. In some embodiments, the presently disclosed subject matter provides the use of the compound of Formula (I), (Ia), (II), (IIa), (III), or (IIIa) for the preparation of a medicament for the treatment of a disease mediated by CB1R.

In some embodiments, the presently disclosed subject matter provides a method of modulating the activity of CB1R, wherein the method comprises contacting a sample comprising CB1R with a compound of one of Formula (I), (Ia), (II), (IIa), (III), or (IIIa), or a pharmaceutically acceptable salt or solvate thereof, or a pharmaceutical composition thereof. In some embodiments, the sample is an ex vivo sample. In some embodiments, the sample comprises a biological fluid, e.g. plasma, cerebrospinal fluid, saliva. In some embodiments, the sample comprises an organ, tissue, cell or cell extract. In some embodiments, the sample is from a subject. In some embodiments, the method can further comprises contacting the sample with second compound, such as a compound having or suspected of having CB1R agonist or antagonist activity.

### VI. Methods of Preparing Urea Derivatives

The presently disclosed antagonists can be prepared using standard synthetic methodology known in the art. For example, the compounds can be made by the methods described hereinbelow or variations thereof that will be apparent to persons skilled in the art based on the present disclosure. As necessary, protecting groups known in the art can be utilized during the synthesis of the compounds.

Generally, the presently disclosed urea-based compounds can be prepared by coupling of an amine (e.g., representing the right side of the compound of Formula (I), i.e., representing the L₁ and R₆ groups) with an isocyanate (e.g., representing the left side of the molecule. For example, several of the presently disclosed compounds can be prepared by coupling an amine derivative of the Li-R₆ group of Formula (I) with 4-chlorophenyl isocyanate. Alternatively, the ureas can be prepared via Curtius reaction of an amine and an acyl azide, which can itself be prepared by reacting an acid chloride or anhydride with sodium azide or trimethylsilyl azide. In some embodiments, the amine representing the right side of the urea can be purchased from a commercial source. In some embodiments, the amine can be prepared, for example, via a Suzuki coupling reaction (e.g., wherein an aryl halide is reacted with an aryl boronic acid in the presence of a Pd(0) catalyst, such as Pd(PPh3)4) or another suitable coupling reaction of precursors of the

L₁ and R₆ groups of the compound of Formula (I). In some embodiments, one of the coupling partners comprises a nitro group that can be reduced after the coupling reaction with a suitable reducing agent (e.g., Raney nickel) to provide an amino group. Schemes 3-7, below, show the synthesis of compounds of Formula (I) with different L₁ groups. As would be understood by one of ordinary skill in the art, these schemes can be adapted to prepare additional compounds by using other starting materials. More particularly, compound 6, an exemplary compound of Formula (I) wherein L₁ is substituted arylene, was prepared as shown in Scheme 3, below, from 2-bromo-6-(pyrrolidin-1-yl)pyridine. See German et al., J. Med. Chem. 2014, 57, 7758-7769) 2-Bromo-6-(pyrrolidin-1-yl)pyridine underwent Suzuki coupling with 4-methoxy-3-nitro-phenylboronic acid to give nitro compound 85 which was subsequently reduced by Raney-nickel and hydrazine to amine 86.

Urea 6 was then obtained by coupling of 86 with 4-chlorophenyl isocyanate. Additional compounds with central substituted phenyl rings can be prepared by substituting the 4-methoxy-3-nitro-phenylboronic acid for another nitro-phenylboronic acid and/or by substituteing the 2-bromo-6-(pyrrolidin-1-yl)pyridine with another aryl halide.

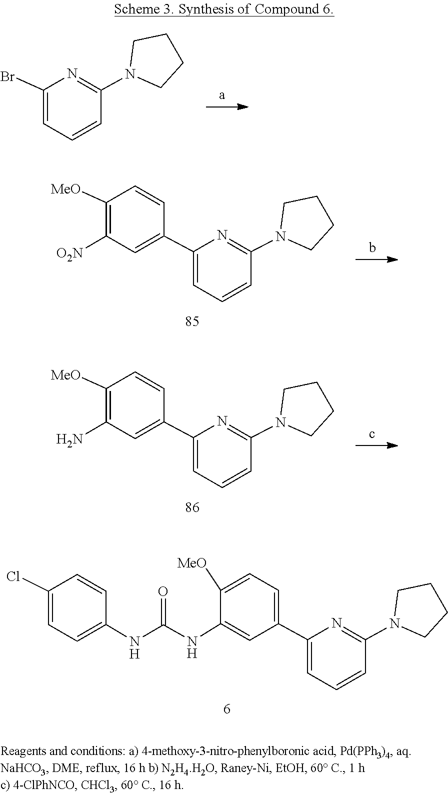 [SMILES: Brc1cccc(N2CCCC2)n1 ; COc1ccc(-c2cccc(N3CCCC3)n2)cc1[N+](=O)[O-] ; COc1ccc(-c2cccc(N3CCCC3)n2)cc1N ; COc1ccc(-c2cccc(N3CCCC3)n2)cc1NC(=O)Nc1ccc(Cl)cc1]

Compounds 7-10, which include a central pyridinyl ring were prepared as shown in Scheme 4, below. As shown in Scheme 4, the corresponding bromopyridinamines underwent Suzuki coupling with phenylboronic acid to give intermediates 87-90 which were subsequently coupled with 4-chlorophenyl isocyanate to afford compounds 7-10. Additional compounds with central pyridinyl rings can be made in an analogous manner using other substituted phenyl boronic acids and/or further substituted bromo-aminopyridines.

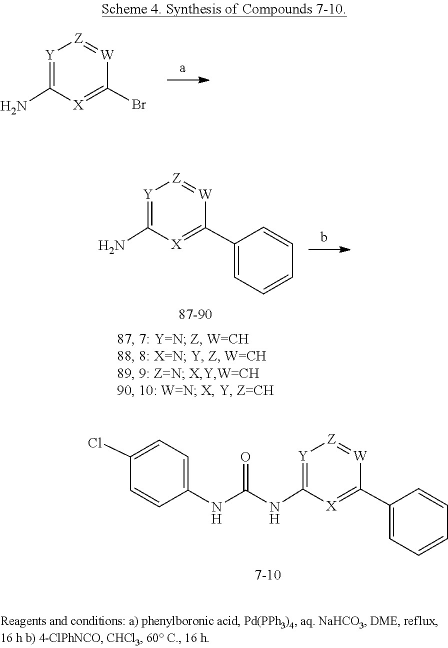

Thiophenyl- and cyclopropyl-containing compounds can be prepared according to routes similar to those shown for exemplary compounds 12, 13, 15, and 16 in Scheme 5, below. As shown in Scheme 5, bromothiophenecarboxylic acids underwent Suzuki coupling with phenylboronic acid to afford the intermediates 91 and 92. The final products 12 and 13 was obtained via a microwave-assisted coupling of carboxylic acids 91 and 92 respectively via a Curtius rearrangement (see Kulkarni et al., J. Org. Chem. 2017, 82, 992-999) with 4-chlorophenylamine in the presence of diphenylphosphoryl azide. Similarly, cyclopropenyl compounds 15 and 16 were afforded from the Curtius rearrangement reaction of cis- or trans-2-phenylcyclopropane-1-carboxylic acid and 4-chlorophenylamine correspondingly. Again, additional compounds with central thiophenyl or cyclopropenyl groups can be prepared by using substituted phenyl boronic acids and/or other halophenylamines.

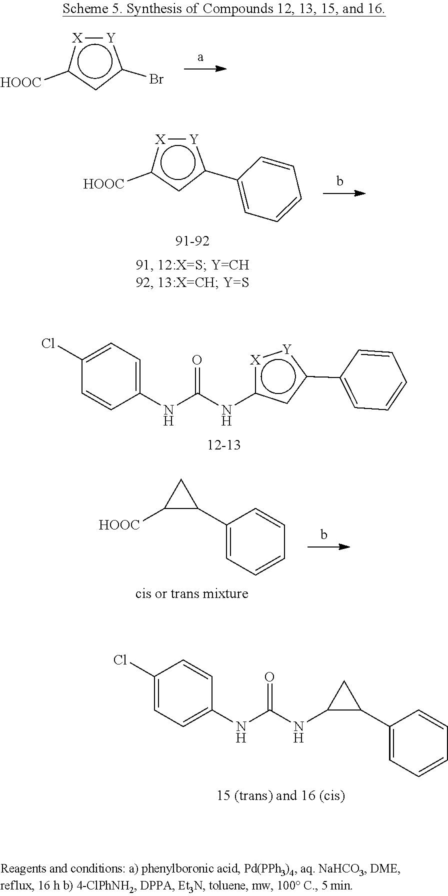

The Suzuki coupling between 2-bromo-5-nitrothiazole and phenylboronic acid failed to give thiazole intermediate 93. Thus, a different route was sought to form the thiazole ring of compounds with a central thiazole group. Compounds with a central thiazole group can be prepared in a manner similar to that shown for exemplary thiazole compound 14. See Scheme 6, below. Phenyl acetaldehyde was treated with bromine to give 2-bromo-2-phenylacetaldehyde which underwent cyclization with thiourea to give the intermediate 93. See Guo and Yan, Eur. J. lnorg. Chem. 2010, 1267-1274. Coupling of 93 with 4-chlorophenyl isocyanate yielded 14. Additional thiazole-contianing compounds can be prepared by substituting the phenyl acetaldehyde with an aryl-substituted phenyl acetaldehyde.

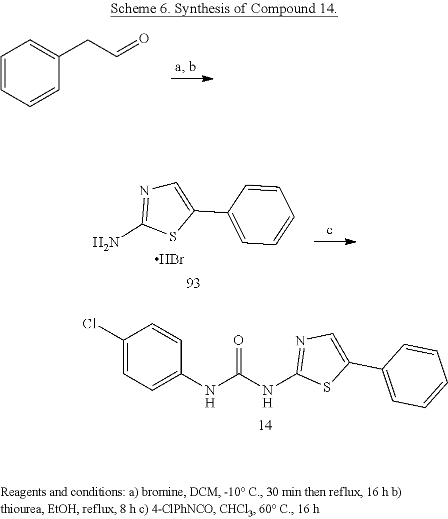 [SMILES: O=CCc1ccccc1 ; Nc1ncc(-c2ccccc2)s1 ; Br ; O=C(Nc1ccc(Cl)cc1)Nc1ncc(-c2ccccc2)s1]

Piperidine compounds can be prepared as shown in Scheme 7, below, for exemplary piperidine compound 17. Copper-catalyzed coupling between (R)-3-(Boc-amino)piperidine and phenylboronic acid yielded the intermediate 94.Removal of the Boc protecting group resulted in amine 95 which subsequently underwent coupling with 4-chlorophenyl isocyanate afforded compound 17.

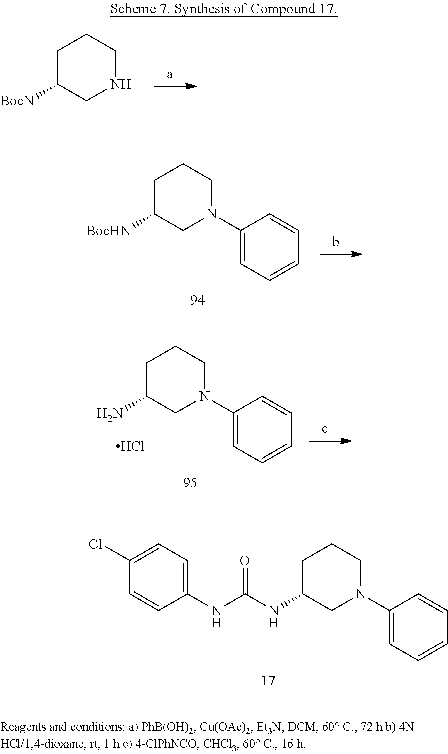

The standard procedure utilized to prepare the 5-phenyl-thiophen-2-yl analogues is shown in Scheme 8, below, starting with Suzuki coupling between 2-bromo-5-nitrothiophene and a substituted phenylboronic acid to give nitro intermediates 96-113 and 140, which were reduced by Raney-Ni and hydrazine to amines 114-131 and 141. Coupling of these amines with 4-chlorophenyl isocyanate afforded compounds 11, and 18-35.

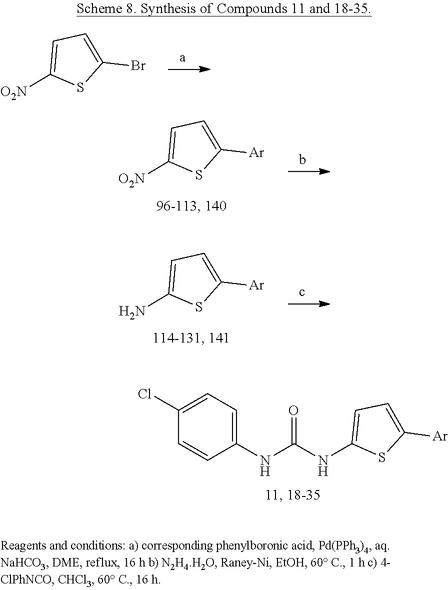

Compounds where the central ring is replaced by an aliphatic group, e.g., exemplary compounds 36-39 and 41-84, were obtained by coupling the corresponding aliphatic primary amines to 4-chlorophenyl isocyanate, as shown in Scheme 9, below. The primary amines were either purchased from commercial vendors or prepared as depicted in Scheme 9. Carboxamide 132 was prepared from the amide coupling of the trans-2-phenylcyclopropane-1-carboxylic acid with ammonia. Amines 133-135 were prepared from the reduction of corresponding benzonitrile or carboxamide by borane dimethyl sulfide. Alternately, amines 136 and 137 were obtained from the reduction of the corresponding substituted benzonitrile by LiA11-14 in THF. To prepare 40, the alkylation of the enolate anion of methyl isobutyrate with benzyl bromide afforded the intermediate 138 which was hydrolyzed to give acid 139 which underwent a microwave-assisted coupling via a Curtius rearrangement (see Kulkarni et al., J. Org. Chem. 2017, 82, 992-999) with 4-chlorophenylamine in the presence of diphenylphosphoryl azide to give compound 40.

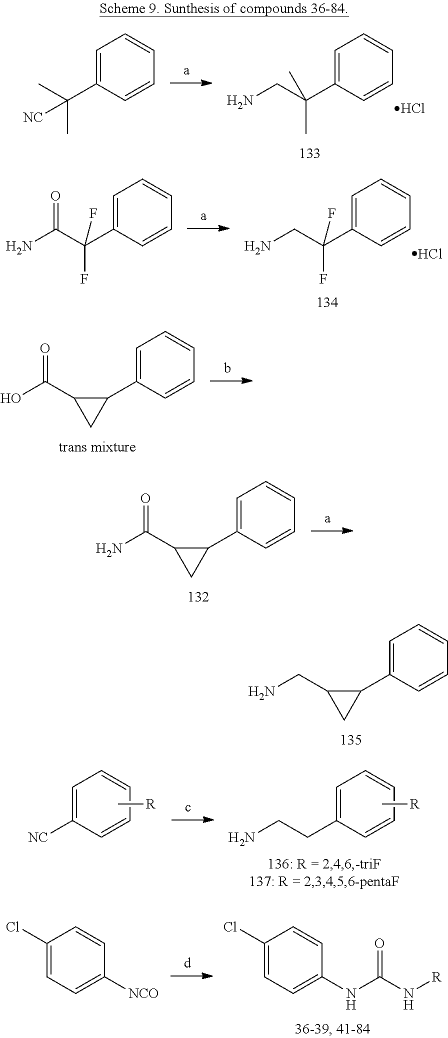

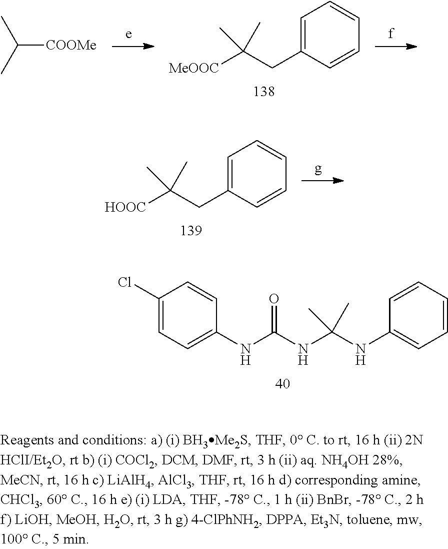

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following

Examples are intended to be exemplary only and that numerous changes, modifications, and alterations can be employed without departing from the scope of the presently disclosed subject matter.

### Example 1

### Synthesis of CB1 Allosteric Modulators

All solvents and chemicals were reagent grade. Unless otherwise mentioned, all reagents and solvents were purchased from commercial vendors and used as received. Flash column chromatography was carried out on a Teledyne ISCO COMBIFLASHTM Rf system (Teledyne ISCO Co.,

Lincoln, Nebraska, United States of America) using prepacked columns. Solvents used include hexanes, ethyl acetate (EtOAc), dichloromethane, and methanol. Purity and characterization of compounds were established by a combination of high pressure liquid chromatography (HPLC), thin layer chromatography (TLC), mass spectrometry (MS), and nuclear magnetic resonance (NMR) analyses. ¹H and ¹³C NMR spectra were recorded on a Bruker Avance DPX-300 (300 MHz) spectrometer (Bruker Corporation, Billerica, Massachusetts, United States of America) and were determined in CDC13, DMSO-d6, or CD3OD with tetramethylsilane (TMS) (0.00 ppm) or solvent peaks as the internal reference. Chemical shifts are reported in ppm relative to the reference signal, and coupling constant (J) values are reported in hertz (Hz). TLC was performed on EMD precoated silica gel 60 F254 plates (MilliporeSigma, Merck KGH, Darmstadt, Germany), and spots were visualized with UV light or iodine staining. Nominal mass spectra were obtained using an Agilent 1260 Infinity II system (electrospray ionization (ESI)) (Agilent Technologies, Santa Clara, Calif., United States of America). High resolution mass spectra were obtained using Agilent 1290 Infinity UHPLC-6230 TOF system (ESI) (Agilent Technologies, Santa Clara, Calif., United States of America). All final compounds were greater than 95% pure as determined by HPLC on an Agilent 1100 system using an Agilent ZORBAXTM SB-Phenyl, 2.1 mm x 150 mm, 5 μm column (Agilent Technologies, Santa Clara, Calif., United States of America) using a 15 minute gradient elution of 5-95% solvent B at 1 mL/min followed by 10 minutes at 95% solvent B (solvent A, water with 0.1% TFA; solvent B, acetonitrile with 0.1% TFA and 5% water; absorbance monitored at 220 and 280 nm). General procedure A. To a mixture of aryl bromide (1 eq), boronic acid (1.1 eq) in dimethoxyethane (0.1 M) was added 1M aqueous NaHCO3 solution (3 eq) followed by Pd(Ph3)4 (0.075 eq). The reaction mixture was refluxed overnight under nitrogen atmosphere. The reaction mixture was diluted with ethyl acetate, washed with a saturated NaHCO3 solution and brine. The combined organic layers were dried over anhydrous MgSO4 and filtered. The filtrate was concentrated in vacuo and the residue was purified by column chromatography (SiO2, ethyl acetate/hexanes) to give the desired product.

2-(4-Methoxy-3-nitrophenyl)-6-(pyrrolidin-1-yl)pyridine (85) was prepared from 2-bromo-6-(pyrrolidin-1-yl)pyridine (0.30 g, 1.32 mmol; German et al., J. Med. Chem. 2014, 57, 7758-7769) and 4-methoxy-3-nitrophenylboronic acid (0.29 g, 1.45 mmol) following the general procedure A as yellow solid (0.12 g, 30%). ¹H NMR (300 MHz, CDC13) δ 8.23 (dd, J=1.22, 8.76 Hz, 1H), 7.46-7.56 (m, 1H), 7.13 (d, J=8.85 Hz, 1H), 6.91-7.00 (m, 2H), 6.34 (d, J=8.48 Hz, 1 H), 3.54 (t, J=6.50 Hz, 4H), 1.99-2.07 (m, 4H). MS (ESI) m/z [M+H]+calcd: 300.1; found: 300.4.

4-Phenylpyridin-2-amine (87) was prepared from phenylboronic acid (0.10 g, 0.58 mmol) and 4-bromopyridin-2-amine (0.08 g, 0.64 mmol) following the general procedure A as white solid (0.09 g, 89%). ¹H NMR (300 MHz, CDC13) δ 8.12 (d, J=5.27 Hz, 1H), 7.62-7.73 (m, 2H), 7.51-7.61 (m, 3H), 7.38-7.49 (m, 5H), 6.88 (d, J=5.27 Hz, 1H), 6.70 (s, 1H), 4.57 (br. s., 2H). MS (ESI) m/z [M+H]+calcd: 171.1; found: 171.1.

6-Phenylpyridin-2-amine (88) was prepared from 6-bromopyridin-2-amine (0.10 g, 0.58 mmol) and phenyl boronic acid (0.08 g, 0.64 mmol) following the general procedure A as yellow liquid (0.10 g, 79%). ¹H NMR (300 MHz, CDC13) δ 7.92 (dd, J=1.22, 8.19 Hz, 2H), 7.32-7.52 (m, 4H), 7.07 (d, J=7.35 Hz, 1 H), 6.43 (d, J=8.10 Hz, 1 H), 4.55 (br. s., 2H). MS (ESI) m/z [M+H]+calcd: 171.1; found: 171.2.

5-Phenylpyridin-3-amine (89) was prepared from 5-bromopyridin-3-amine (0.10 g, 0.58 mmol) and phenyl boronic acid (0.08 g, 0.64 mmol) following the general procedure A as white solid (0.10 g, 60%). ¹H NMR (300 MHz, CDC13) δ 8.24 (d, J=1.70 Hz, 1 H), 8.06 (d, J=2.45 Hz, 1 H), 7.62-7.71 (m, 3H), 7.42 −7.47 (m, 3H), 7.15 (dd, J=1.88, 2.64 Hz, 1H), 3.89 (br. s., 2H). MS (ESI) m/z [M+H]+calcd: 171.1; found: 171.0.

2-Phenylpyridin-4-amine (90) was prepared from 2-bromopyridin-4-amine (0.10 g, 0.58 mmol) and phenyl boronic acid (0.08 g, 0.64 mmol) following the general procedure A as yellow liquid (0.04 g, 44%). ¹H NMR (300 MHz, CDC13) δ 8.30 (d, J=5.46 Hz, 1 H), 7.91 (s, 2H), 7.32-7.49 (m, 3H), 6.93 (s, 1 H), 6.47 (dd, J=2.17, 5.56 Hz, 1 H), 4.25 (br. s., 2H). MS (ESI) m/z[M+H]⁻ calcd: 171.1; found: 171.2.

2-Nitro-5-phenylthiophene (140) was prepared from 2-bromo-5-nitrothiophene (0.20 g, 0.96 mmol) phenyl boronic acid (0.13 g, 1.06 mmol) following the general procedure A as yellow liquid (0.06 g, 28%). ¹H NMR (300 MHz, CDC13): δ 7.91 (d, J=4.10 Hz, 1H), 7.61-7.64 (m, 2H), 7.42-7.48 (m, 3H), 7.24 (d, J=5.20 Hz, 1 H) ppm. MS (ESI) m/z [M-H]⁻ calcd: 203.1; found:

203.3.

2-(4-Fluorophenyl)-5-nitrothiophene (96) was prepared from 2-bromo-5-nitrothiophene (0.20 g, 0.96 mmol) and 4-fluorophenylboronic acid (0.15 g, 1.06 mmol) following the general procedure A as white solid (0.03 g, 12%). ¹H NMR (300 MHz, CDC13) δ 7.90 (d, J=4.33 Hz, 1H), 7.62 (dd, J=5.18, 8.76 Hz, 2H), 7.11-7.21 (m, 3H).

2-(3-Fluorophenyl)-5-nitrothiophene (97) was prepared from 2-bromo-5-nitrothiophene (0.21 g, 1 mmol) and 3-fluorophenylboronic acid (0.15 g, 1.1 mmol) following the general procedure A as yellow solid (0.10 g, 45%). ¹H NMR (300 MHz, CDC13) δ 7.91 (d, J=4.33 Hz, 1 H), 7.39-7.49 (m, 2H), 7.29-7.36 (m, 1H), 7.25 (d, J=4.33 Hz, 1 H), 7.09-7.18 (m, 1H).

2-(2,4-Difluorophenyl)-5-nitrothiophene (98) was prepared from 2-bromo-5-nitrothiopene (0.21 g, 1 mmol) and 2,4-f luorophenylboronic acid (0.20 g, 1.0 mmol) following the general procedure A as yellow solid (0.07 g, 29%). ¹H NMR (300 MHz, CDC13) δ 7.93 (d, J=0.75 Hz, 1 H), 7.64 (dt, J=6.03, 8.85 Hz, 1 H), 7.34 (d, J=4.33 Hz, 1 H), 6.93-7.05 (m, 2H).

2-(2-Chlorophenyl)-5-nitrothiophene (99) was prepared from 2-bromo-5-nitrothiopene (0.21 g, 1 mmol) and 2-chlorophenylboronic acid (0.21 g, 1.0 mmol) following the general procedure A as yellow solid (0.08 g, 33%). ¹H NMR (300 MHz, CDC13) δ 7.92 (d, J=4.33 Hz, 1 H), 7.50-7.57 (m, 2H), 7.34-7.40 (m, 2H), 7.29 (d, J=4.33 Hz, 1 H).

2-(3-Chlorophenyl)-5-nitrothiophene (100) was prepared from 2-bromo-5-nitrothiopene (0.21 g, 1 mmol) and 3-chlorophenylboronic acid (0.17 g, 1.0 mmol) following the general procedure A as yellow solid (0.09 g, 38%). ¹H NMR (300 MHz, CDC13) δ 7.91 (d, J=4.33 Hz, 1 H), 7.59-7.63 (m, 1 H), 7.48-7.53 (m, 1H), 7.38-7.44 (m, 2H), 7.23-7.27 (m, 1H).

2-(4-Chlorophenyl)-5-nitrothiophene (101) was prepared from 2-bromo-5-nitrothiopene (0.21 g, 1 mmol) and 4-chlorophenylboronic acid (0.17 g, 1.0 mmol) following the general procedure A as yellow solid (0.09 g, 38%). ¹H NMR (300 MHz, CDC13) δ 7.91 (d, J=4.33 Hz, 1 H), 7.54-7.59 (m, 2H), 7.41-7.46 (m, 2H), 7.22 (d, J=4.33 Hz, 1 H).

2-(3,4-Dichlorophenyl)-5-nitrothiophene (102) was prepared from 2-bromo-5-nitrothiopene (0.20 g, 1 mmol) and 2,4-dichlorophenylboronic acid (0.20 g, 1.1 mmol) following the general procedure A as yellow solid (0.02 g, 8%). ¹H NMR (300 MHz, CDC13) δ 7.91 (d, J=4.33 Hz, 1 H), 7.72 (d, J=2.07 Hz, 1H), 7.55 (s, 1H), 7.43-7.47 (m, 1 H), 7.24 (d, J=4.33 Hz, 1 H).

2-(3,5-Dichlorophenyl)-5-nitrothiophene (103) was prepared from 2-bromo-5-nitrothiopene (0.21 g, 1 mmol) and 3,5-dichlorophenylboronic acid (0.21 g, 1.1 mmol) following the general procedure A as yellow solid (0.05 g, 16%). ¹H NMR (300 MHz, CDC13) δ 7.89-7.94 (m, 1 H), 7.50 (br. s., 2H), 7.40-7.45 (m, 1 H), 7.24-7.29 (m, 1H). 1-[3-(5-Nitrothiophen-2-yl)phenyl]ethan-1-one (104) was prepared from 2-bromo-5-nitrothiopene (0.21 g, 1 mmol) and 3-(methoxycarbonyl)phenylboronic acid (0.20 g, 1.1 mmol) following the general procedure A as yellow solid (0.09 g, 40%). ¹H NMR (300 MHz, CDC13) δ 8.22 (s, 1H), 8.00 (d, J=6.59 Hz, 1H), 7.91-7.96 (m, 1H), 7.77-7.86 (m, 1H), 7.53-7.63 (m, 1H), 7.23-7.37 (m, 2H), 2.67 (s, 3H). Methyl 3-(5-nitrothiophen-2-yl)benzoate (105) was prepared from 2-bromo-5-nitrothiopene (0.21 g, 1 mmol) and 3-acetoxyphenylboronic acid (0.18 g, 1.1 mmol) following the general procedure A as red solid (0.09 g, 33%). ¹H NMR (300 MHz, CDC13) δ 8.30 (s, 1H), 8.10 (d, J=7.91 Hz, 1H), 7.91-7.96 (m, 1H), 7.81 (d, J=7.91 Hz, 1H), 7.51-7.59 (m, 1H), 7.33 (d, J=4.33 Hz, 1H), 3.97 (s, 3H). 2-(3-Methanesulfonylphenyl)-5-nitrothiophene (106) was prepared from 2-bromo-5-nitrothiopene (0.21 g, 1 mmol) and 3-(methylsulfonyl)phenylboronic acid (0.22 g, 1.1 mmol) following the general procedure A as red solid (0.08 g, 28%). ¹H NMR (300 MHz, CDC13) δ 8.20 (t, J=1.60 Hz, 1H), 8.01 (d, J=7.91 Hz, 1 H), 7.95 (d, J=4.33 Hz, 1 H), 7.90 (d, J=7.91 Hz, 1 H), 7.66-7.73 (m, 1 H), 7.37 (d, J=4.33 Hz, 1 H), 3.12 (s, 3H).

2-(2-Methoxyphenyl)-5-nitrothiophene (107) was prepared from 2-bromo-5-nitrothiopene (0.21 g, 1 mmol) and 2-methoxyphenylboronic acid (0.17 g, 1.1 mmol) following the general procedure A as yellow solid (0.20 g, 85%). ¹H NMR (300 MHz, CDC13) δ 7.90 (d, J=4.52 Hz, 1 H), 7.73 (dd, J=1.22, 7.82 Hz, 1 H), 7.38-7.44 (m, 2H), 7.01-7.11 (m, 2H), 4.01 (s, 3H).

2-(3-Methoxyphenyl)-5-nitrothiophene (108) was prepared from 2-bromo-5-nitrothiopene (0.21 g, 1 mmol) and 3-methoxyphenylboronic acid (0.17 g, 1.1 mmol) following the general procedure A as yellow solid (0.15 g, 66%). ¹H NMR (300 MHz, CDC13) δ 7.90 (d, J=4.33 Hz, 1 H), 7.31-7.35 (m, 1 H), 7.18-7.25 (m, 2H), 7.13 (t, J=1.98 Hz, 1 H), 6.98 (dd, J=1.98, 8.19 Hz, 1H), 3.87 (s, 3H).

2-(4-Methoxyphenyl)-5-nitrothiophene (109) was prepared from 2-bromo-5-nitrothiopene (0.21 g, 1 mmol) and 4-methoxyphenylboronic acid (0.17 g, 1.1 mmol) following the general procedure A as yellow solid (0.15 g, 80%). ¹H NMR (300 MHz, CDC13) δ 7.85-7.91 (m, 1 H), 7.57 (d, J=8.10 Hz, 2H), 7.10-7.17 (m, 1H), 6.97 (d, J=6.41 Hz, 2H), 3.86 (s, 3H).

2-(3-Methylphenyl)-5-nitrothiophene (110) was prepared from 2-bromo-5-nitrothiopene (0.21 g, 1 mmol) and 3-methylphenylboronic acid (0.17 g, 1.1 mmol) following the general procedure A as yellow solid (0.08 g, 35%). ¹H

NMR (300 MHz, CDC13) δ 7.90 (d, J=4.33 Hz, 1 H), 7.41-7.46 (m, 2H), 7.34 (t, J=7.82 Hz, 1 H), 7.21-7.27 (m, 2H), 2.42 (s, 3H). N,N-Dimethyl-3-(5-nitrothiophen-2-yl)aniline (111) was prepared from 2-bromo-5-nitrothiopene (0.21 g, 1 mmol) and 3-(N,N-dimethylamino)phenylboronic acid (0.18 g, 1.1 mmol) following the general procedure A as orange solid (0.06 g, 26%). ¹H NMR (300 MHz, CDC13) δ 7.87 (d, J=4.33 Hz, 1 H), 7.25-7.32 (m, 1H), 7.21 (d, J=4.33 Hz, 1H), 6.95 (d, J=7.72 Hz, 1H), 6.86 (s, 1H), 6.78 (dd, J=1.88, 8.29 Hz, 1H), 3.01 (s, 6H). 3-(5-Nitrothiophen-2-yl)pyridine (112) was prepared from 2-bromo-5-nitrothiopene (0.21 g, 1 mmol) and 3-pyridylboronic acid (0.14 g, 1.1 mmol) following the general procedure A as orange solid (0.07 g, 34%). ¹H NMR (300 MHz, CDC13) δ 8.92 (d, J=1.88 Hz, 1 H), 8.68 (d, J=3.96 Hz, 1H), 7.89-7.97 (m, 2H), 7.42 (dd, J=4.90, 7.91 Hz, 1H), 7.32 (d, J=4.14 Hz, 1H). MS (ESI) m/z [M+H]+calcd: 207.1; found: 207.1. 4-(5-Nitrothiophen-2-yl)pyridine (113) was prepared from 2-bromo-5-nitrothiopene (0.21 g, 1 mmol) and 4-pyridylboronic acid (0.14 g, 1.1 mmol) following the general procedure A as yellow solid (0.07 g, 17%). ¹H NMR (300 MHz, CDC13) δ 8.69-8.76 (m, 2H), 7.95 (d, J=4.14 Hz, 1H), 7.48-7.54 (m, 2H), 7.43 (d, J=4.33 Hz, 1 H). MS (ESI) m/z[M+H]+calcd: 207.1; found: 207.2. General procedure B. To a solution of nitrobenzene derivative (1 eq) in ethanol (0.1 M) was added hydrazine hydrate (15 eq). The reaction was stirred at 50° C. for 15 min and an excess of Raney nickel slurry in water (1.2 eq) was added slowly. After 1 h, the bubbling ceased, the mixture was cooled to room temperature and filtered through Celite. The filtrate was condensed under reduced pressured and the residue was either used for the next step without purification or purified by column chromatography (SiO2, ethyl acetate/hexanes) to afford the desired product. 2-Methoxy-5-[6-(pyrrolidin-1-yl)pyridin-2-yl]aniline (86) was prepared from 85 (0.12 g, 0.4 mmol) following the general procedure B as white solid (0.10 g, 93%). ¹H NMR (300 MHz, CDC13) δ 7.49 (d, J=2.07 Hz, 1H), 7.44-7.48 (m, 1H), 7.39-7.43 (m, 1H), 6.92 (d, J=7.54 Hz, 1H), 6.83 (d, J=8.48 Hz, 1 H), 6.25 (d, J=8.29 Hz, 1 H), 3.89 (s, 3H), 3.84 (br. s., 2H), 3.54 (t, J=6.59 Hz, 4H), 2.00 (t, J=6.59 Hz, 4H). MS (ESI) m/z [M+H]+calcd: 270.1; found: 270.3.

5-Phenylthiophen-2-amine (141) was prepared from 140 (0.06 g, 0.3 mmol) following the general procedure B as white solid (0.05 g, 96%). ¹H NMR (300 MHz, CDC13) δ 7.46 (s, 2H), 7.31 (d, J=15.26 Hz, 2H), 7.18 (d, J=7.54 Hz, 1H), 6.93 (s, 1H), 6.15 (d, J=3.77 Hz, 1H), 3.82 (br. s., 2H). MS (ESI) m/z [M+H]+calcd: 176.1; found: 176.1.

5-(4-Fluorophenyl)thiophen-2-amine (114) was prepared from 96 (0.03 g, 0.12 mmol) following the general procedure B as white solid (0.01 g, 36%). ¹H NMR (300 MHz, CDC13) δ 7.34-7.46 (m, 2H), 6.95-7.07 (m, 2H), 6.83 (d, J=2.45 Hz, 1H), 6.11-6.19 (m, 1H), 3.82 (br. s., 2H). MS (ESI) m/z [M+H]+calcd: 194.1; found: 194.2.

5-(3-Fluorophenyl)thiophen-2-amine (115) was prepared from 97 (0.10 g, 0.45 mmol) following the general procedure B as white solid (0.07 g, 82%). ¹H NMR (300 MHz, CDC13) δ 7.24 (d, J=9.42 Hz, 2H), 7.14 (d, J=9.80 Hz, 1H), 6.91-7.00 (m, 1H), 6.80-6.90 (m, 1H), 6.10-6.20 (m, 1H), 3.88 (br. s., 2H). MS (ESI) m/z [M+H]+calcd: 194.1; found: 194.3.

5-(2,4-Difluorophenyl)thiophen-2-amine (116) was prepared from 98 (0.07 g, 0.68 mmol) following the general procedure B as white solid (0.03 g, 49%). ¹H NMR (300 MHz, CDC13) δ 7.41 (dt, J=6.41, 8.57 Hz, 15H), 7.01 (dd, J=1.13, 3.77 Hz, 14H), 6.79-6.90 (m, 29H), 6.17 (d, J=3.77 Hz, 14H), 3.88 (br. s., 28H). MS (ESI) m/z [M+H]+calcd: 212.1; found: 212.1.

5-(2-Chlorophenyl)thiophen-2-amine (117) was prepared from 99 (0.08 g, 0.33 mmol) following the general procedure B as white solid (0.04 g, 56%). ¹H NMR (300 MHz, CDC13) δ 7.45 (dd, J=1.51, 7.72 Hz, 1 H), 7.41 (dd, J=1.32, 7.72 Hz, 1H), 7.19-7.24 (m, 1H), 7.16 (dd, J=1.51, 7.54 Hz, 1H), 7.03 (s, 1H), 6.19 (d, J=3.77 Hz, 1H), 3.87 (br. s., 2H). MS (ESI) m/z [M+H]+calcd: 210.1; found: 210.1.

5-(3-Chlorophenyl)thiophen-2-amine (118) was prepared from 100 (0.09 g, 0.38 mmol) following the general procedure B as white solid (0.04 g, 20%). ¹H NMR (300 MHz, CDC13) δ 7.42 (t, J=1.70 Hz, 1H), 7.28-7.34 (m, 1H), 7.19-7.24 (m, 1H), 7.09-7.16 (m, 1H), 6.94 (d, J=3.77 Hz, 1H), 6.15 (d, J=3.77 Hz, 1 H), 3.89 (br. s., 2H). MS (ESI) m/z [M+H]+calcd: 210.0; found: 210.1.

5-(4-Chlorophenyl)thiophen-2-amine (119) was prepared from 101 (0.10 g, 0.40 mmol) following the general procedure B as white solid (0.03 g, 29%). ¹H NMR (300 MHz, CDC13) δ 7.33-7.40 (m, 2H), 7.23-7.30 (m, 2H), 6.90 (d, J =3.77 Hz, 1H), 6.15 (d, J=3.58 Hz, 1H), 3.86 (br. s., 2H). MS (ESI) m/z [M+H]⁻ calcd: 210.0; found: 210.2.

5-(2,4-Dichlorophenyl)thiophen-2-amine (120) was prepared from 102 (0.02 g, 0.07 mmol) following the general procedure B as white solid (0.01 g, 36%). ¹H NMR (300 MHz, CDC13) δ 7.50 (d, J=2.07 Hz, 1H), 7.33-7.39 (m, 1H), 7.23 (d, J=2.26 Hz, 1H), 6.92 (d, J=3.77 Hz, 1H), 6.15 (d, J=3.77 Hz, 1 H), 3.91 (br. s., 2H). MS (ESI) m/z [M+H]+calcd: 244.0; found: 244.0.

5-(3,5-Dichlorophenyl)thiophen-2-amine (121) was prepared from 103 (0.05 g, 0.16 mmol) following the general procedure B as light yellow solid (0.01 g, 38%). ¹H NMR (300 MHz, CDC13) δ 7.29 (d, J=1.70 Hz, 2H), 7.13 (s, 1H), 6.95 (d, J=3.77 Hz, 1H), 6.14 (d, J=3.77 Hz, 1H), 3.95 (br. s., 2H). MS (ESI) m/z [M+H]+calcd: 244.0; found: 244.1.

1-[3-(5-Aminothiophen-2-yl)phenyl]ethan-1-one (122) was prepared from 104 (0.10 g, 0.40 mmol) following the general procedure B as yellow solid (0.06 g, 68%). ¹H NMR (300 MHz, CDC13) δ 7.99-8.05 (m, 1H), 7.74 (d, J=6.40 Hz, 1H), 7.63 (d, J=5.84 Hz, 1H), 7.41 (d, J=7.16 Hz, 1H), 6.96-7.05 (m, 1H), 6.13-6.24 (m, 1H), 3.91 (br. s., 2H), 2.62 (s, 3H). MS (ESI) m/z

[M+H]⁻ calcd: 218.1; found: 218.2. Methyl 3-(5-aminothiophen-2-yl)benzoate (123) was prepared from 105 (0.09 g, 0.33 mmol) following the general procedure B as yellow solid (0.04 g, 49%). ¹H NMR (300 MHz, CDC13) δ 8.09-8.16 (m, 1H), 7.83 (d, J=6.22 Hz, 1H), 7.57-7.67 (m, 1H), 7.32-7.44 (m, 1H), 6.98-7.05 (m, 1H), 6.13-6.21 (m, 1 H), 3.93 (s., 3H), 3.82 (br. s., 2H). MS (ESI) m/z [M+H]+calcd: 234.1; found: 234.3. 5-(3-Methanesulfonylphenyl)thiophen-2-amine (124) was prepared from 106 (0.08 g, 0.28 mmol) following the general procedure B as yellow solid (0.04 g, 54%). ¹H NMR (300 MHz, CDC13) δ 7.98 (t, J=1.79 Hz, 1 H), 7.64-7.73 (m, 2H), 7.45-7.54 (m, 1H), 7.05 (d, J=3.77 Hz, 1H), 6.17 (d, J=3.77 Hz, 1 H), 3.99 (br. s., 2H), 3.07 (s, 3H). MS (ESI) m/z [M+H]+calcd: 254.1; found: 254.3.

5-(2-Methoxyphenyl)thiophen-2-amine (125) was prepared from 107 (0.20 g, 0.85 mmol) following the general procedure B as white solid (0.04 g, 18%). ¹H NMR (300 MHz, CDC13) δ 7.51 (dd, J=1.51, 7.72 Hz, 1H), 7.09-7.21 (m, 2H), 6.89-6.99 (m, 2H), 6.17 (d, J=3.77 Hz, 1H), 3.90 (s, 3H), 3.80 (br. s., 2H). MS (ESI) m/z [M+H]+calcd: 206.1; found: 206.2.

5-(3-Methoxyphenyl)thiophen-2-amine (126) was prepared from 108 (0.15 g, 0.65 mmol) following the general procedure B as yellow solid (0.06 g, 43%). ¹H NMR (300 MHz, CDC13) δ 7.21 (d, J=7.91 Hz, 1H), 7.05 (d, J=7.72 Hz, 1H), 6.99 (t, J=1.98 Hz, 1H), 6.92 (d, J=3.77 Hz, 1H), 6.71-6.76 (m, 1H), 6.15 (d, J=3.77 Hz, 1 H), 3.80 (s, 3H). MS (ESI) m/z [M+H]+calcd: 206.1; found: 206.2.

5-(4-Methoxyphenyl)thiophen-2-amine (127) was prepared from 109 (0.19 g, 0.80 mmol) following the general procedure B as white solid (0.02 g, 12%). ¹H NMR (300 MHz, CDC13) δ 7.38 (d, J=8.67 Hz, 2H), 6.87 (d, J=8.67 Hz, 2H), 6.79 (d, J=3.77 Hz, 1H), 6.15 (d, J=3.58 Hz, 1H), 3.81 (s, 3H). MS (ESI) m/z [M+H]+calcd: 206.1; found: 206.2.

5-(3-Methylphenyl)thiophen-2-amine (128) was prepared from 110 (0.08 g, 0.35 mmol) following the general procedure B as orange liquid (0.02 g, 35%). ¹H NMR (300 MHz, CHC13) δ 7.24-7.29 (m, J=5.70 Hz, 2H), 7.21 (d, J=7.35 Hz, 1H), 7.00 (d, J=7.35 Hz, 1H), 6.91 (d, J=3.58 Hz, 1H), 6.15 (d, J=3.77 Hz, 1 H), 3.80 (br. s., 2H), 2.35 (s, 3H). MS (ESI) m/z[M+H]+calcd: 190.1; found: 190.3.

5-[3-(Dimethylamino)phenyl]thiophen-2-amine (129) was prepared from 111 (0.06 g, 0.26 mmol) following the general procedure B as orange liquid (0.06 g, quant.). ¹H NMR (300 MHz, CDC13) δ 7.18 (t, J=8.01 Hz, 1H), 6.90 (d, J=3.77 Hz, 1H), 6.84 (d, J=7.72 Hz, 1 H), 6.80 (t, J=1.98 Hz, 1 H), 6.59 (dd, J=2.17, 8.38 Hz, 1H), 6.13 (d, J=3.77 Hz, 1H), 3.77 (br. s., 2H), 2.95 (s, 6H). MS (ESI) m/z [M+H]⁻ calcd: 219.1; found: 219.3.

5-(Pyridin-3-yl)thiophen-2-amine (130) was prepared from 112 (0.07 g, 0.34 mmol) following the general procedure B as white liquid (0.06 g, 14%). ¹H NMR (300 MHz, CDC13) δ 8.73 (d, J=2.07 Hz, 1H), 8.40 (dd, J=1.32, 4.71 Hz, 1 H), 7.70 (td, J=1.88, 8.10 Hz, 1 H), 7.20-7.25 (m, 1 H), 6.99 (d, J=3.77 Hz, 1H), 6.19 (d, J=3.77 Hz, 1H), 3.94 (br. s., 2H). MS (ESI) m/z [M+H]+calcd: 177.1; found: 177.4.

5-(Pyridin-4-yl)thiophen-2-amine (131) was prepared from 113 (0.04 g, 0.17 mmol) following the general procedure B as white liquid (0.005 g, 16%). ¹H

NMR (300 MHz, CDC13) δ 8.44-8.50 (m, 2H), 7.27-7.31 (m, 2H), 7.17 (d, J =3.77 Hz, 1H), 6.18 (d, J=3.77 Hz, 1H), 4.06 (br. s., 2H). MS (ESI) m/z [M+H]+calcd: 177.1; found: 177.3.

5-Phenyl-1,3-thiazol-2-amine hydrobromide (93). To a solution of phenylacetaldehyde (0.49 ml, 4.16 mmol) in dichloromethane (1.5 ml) was added dropwise 15 ml solution of bromine (0.21 ml) at -10° C. The reaction mixture was warmed to room temperature and then refluxed for 16 h. After cooling to room temperature, the reaction mixture was quenched with a saturated solution of sodium bicarbonate and extracted with dichloromethane (3x). The combined organic layers were dried over anhydrous MgSO4, filtered and concentrated to afford crude 2-bromo-2-phenylacetaldehyde which was added to a suspension of thiourea (0.38 g, 5 mmol) in ethanol (10 ml). The reaction mixture was refluxed for 8 h. After cooling to room temperature, solvent was evaporated in vacuo and the residue was purified by column chromatography (silica gel, MeOH/dichloromethane) to provide the product as white solid (0.56 g, 77%). ¹H NMR (300 MHz, CDC13) δ 8.75 (br. s., 2H), 7.36-7.46 (m, 5H), 7.21 (s, 1 H). MS (ESI) m/z [M+H]+calcd: 177.1; found: 177.4. tert-Butyl N-[(3R)-1-phenylpiperidin-3-yl]carbamate (94) To a solution of (R)-3-(Boc-amino)piperidine (0.16 g, 1 mmol) in dichloromethane (4 ml) in a sealed tube was added triethylamine (0.28 ml, 2 mmol), copper acetate (0.20 g, 1.1 mmol), and phenylboronic acid (0.27 g, 2.2 mmol). The reaction was purged with nitrogen, sealed, and heated at 60° C. for 3 days. After cooling to room temperature, the reaction mixture was filtered through Celite, washed with 10%v/v MeOH/DCM. The filtrate was concentrated in vacuo, and the residue was purified by column chromatography (silica gel, MeOH/DCM) to yield the product as colorless liquid (0.09 g, 32%). ¹H NMR (300 MHz, CDC13) δ 7.27-7.29 (m, 1H), 7.22-7.25 (m, 1H), 6.94 (d, J=7.72 Hz, 2H), 6.82-6.89 (m, 1 H), 4.93 (br. s., 1 H), 3.87 (br. s., 1 H), 3.32 (d, J=11.11 Hz, 1 H), 2.94-3.21 (m, 3H), 1.54-1.88 (m, 4H), 1.46 (s, 9H).

(3R)-1-Phenylpiperidin-3-amine hydrochloride (95) A solution of 4 N HCI in 1,4-dioxane was added to 94 (0.09 g, 0.32 mmol) and stirred at room temperature for 1 h. Then the reaction mixture was concentrated under reduced pressure to yield the desired product as white solid (0.07 g, quant.). MS (ESI) m/z [M+H]+calcd: 176.1; found: 176.3.

General procedure C. To a solution of aryl amine (1 eq) in anhydrous chloroform (0.04 M) was added 4-chlorophenyl isocyanate (1 eq) at room temperature. The reaction mixture was then heated at 60° C. for 16 h. The precipitated product was filtered and thoroughly washed with dichloromethane.

General procedure D. To a solution of 4-chloroaniline (1 eq) in toluene (0.2 M) was added acid (1.5 eq) , triethylamine (3 eq), and diphenylphosphoryl azide (1.2 eq). The reaction mixture was heated to 100° C. for 5 min by microwave irradiation. Upon cooling to room temperature, the reaction mixture was diluted with ethyl acetate, acidified to pH 4-5 with 1 N HCI solution. The phases were separated, and the aqueous phase was extracted with ethyl acetate twice. The combined organic fractions were dried over anhydrous magnesium sulphate, filtered, and concentrated in vacuo. The residue was purified by column chromatography (SiO2, ethyl acetate:hexanes) to yield the desired product.

3-(4-Chlorophenyl)-1-12-methoxy-5-[6-(pyrrolidin-1-yl)pyridin-2-yl]phenyl)urea (6) was prepared from 86 (0.02 g, 0.11 mmol) following the general procedure C as white solid (0.03 g, 64%). ¹H NMR (300 MHz, DMSO-d6) δ 9.58 (s, 1 H), 8.85 (d, J=2.07 Hz, 1 H), 8.38 (s, 1 H), 7.75 (dd, J=2.07, 8.48 Hz, 1H), 7.52-7.66 (m, 3H), 7.40 (d, J=8.85 Hz, 2H), 7.15 (d, J=8.67 Hz, 1 H), 7.03 (d, J=7.54 Hz, 1 H), 6.42 (d, J=8.29 Hz, 1 H), 3.99 (s, 3H), 3.48-3.59 (m, 4H), 1.99-2.10 (m, 4H). ¹³C NMR (75 MHz, DMSO-d6) d 156.6, 154.3, 152.3, 148.3, 138.8, 137.7, 132.2, 128.6, 128.3, 125.2, 120.4, 119.5, 116.9, 110.6, 106.6, 104.4, 55.9, 46.2, 25.0. MS (ESI) m/z [M+H]+calcd: 423.1; found: 423.3.

1-(4-Chlorophenyl)-3-(4-phenylpyridin-2-yl)urea (7) was prepared from 87 (0.09 g, 0.51 mmol) following the general procedure C as white solid (0.10 g, 60%). ¹H NMR (300 MHz, DMSO-d6) δ 10.61 (br. s., 1H), 9.57 (s, 1H), 8.36 (d, J=5.27 Hz, 1 H), 7.82 (s, 1 H), 7.73 (d, J=6.78 Hz, 2H), 7.57-7.61 (m, 2H), 7.54 (d, J=7.72 Hz, 2H), 7.49-7.52 (m, 1H), 7.37 (d, J=8.48 Hz, 2H), 7.33-7.35 (m, 1H). ¹³C NMR (75 MHz, DMSO-d6) δ 153.4, 152.1, 149.6, 147.6, 138.0, 137.4, 129.4, 129.2, 128.7, 126.7, 126.0, 120.3, 115.6, 109.0.

MS (ESI) m/z [M+H]+calcd: 324.1; found: 324.2.

1-(4-Chlorophenyl)-3-(6-phenylpyridin-2-yl)urea (8) was prepared from 88 (0.08 g, 0.45 mmol) following the general procedure C as white solid (0.08 g, 56%). ¹H NMR (300 MHz, DMSO-d6) δ 10.73 (br. s., 1H), 9.64 (s, 1H), 8.01 (d, J=7.35 Hz, 2H), 7.83-7.90 (m, 1 H), 7.50-7.60 (m, 5H), 7.47 (d, J=7.91

Hz, 2H), 7.39 (d, J=8.67 Hz, 2H). ¹³C NMR (75 MHz, DMSO-d6) δ 154.0, 152.5, 152.1, 139.6, 138.3, 138.0, 129.2, 128.9, 128.8, 126.5, 126.0, 120.2, 114.3, 110.7. MS (ESI) m/z [M+H]+calcd: 324.1; found: 324.2.

1-(4-Chlorophenyl)-3-(5-phenylpyridin-3-yl)urea (9) was prepared from 89 (0.07 g, 0.37 mmol) following the general procedure C as white solid (0.04 g, 35%). ¹H NMR (300 MHz, DMSO-d6) δ 9.05 (s, 1H), 9.00 (s, 1H), 8.59 (d, J=2.07 Hz, 1 H), 8.51 (d, J=1.51 Hz, 1 H), 8.23 (s, 1 H), 7.69 (d, J=7.16 Hz, 2H), 7.48-7.57 (m, 4H), 7.45 (d, J=6.97 Hz, 1H), 7.35 (d, J=8.85 Hz, 2H). ¹³C NMR (75 MHz, DMSO-d6) δ 152.6, 141.1, 139.1, 138.4, 137.1, 136.4, 135.5, 129.1, 128.6, 128.2, 126.8, 125.7, 123.2, 120.0. MS (ESI) m/z [M+H]+calcd:

324.1; found: 324.1.

1-(4-Chlorophenyl)-3-(2-phenylpyridin-4-yl)urea (10) was prepared from 90 (0.05 g, 0.27 mmol) following the general procedure C as white solid (0.04 g, 45%). ¹H NMR (300 MHz, DMSO-d6) δ 9.26 (s, 1 H), 9.13 (s, 1 H), 8.47 (d, J =5.65 Hz, 1 H), 7.96-8.02 (m, 3H), 7.43-7.55 (m, 5H), 7.33-7.41 (m, 3H).

¹³C NMR (75 MHz, DMSO-d6) δ 156.8, 152.1, 150.1, 147.4, 139.0, 138.1, 128.9, 128.7, 128.7, 126.4, 126.0, 120.2, 111.3, 108.6. MS (ESI) m/z [M+H]+calcd: 324.1; found: 324.2.

1-(4-Chlorophenyl)-3-(5-phenylthiophen-2-yl)urea (11) was prepared from 141 (0.05 g, 0.30 mmol) following the general procedure C as white solid (0.06 g, 57%). ¹H NMR (300 MHz, DMSO-d6) δ 9.83 (s, 1H), 8.96 (s, 1H), 7.53 (dd, J=8.19, 14.22 Hz, 4H), 7.31-7.41 (m, 4H), 7.20-7.26 (m, 2H), 6.58 (d, J=3.96 Hz, 1H). ¹³C NMR (75 MHz, DMSO-d6) δ 151.4, 140.6, 138.3, 134.5, 132.5, 129.0, 128.6, 126.4, 125.7, 124.3, 120.8, 120.0, 110.6. MS (ESI) m/z [M-H]⁻ calcd: 327.1; found: 327.3.

1-(4-Chlorophenyl)-3-(4-phenylthiophen-2-yl)urea (12) was prepared from 91 (0.03 g, 0.16 mmol) following the general procedure D as white solid (0.04 g, 65%). ¹H NMR (300 MHz, DMSO-d6) δ 9.77 (s, 1 H), 9.02 (s, 1 H), 7.65 (d, J =7.54 Hz, 2H), 7.52 (d, J=8.48 Hz, 2H), 7.31-7.45 (m, 4H), 7.28 (d, J=7.35 Hz, 2H), 6.97 (s, 1H). ¹³C NMR (75 MHz, DMSO-d6) δ 151.6, 141.5, 138.3, 137.8, 135.4, 128.7, 128.6, 126.9, 125.6, 119.9, 111.4, 108.2. MS (ESI) m/z [M-H]⁻ calcd: 327.1; found: 327.4.

1-(4-Chlorophenyl)-3-(5-phenylthiophen-3-yl)urea (13) was prepared from 92 (0.03 g, 0.5 mmol) following the general procedure D as white solid (0.04 g, 65%). ¹H NMR (300 MHz, DMSO-d6) δ 8.97 (s, 1 H), 8.87 (s, 1 H), 7.64 (d, J =1.32 Hz, 1 H), 7.61 (s, 1 H), 7.48-7.52 (m, 2H), 7.39-7.46 (m, 3H), 7.27-7.37 (m, 4H). ¹³C NMR (75 MHz, DMSO-d6) δ 152.2, 141.3, 138.7, 137.7, 133.6, 129.1, 128.6, 127.7, 125.3, 125.0, 119.7, 117.6, 106.0. MS (ESI) m/z

[M+H]+calcd: 329.1; found: 329.2. 1-(4-Chlorophenyl)-3-(5-phenyl-1,3-thiazol-2-yl)urea (14) was prepared from 93 (0.03 g, 0.2 mmol) following the general procedure C as white solid (0.02 g, 34%). ¹H NMR (300 MHz, DMSO-d6) δ 10.77 (br. s., 1 H), 9.14 (s, 1H), 7.80 (s, 1H), 7.52-7.62 (m, 4H), 7.48 (d, J=8.85 Hz, 1H), 7.33-7.45 (m, 5H), 7.26-7.32 (m, 1 H). ¹³0 NMR (75 MHz, DMSO-d6) δ 138.5, 137.7, 131.6, 129.1, 128.7, 128.6, 127.3, 126.3, 125.5, 125.4, 120.2, 119.8. MS (ESI) m/z [M+H]+calcd: 330.1; found: 330.0. trans1-(4-Chlorophenyl)-3-(2-phenylcyclopropyl)urea (15) was prepared from trans-2-phenylcyclopropane-1-carboxylic acid (0.06 g, 0.38 mmol) following the general procedure D as white solid (0.05 g, 80%). ¹H NMR (300 MHz, DMSO-d6) δ 8.53 (s, 1 H), 7.44 (s, 1 H), 7.41 (s, 1 H), 7.23-7.31 (m, 4H), 7.17 (d, J=7.16 Hz, 1H), 7.10-7.15 (m, 2H), 6.64 (d, J=2.64 Hz, 1H), 2.72 (dd, J=4.33, 7.16 Hz, 1H), 1.97 (ddd, J=3.30, 6.36, 9.18 Hz, 1H), 1.10-1.21 (m, 2H). ¹³C NMR (75 MHz, DMSO-d6) δ 155.6, 141.4, 139.3, 128.4, 128.1, 125.9, 125.5, 124.6, 119.3, 32.7, 24.5, 15.7. MS (ESI) m/z [M-H]⁻calcd: 285.1; found: 285.6.

cis-1-(4-Chlorophenyl)-3-(2-phenylcyclopropyl)urea (16) was prepared from cis-2-phenylcyclopropane-1-carboxylic acid (0.06 g, 0.38 mmol) following the general procedure D as white solid (0.06 g, 83%). ¹H NMR (300 MHz, CDC13) δ 7.26 (s, 2H), 7.17-7.25 (m, 3H), 7.11-7.16 (m, 2H), 7.00-7.06 (m, 2H), 6.78 (s, 1H), 4.67 (br. s., 1H), 2.84-2.95 (m, 1H), 2.25-2.37 (m, 1H), 1.38 (td, J=6.50, 9.23 Hz, 1H), 1.08 (dt, J=4.33, 6.40 Hz, 1H). ¹³C

NMR (75 MHz, CDC13) δ 155.1, 135.9, 134.6, 134.6, 127.8, 127.4, 127.3, 125.7, 120.3, 27.8, 21.5, 11.8. MS (ESI) m/z [M+H]+calcd: 287.1; found: 287.2.

3-(4-Chlorophenyl)-1-[(3R)-1-phenylpiperidin-3-yl]urea (17) was prepared from 95 (0.05 g, 0.32 mmol) following the general procedure C as white solid (0.03 g, 32%). ¹H NMR (300 MHz, DMSO-d6) δ 8.62 (s, 1H), 7.37-7.44 (m, 2H), 7.23-7.29 (m, 2H), 7.17-7.22 (m, 2H), 6.95 (d, J=7.91 Hz, 2H), 6.76 (t, J=7.16 Hz, 1H), 6.35 (d, J=7.72 Hz, 1 H), 3.77 (dd, J=3.67, 7.82 Hz, 1H), 3.41-3.49 (m, 1H), 3.25 (br. s., 1H), 2.94-3.06 (m, 1H), 2.84 (dd, J=7.82, 11.96 Hz, 1H), 1.70-1.86 (m, 2H), 1.57-1.67 (m, 1H), 1.41-1.53 (m, 1H).

¹³C NMR (75 MHz, DMSO-d6) δ 154.4, 151.2, 139.4, 128.9, 128.4, 124.4, 119.0, 118.7, 116.0, 54.4, 48.9, 45.1, 29.7, 22.6. MS (ESI) m/z [M+H]⁻ calcd: 329.1; found: 329.2.

1-(4-Chlorophenyl)-3-[5-(4-fluorophenyl)thiophen-2-yl]urea (18) was prepared from 114 (0.01 g, 0.04 mmol) following the general procedure C as white solid (0.01 g, 71%). ¹H NMR (300 MHz, DMSO-d6) δ 9.84 (s, 1H), 8.98 (s, 1 H), 7.58 (dd, J=5.46, 8.85 Hz, 2H), 7.50 (d, J=8.85 Hz, 2H), 7.34 (d, J =8.85 Hz, 2H), 7.15-7.25 (m, 3H), 6.57 (d, J=3.96 Hz, 1H). ¹³C NMR (75 MHz, DMSO-d6) δ 151.4, 140.6, 138.3, 131.4, 128.6, 126.3, 126.1, 125.7, 120.9, 120.0, 116.0, 115.7, 110.6. MS (ESI) m/z [M-H]⁻calcd: 345.1; found:

345.1.

1-(4-Chlorophenyl)-3-[5-(3-fluorophenyl)thiophen-2-yl]urea (19) was prepared from 115 (0.07 g, 0.37 mmol) following the general procedure C as white solid (0. g, %). ¹H NMR (300 MHz, DMSO-d6) δ 9.91 (br. s., 1H), 8.99 (br. s., 1H), 7.51 (d, J=7.91 Hz, 2H), 7.35 (d, J=8.85 Hz, 6H), 6.95-7.12 (m, 1H), 6.49-6.69 (m, 1H). ¹³C NMR (75 MHz, DMSO-d6) δ 164.3, 161.1, 151.4, 141.4, 138.2, 136.9, 130.9, 128.6, 125.8, 122.2, 120.3, 120.3, 120.0, 112.8, 110.7. MS (ESI) m/z [M-H]⁻ calcd: 345.1; found: 345.2.

1-(4-Chlorophenyl)-3-[5-(2,4-difluorophenyl)thiophen-2-yl]urea (20) was prepared from 116 (0.03 g, 0.14 mmol) following the general procedure C as white solid (0.04 g, 72%). ¹H NMR (300 MHz, DMSO-d6) δ 9.92 (s, 9H), 9.01 (s, 9H), 7.66-7.78 (m, 9H), 7.51 (d, J=8.85 Hz, 18H), 7.21-7.41 (m, 37H), 7.07-7.18 (m, 9H), 6.62 (d, J=3.96 Hz, 9H). ¹³C NMR (75 MHz, DMSO-d6) δ 151.4, 141.9, 138.2, 128.7, 128.6, 125.8, 124.6, 123.7, 120.0, 118.8, 112.3, 112.0, 109.9, 104.6, 96.3. MS (ESI) m/z [M-F1]⁻ calcd: 363.1; found: 363.3.

1-(4-Chlorophenyl)-3-[5-(2-chlorophenyl)thiophen-2-yl]urea (21) was prepared from 117 (0.03 g, 0.18 mmol) following the general procedure C as white solid (0.01 g, 13%). ¹H NMR (300 MHz, DMSO-d6) δ 9.90 (s, 1H), 8.99 (s, 1H), 7.59 (dd, J=1.51, 7.72 Hz, 1H), 7.48-7.55 (m, 3H), 7.25-7.41 (m, 4H), 7.21 (d, J=3.96 Hz, 1H), 6.62 (d, J=3.96 Hz, 1H). ¹³C NMR (75 MHz, DMSO-d6) δ 151.4, 142.3, 138.2, 132.9, 130.5, 130.5, 130.3, 128.6, 128.5, 128.2, 127.6, 125.8, 125.3, 120.0, 109.7. MS (ESI) m/z calcd: 361.1; found: 361.0.

1-(4-Chlorophenyl)-3-[5-(3-chlorophenyl)thiophen-2-yl]urea (22) was prepared from 118 (0.02 g, 0.08 mmol) following the general procedure C as white solid (0.01 g, 64%). ¹H NMR (300 MHz, DMSO-d6) δ 9.94 (br. s., 1H), 9.02 (br. s., 1H), 7.61 (d, J=1.70 Hz, 1 H), 7.47-7.57 (m, 3H), 7.30-7.43 (m, 4H), 7.21-7.29 (m, 1 H), 6.59 (d, J=3.96 Hz, 1 H). ¹³0 NMR (75 MHz, DMSO-d6) δ 150.4, 140.5, 137.2, 135.6, 132.8, 129.8, 129.5, 127.6, 124.9, 124.8, 122.6, 121.8, 121.3, 119.0, 109.6. MS (ESI) m/z [M-H]⁻ calcd: 361.1; found: 361.3.

1-(4-Chlorophenyl)-3-[5-(4-chlorophenyl)thiophen-2-yl]urea (23) was prepared from 119 (0.02 g, 0.12 mmol) following the general procedure C as white solid (0.03 g, 60%). ¹H NMR (300 MHz, DMSO-d6) δ 9.88 (s, 1H), 8.98 (s, 1H), 7.54 (dd, J=8.57, 18.37 Hz, 4H), 7.37 (dd, J=8.57, 17.80 Hz, 4H), 7.26 (d, J=3.77 Hz, 1 H), 6.58 (d, J=3.77 Hz, 1 H). ¹³0 NMR (75 MHz, DMSO-d6) δ 150.4, 140.1, 137.2, 132.4, 130.0, 129.6, 127.9, 127.6, 124.8, 120.6, 119.0, 109.6. MS (ESI) m/z [M-H]⁻ calcd: 361.1; found: 361.2. 1-(4-Chlorophenyl)-3-[5-(3,4-dichlorophenyl)thiophen-2-yl]urea (24) was prepared from 120 (0.01 g, 0.03 mmol) following the general procedure C as white solid (0.01 g, 67%). ¹H NMR (300 MHz, DMSO-d6) δ 9.97 (s, 1H), 9.02 (s, 1H), 7.82 (d, J=2.07 Hz, 1H), 7.57-7.62 (m, 1H), 7.48-7.54 (m, 3H), 7.39 (d, J=3.96 Hz, 1 H), 7.35 (d, J=8.85 Hz, 2H), 6.60 (d, J=3.96 Hz, 1 H). ¹³C NMR (75 MHz, DMSO-d6) δ 151.4, 142.0, 138.2, 135.3, 131.7, 131.0, 129.4, 128.6, 128.2, 125.8, 125.5, 124.2, 122.9, 120.0, 110.6. MS (ESI) m/z [M-H]⁻ calcd: 397.1; found: 397.1.

1-(4-Chlorophenyl)-3-[5-(3,5-dichlorophenyl)thiophen-2-yl]urea (25) was prepared from 121 (0.02 g, 0.06 mmol) following the general procedure C as white solid (0.01 g, 54%). ¹H NMR (300 MHz, DMSO-d6) δ 10.01 (br. s., 1H), 9.04 (br. s., 1 H), 7.55-7.62 (m, 2H), 7.44-7.54 (m, 3H), 7.30-7.42 (m, 3H), 6.61 (s., 1H). ¹³C NMR (75 MHz, DMSO-d6) δ 150.3, 141.4, 137.0, 137.0, 133.6, 127.7, 127.6, 124.8, 124.1, 122.6, 121.3, 119.0, 109.6. MS (ESI) m/z [M-H]⁻ calcd: 397.1; found: 397.2. 3-[5-(3-Acetylphenyl)thiophen-2-yl]-1-(4-chlorophenyl)urea (26) was prepared from 122 (0.06 g, 0.28 mmol) following the general procedure C as yellow solid (0.01 g, 7%). ¹H NMR (300 MHz, DMSO-d6) δ 9.91 (br. s., 1H), 9.01 (br. s., 1H), 8.05 (s, 1H), 7.77-7.88 (m, 2H), 7.51 (d, J=5.27 Hz, 3H), 7.30-7.42 (m, 3H), 6.56-6.65 (m, 1H), 2.63 (s, 3H). ¹³C NMR (75 MHz, DMSO-d6) δ 197.8, 151.4, 141.3, 138.2, 137.5, 134.9, 131.3, 129.4, 128.7, 128.6, 126.0, 125.8, 123.4, 121.9, 120.0, 110.6, 26.8. MS (ESI) m/z [M-H]⁻ calcd: 369.1; found: 369.1.

Methyl 3-(5-{[(4-chlorophenyl)carbamoyl]aminol}thiophen-2-yl)benzoate (27) was prepared from 123 (0.04 g, 0.16 mmol) following the general procedure C as white solid (0.05 g, 85%). ¹H NMR (300 MHz, DMSO-d6) δ 9.93 (s, 1H), 9.01 (s, 1H), 8.07 (s, 1H), 7.86 (d, J=7.91 Hz, 1H), 7.78 (d, J=7.72 Hz, 1H), 7.46-7.57 (m, 3H), 7.34 (d, J=8.85 Hz, 3H), 6.60 (d, J=3.77 Hz, 1H), 3.88 (s, 3H). ¹³C NMR (75 MHz, DMSO-d6) δ 166.0, 151.4, 141.3, 138.2, 135.0, 131.0, 130.4, 129.5, 128.7, 128.6, 126.7, 125.8, 124.4, 121.8, 120.0, 110.6, 52.2. MS (ESI) m/z [M-H]⁻ calcd: 385.1; found: 385.4.

1-(4-Chlorophenyl)-3-[5-(3-methanesulfonylphenyl)thiophen-2-yl]urea (28) was prepared from 124 (0.04 g, 0.15 mmol) following the general procedure C as white solid (0.05 g, 75%). ¹H NMR (300 MHz, DMSO-d6) δ 9.98 (br. s., 1H), 9.03 (br. s., 1H), 8.03 (s, 1H), 7.90 (s, 1H), 7.70-7.79 (m, 1 H), 7.59-7.68 (m, 1 H), 7.48-7.57 (m, 2H), 7.40-7.46 (m, 1 H), 7.28-7.39 (m, 2H), 6.63 (s, 1H), 3.29 (s, 3H). ¹³C NMR (75 MHz, DMSO-d6) δ 151.4, 142.0, 141.7, 138.2, 135.7, 130.2, 130.1, 128.8, 128.6, 125.9, 124.2, 122.8, 122.0, 120.0, 110.6, 43.4. MS (ESI) m/z [M-H]⁻ calcd: 405.1; found: 405.4. 1-(4-Chlorophenyl)-3-[5-(2-methoxyphenyl)thiophen-2-yl]urea (29) was prepared from 125 (0.02 g, 0.15 mmol) following the general procedure C as white solid (0.04 g, 78%). ¹H NMR (300 MHz, DMSO-d6) δ 9.72 (br. s., 1H), 8.93 (br. s., 1 H), 7.58-7.68 (m, 1 H), 7.46-7.57 (m, 2H), 7.26-7.39 (m, 3H), 7.15-7.24 (m, 1H), 7.04-7.13 (m, 1H), 6.92-7.02 (m, 1H), 6.53-6.63 (m, 1 H), 3.89 (s, 3H). ¹³0 NMR (75 MHz, DMSO-d6) δ 154.7, 151.4, 141.4, 138.4, 128.6, 128.5, 127.3, 126.8, 125.6, 123.1, 122.7, 120.9, 119.9, 112.1, 109.7, 55.6. MS (ESI) m/z [M-H]⁻ calcd: 357.1; found: 357.3.

1-(4-Chlorophenyl)-3-[5-(3-methoxyphenyl)thiophen-2-yl]urea (30) was prepared from 126 (0.04 g, 0.28 mmol) following the general procedure C as white solid (0.06 g, 55%). ¹H NMR (300 MHz, DMSO-d6) δ 9.84 (s, 1H), 8.98 (s, 1H), 7.52 (d, J=8.67 Hz, 2H), 7.35 (d, J=8.85 Hz, 2H), 7.21-7.28 (m, 2H), 7.06-7.17 (m, 2H), 6.75-6.85 (m, 1H), 6.58 (d, J=3.77 Hz, 1H), 3.80 (s, 3H). ¹³C NMR (75 MHz, DMSO-d6) δ 159.7, 151.4, 140.7, 138.3, 135.8, 132.4, 130.0, 128.6, 125.8, 121.2, 120.0, 116.8, 112.1, 110.5, 109.7, 55.0. MS (ESI) m/z [M-H]⁻ calcd: 357.1; found: 357.2.

1-(4-Chlorophenyl)-3-[5-(4-methoxyphenyl)thiophen-2-yl]urea (31) was prepared from 127 (0.02 g, 0.10 mmol) following the general procedure C as white solid (0.03 g, 86%). ¹H NMR (300 MHz, DMSO-d6) δ 9.73 (br. s., 1H), 8.93 (br. s., 1 H), 7.49 (t, J=8.95 Hz, 4H), 7.34 (d, J=8.48 Hz, 2H), 7.07 (d, J=3.01 Hz, 1H), 6.94 (d, J=8.29 Hz, 2H), 6.54 (d, J=3.01 Hz, 1H), 3.76 (s, 3H). ¹³0 NMR (75 MHz, DMSO-d6) 6 158.6, 151.9, 140.0, 138.8, 133.3, 129.1, 127.7, 126.2, 126.1, 120.4, 119.9, 114.9, 111.1, 55.6. MS (ESI) m/z [M-H]⁻ calcd: 357.1; found: 357.3.

1-(4-Chlorophenyl)-3-[5-(3-methylphenyl)thiophen-2-yl]urea (32) was prepared from 128 (0.02 g, 0.12 mmol) following the general procedure C as white solid (0.03 g, 62%). ¹H NMR (300 MHz, DMSO-d6) δ 9.81 (br. s., 1H), 8.96 (br. s., 1 H), 7.46-7.57 (m, 2H), 7.30-7.43 (m, 4H), 7.17-7.29 (m, 2H), 6.98-7.07 (m, 1H), 6.51-6.63 (m, 1H), 2.33 (s, 3H). ¹³0 NMR (75 MHz, DMSO-d6) δ 151.4, 140.4, 138.3, 138.1, 134.4, 132.7, 128.8, 128.6, 127.1, 125.7, 124.9, 121.5, 120.7, 119.9, 110.6, 21.0. MS (ESI) m/z [M-H]⁻ calcd: 341.1; found: 341.4.

1-(4-Chlorophenyl)-3-{5-[3-(dimethylamino)phenyl]thiophen-2-yl}urea

(33) was prepared from 129 (0.06 g, 0.26 mmol) following the general procedure C as white solid (0.06 g, 58%). ¹H NMR (300 MHz, DMSO-d6) δ 9.79 (br. s., 1H), 8.97 (br. s., 1H), 7.47-7.58 (m, 2H), 7.35 (d, J=6.59 Hz, 2H), 7.10-7.22 (m, 2H), 6.79-6.92 (m, 2H), 6.51-6.65 (m, 2H), 2.93 (s, 6H). ¹³C NMR (75 MHz, DMSO-d6) δ 151.4, 150.7, 140.1, 138.3, 135.0, 133.7, 129.4, 128.6, 125.7, 120.4, 119.9, 112.8, 110.9, 110.4, 108.1. MS (ESI) m/z [M-H]⁻ calcd: 370.1; found: 370.2.

1-(4-Chlorophenyl)-3-[5-(pyridin-3-yl)thiophen-2-yl]urea (34) was prepared from 130 (0.08 g, 0.05 mmol) following the general procedure C as white solid (0.02 g, 88%). ¹H NMR (300 MHz, DMSO-d6) δ 9.94 (s, 1H), 9.01 (s, 1H), 8.81 (d, J=2.07 Hz, 1H), 8.40 (dd, J=1.32, 4.71 Hz, 1H), 7.93 (td, J =1.81, 8.05 Hz, 1H), 7.51 (d, J=9.04 Hz, 2H), 7.33-7.41 (m, 4H), 6.62 (d, J =3.96 Hz, 1H). ¹³0 NMR (75 MHz, DMSO-d6) δ 151.4, 147.2, 145.2, 141.7, 138.2, 131.3, 130.5, 128.6, 125.8, 123.9, 122.4, 120.0, 119.8, 110.6. MS (ESI) m/z [M-H]⁻ calcd: 328.1; found: 328.4.

1-(4-Chlorophenyl)-3-[5-(pyridin-4-yl)thiophen-2-yl]urea (35) was prepared from 131 (0.004 g, 0.03 mmol) following the general procedure C as yellow solid (0.007 g, 75%). ¹H NMR (300 MHz, DMSO-d6) δ 10.07 (br. s., 1H), 9.05 (br. s., 1H), 8.42-8.53 (m, 2H), 7.43-7.61 (m, 5H), 7.36 (d, J=7.91 Hz, 2H), 6.62-6.68 (m, 1H). ¹³0 NMR (75 MHz, DMSO-d6) δ 150.0, 143.2, 141.4, 138.1, 128.9, 128.6, 125.9, 124.3, 120.1, 119.8, 118.3, 110.7. MS (ESI) m/z [M-H]⁻ calcd: 328.1; found: 328.4.

2-Methyl-2-phenylpropan-1-amine hydrochloride (133) To a solution of 2-methyl-2-phenylpropanenitrile (0.15 ml, 1 mmol) in anhydrous THF (4 ml) was added BH3.Me2S (0.5 ml, 1 mmol) dropwise at 0° C. The reaction mixture was raised to room temperature and then refluxed for 16 h. Upon cooling to room temperature, methanol was added slowly to quench the reaction was the mixture was concentrated in vacuo. This quenching was repeated twice more. The crude product was dissolved in a minimum amount of diethyl ether and treated with 2N ethereal HCI. The white solid precipitate was filtered and washed with ice cold diethyl ether to give the pure product (0.17 g, 92%). ¹H NMR (300 MHz, CD3OD) δ 7.37-7.48 (m, 4H), 7.26-7.32 (m, 1H), 3.18 (s, 2H), 1.44 (s, 6H). MS (ESI) m/z [M+H]⁻ calcd: 150.1; found: 150.2. 2,2-Difluoro-2-phenylethan-1-amine hydrochloride (134) was prepared from 2,2-difluoro-2-phenylacetamide (0.17 g, 1 mmol) following the same procedure for 14014-151 to obtain the desired product as yellow solid (0.11 g, 58%). ¹H NMR (300 MHz, CD3OD) δ 7.39-7.76 (m, 5H), 3.70 (d, J=15.64 Hz, 2H). MS (ESI) m/z [M+H]⁻ calcd: 158.1; found: 158.2. Methyl 2,2-dimethyl-3-phenylpropanoate (138). To a solution of 2M LDA in THF (1.2 ml, 1.2 mmol) at -78 ⁰0 was added methyl isobutyrate (0.11 mmol, 1 mmol) dropwise. After 1 h, a solution of benzyl bromide (0.12 ml, 1.2 ml) in THF (0.5 ml) was added dropwise. The reaction was stirred at -78° C. for 1.5 h and slowly warmed to room temperature. The reaction was then quenched with saturated NH4C1 and extracted with ethyl acetate three times. The combined organic layers were dried over anhydrous MgSO4, filtered, and concentrated in vacuo to afford the crude product was orange liquid (2.0 g, quant.). ¹H NMR (300 MHz, CDC13) δ 7.31-7.35 (m, 1 H), 7.20-7.25 (m, 2H), 7.07-7.13 (m, 2H), 3.66 (s, 3H), 2.85 (s, 2H), 1.18 (s, 6H). 2,2-Dimethyl-3-phenylpropanoic acid (139). To a solution of methyl 2,2-dimethyl-3-phenylpropanoate (2.0 g, 10 mmol) in methanol (20 ml) was added a solution of lithium hydroxide (1.20 g) in water (20 ml) at room temperature. The reaction mixture was stirred for 3 h before the reaction volume was reduced in by evaporation in vacuo. The reaction mixture was then diluted with ethyl acetate and adjusted to pH 3 by 4N HCI. The phases were separated, and the aqueous layer was extracted twice more with ethyl acetate. The combined organic layers were dried over anhydrous MgSO4, filtered, and concentrated in vacuo to afford the crude product as white solid (1.8 g, quant.). ¹H NMR (300 MHz, CDC13) δ 7.23-7.29 (m, 3H), 7.14-7.19 (m, 2H), 2.89 (s, 2H), 1.21 (s, 6H). MS (ESI) m/z [M-H]⁻ calcd: 177.1; found: 177.2. trans-2-Phenylcyclopropane-1-carboxamide (132). To a solution of trans-2-phenylcyclopropane-1-carboxylic acid (0.16 g, 1 mmol) in anhydrous dichloromethane (5 ml) was added oxalyl chloride (0.1 ml, 1.21 mmol) and 2-3 drops of DMF. The reaction was stirred at room temperature for 3 h before the solvent was evaporated in vacuo. The residue was then diluted in anhydrous acetonitrile (5 ml) and treated with concentrated aqueous ammonium hydroxide 25% (0.5 ml). The reaction was stirred at room temperature for 16 h before being diluted with ethyl acetate. The phases were separated, and the aqueous layer was extracted twice with ethyl acetate. The combined organic layers were dried over anhydrous MgSO4, filtered, and concentrated in vacuo to yield the product as white solid (0.17 g, quant.). ¹H NMR (300 MHz, CDC13) δ 7.27-7.33 (m, 2H), 7.19-7.24 (m, 1H), 7.08-7.13 (m, 2H), 5.29-5.69 (m, 2H), 2.48-2.56 (m, 1H), 1.60-1.71 (m, 2H), 1.27-1.36 (m, 1H). MS (ESI) m/z [M+H]⁻ calcd: 162.1; found: 162.2. trans-(2-Phenylcyclopropyl)methanamine hydrochloride (135) was prepared from 132 (0.17 g, 1 mmol) following the same procedure for 14014-151 to obtain the desired product as yellow solid (0.14 g, 78%). ¹H NMR (300 MHz, CD3OD) δ 7.06-7.44 (m, 5H), 3.54-3.62 (m, 2H), 2.95-3.05 (m, 1 H), 1.55-1.64 (m, 2H), 1.03-1.14 (m, 1H). MS (ESI) m/z [M+H]+calcd: 148.1; found: 148.2.

2-(2,4,6-Trifluorophenyl)ethan-1-amine (136). To a solution of LiAlH₄ in THF at 0° C. was added anhydrous AlCl₃. After 5 min, 2,4,6-trifluorobenzonitrile (0.13 ml, 1 mmol) was added dropwise slowly. After stirring at room temperature for 1 h, the remaining LiA1H4 was quenched cautiously with water, and then with 1.6 ml of 6N H2504. The pH of the solution was adjusted to 11 with KOH pellets and extracted three times with ethyl acetate. The combined organic layers were dried over anhydrous MgSO4, filtered and concentrated in vacuo. The crude yellow liquid product (0.16 g, 90%) was used for the next step without further purification. ¹H NMR (300 MHz, CDC13) δ 6.59-6.69 (m, 3H), 2.87-2.95 (m, 2H), 2.71-2.80 (m, 2H). MS (ESI) m/z [M+H]+calcd: 176.1; found: 176.5.

2-(2,3,4,5,6-Pentafluorophenyl)ethan-1-amine (137) was prepared from 2,3,4,5,6-pentafluorobenzonitrile (0.12 ml, 1 mmol) following the same procedure for 14014-165 to obtain the desired product as yellow liquid (0.17 g, 81%). ¹H NMR (300 MHz, CDC13) δ 3.53-3.86 (m, 1 H), 2.77-3.08 (m, 1H), 1.57-2.17 (m, 2H). MS (ESI) m/z [M+H]⁻ calcd: 212.1; found: 212.1.

1-Benzyl-3-(4-chlorophenyl)urea (36) was prepared from benzylamine (0.05 g, 0.32 mmol) following the general procedure C as white solid (0.06 g, 71%). ¹H NMR (300 MHz, DMSO-d6) δ 8.71 (s, 1H), 7.44 (d, J=8.85 Hz, 2H), 7.20-7.37 (m, 7H), 6.66 (t, J=5.75 Hz, 1 H), 4.30 (d, J=6.03 Hz, 2H). ¹³C NMR (75 MHz, DMSO-d6) δ 155.0, 140.2, 139.4, 128.4, 128.3, 127.1, 126.7, 124.5, 119.2, 42.7. MS (ESI) m/z [M+H]+calcd: 261.1; found: 261.3.

3-(4-Chlorophenyl)-1-(3-phenylpropyl)urea (37) was prepared from 3-phenylpropylamine (0.05 ml, 0.32 mmol) following the general procedure C as white solid (0.08 g, 82%). ¹H NMR (300 MHz, CDC13) δ 7.17-7.31 (m, 7H), 7.09 (d, J=6.78 Hz, 2H), 5.42 (t, J=5.37 Hz, 1H), 3.18 (q, J=6.78 Hz, 2H), 2.57 (t, J=7.63 Hz, 2H), 1.70-1.78 (m, 2H). ¹³C NMR (75 MHz, CHC13) δ 156.1, 141.3, 137.4, 129.1, 128.5, 128.4, 128.3, 126.0, 121.5, 39.9, 33.1, 31.6. MS (ESI) m/z [M+H]+calcd: 289.1; found: 289.3.

3-(4-Chlorophenyl)-1-(2-phenylethyl)urea (44) was prepared from phenethylamine (0.04 ml, 0.32 mmol) following the general procedure C as white solid (0.06 g, 63%). ¹H NMR (300 MHz, CDC13) δ 7.29-7.35 (m, 2H), 7.15-7.25 (m, 7H), 6.12 (br. s., 1H), 4.58 (br. s., 1H), 3.53 (q, J=6.59 Hz, 2H), 2.85 (t, J=6.69 Hz, 2H). ¹³C NMR (75 MHz, CHC13) δ 155.1, 138.9, 137.0, 129.2, 128.9, 128.8, 128.7, 126.6, 122.0, 41.5, 36.0. MS (ESI) m/z [M+H]+calcd: 275.1; found: 275.2.

1-[2-(4-tert-Butylphenyl)ethyl]-3-(4-chlorophenyl)urea (45) was prepared from 4-tert-butylphenethylamine (0.05 g, 0.32 mmol) following the general procedure C as white solid (0.03 g, 29%). ¹H NMR (300 MHz, DMSO-d6) δ 8.63 (s, 1 H), 7.41 (d, J=8.85 Hz, 2H), 7.33 (d, J=8.29 Hz, 2H), 7.25 (d, J=8.85 Hz, 2H), 7.16 (d, J=8.10 Hz, 2H), 6.15 (t, J=5.46 Hz, 1H), 3.27-3.32 (m, 2H), 2.70 (t, J=7.16 Hz, 2H), 1.27 (s, 9H).¹³C NMR (75 MHz, DMSO-d6) d 154.9, 148.3, 139.5, 136.4, 128.4, 128.3, 125.1, 124.4, 119.0, 35.2, 34.0, 31.2. MS (ESI) m/z [M+H]+calcd: 331.1; found: 331.2.

3-(4-Chlorophenyl)-1-[2-(4-phenylphenyl)ethyl]urea (46) was prepared from 4-phenylphenethylamine (0.05 g, 0.32 mmol) following the general procedure C as white solid (0.09 g, 76%). ¹H NMR (300 MHz, DMSO-d6) 6 8.64 (s, 1H), 7.63 (dd, J=8.10, 10.55 Hz, 4H), 7.31-7.50 (m, 7H), 7.25 (d, J =8.85 Hz, 2H), 6.19 (t, J=5.56 Hz, 1H), 3.36-3.42 (m, 2H), 2.80 (t, J=6.97

Hz, 2H). ¹³C NMR (75 MHz, DMSO-d6) 6 155.0, 140.0, 139.5, 138.8, 138.0, 129.2, 128.9, 128.4, 127.2, 126.6, 126.5, 124.4, 119.0, 35.3, 30.6. MS (ESI) m/z [M+H]+calcd: 351.1; found: 351.2.

3-(4-Chlorophenyl)1-[2-(4-chlorophenyl)ethyl]urea (47) was prepared from 4-chlorophenethylamine (0.05 g, 0.32 mmol) following the general procedure C as white solid (0.07 g, 66%). ¹H NMR (300 MHz, DMSO-d6) 6 8.61 (s, 1H), 7.38 (dd, J=8.57, 11.59 Hz, 4H), 7.22-7.29 (m, 4H), 6.14 (t, J =5.56 Hz, 1H), 3.28-3.33 (m, 2H), 2.74 (t, J=6.97 Hz, 2H). ¹³C NMR (75 MHz, DMSO-d6) 6 154.9, 139.5, 138.5, 130.7, 130.5, 128.4, 128.2, 124.4, 119.0, 35.0. MS (ESI) m/z [M+H]+calcd: 309.1; found: 309.1.

3-(4-Chlorophenyl)1-[2-(4-nitrophenyl)ethyl]urea (48) was prepared from 4-nitrophenethylamine hydrochloride (0.07 g, 0.32 mmol) following the general procedure C as white solid (0.06 g, 54%). ¹H NMR (300 MHz, DMSO-d6) δ 8.64 (s, 1 H), 8.18 (d, J=8.67 Hz, 2H), 7.53 (d, J=8.67 Hz, 2H), 7.40 (d, J=9.04 Hz, 2H), 7.25 (d, J=9.04 Hz, 2H), 6.22 (t, J=5.75 Hz, 1 H), 3.37-3.43 (m, 2H), 2.90 (t, J=6.88 Hz, 2H). ¹³C NMR (75 MHz, DMSO-d6) 6 154.9, 148.0, 146.1, 139.4, 130.0, 128.4, 124.4, 123.4, 119.1, 35.5, 30.6. MS (ESI) m/z [M+H]+calcd: 320.1; found: 320.2.

3-(4-Chlorophenyl)1-[2-(4-hydroxy-3-methoxyphenyl)ethyl]urea (49) was prepared from 4-hydroxy-3-methoxyphenethylamine (0.07 g, 0.32 mmol) following the general procedure C as white solid (0.03 g, 27%). ¹H NMR (300 MHz, DMSO-d6) δ 8.73 (s, 1 H), 8.63 (s, 1 H), 7.41 (d, J=8.85 Hz, 2H), 7.25 (d, J=8.67 Hz, 2H), 6.77 (s, 1H), 6.67-6.72 (m, 1H), 6.58-6.64 (m, 1H), 6.08 (t, J=5.46 Hz, 1H), 3.75 (s, 3H), 3.24-3.30 (m, 2H), 2.63 (t, J=6.97 Hz, 2H). ¹³C NMR (75 MHz, DMSO-d6) 6 154.9, 147.4, 144.8, 139.5, 130.1, 128.4, 124.3, 120.7, 119.0, 115.4, 112.8, 55.5, 35.3. MS (ESI) m/z [M-H]⁻ calcd: 319.1; found: 319.4.

3-(4-Chlorophenyl)-1-{2-[3-(dimethylamino)phenyl]ethyl}urea (50) was prepared from 3-dimethylaminophenethylamine (0.07 g, 0.33 mmol) following the general procedure C as white solid (0.05 g, 50%). ¹H NMR (300 MHz, CDC13) δ 7.13-7.24 (m, 5H), 6.59-6.64 (m, 1 H), 6.53-6.58 (m, 2H), 6.14 (s, 1 H), 4.60-4.67 (m, 1 H), 3.53 (q, J=6.53 Hz, 2H), 2.93 (s, 6H), 2.80 (t, J=6.69 Hz, 2H). ¹³C NMR (75 MHz, CHC13) δ 155.2, 151.0, 139.7, 137.2, 129.5, 129.2, 128.8, 122.0, 116.9, 113.0, 110.9, 41.6, 40.6, 36.4. MS (ESI) m/z [M+H]+calcd: 318.1; found: 318.2.

3-(4-Chlorophenyl)-1-{2-[4-(dimethylamino)phenyl]ethyl}urea (51) was prepared from 4-dimethylaminophenethylamine (0.03 g, 0.18 mmol) following the general procedure C as white solid (0.01 g, 17%). ¹H NMR (300 MHz, DMSO-d6) δ 8.63-8.71 (m, 1H), 7.41 (d, J=9.04 Hz, 2H), 7.24 (d, J=8.85 Hz, 2H), 7.04 (d, J=8.67 Hz, 2H), 6.68 (d, J=8.67 Hz, 2H), 6.08-6.16 (m, 1 H), 3.26 (d, J=6.22 Hz, 2H), 2.85 (s, 6H), 2.61 (t, J=7.16 Hz, 2H). ¹³0 NMR (75 MHz, DMSO-d6) δ 154.9, 149.1, 139.5, 129.0, 128.4, 126.9, 124.3, 119.0, 112.7, 40.9, 40.3, 34.8. MS (ESI) m/z [M+H]+calcd: 318.1; found: 318.2. 3-(4-Chlorophenyl)1-[2-(4-methanesulfonylphenyl)ethyl]urea (52) was prepared from 2-(4-methylsulfonyl-phenyl)ethylamine hydrochloride (0.08 g, 0.32 mmol) following the general procedure C as white solid (0.04 g, 35%). ¹H NMR (300 MHz, DMSO-d6) δ 8.64 (s, 1H), 7.87 (d, J=8.29 Hz, 2H), 7.52 (d,

J=8.29 Hz, 2H), 7.41 (d, J=8.85 Hz, 2H), 7.25 (d, J=8.85 Hz, 2H), 6.21 (t, J=5.56 Hz, 1 H), 3.36-3.43 (m, 2H), 3.20 (s, 3H), 2.87 (t, J=6.88 Hz, 2H). ¹³C NMR (75 MHz, DMSO-d6) δ 155.0, 145.8, 139.4, 138.7, 129.6, 128.4, 127.0, 124.4, 119.1, 43.6, 35.5, 30.6. MS (ESI) m/z [M+H]+calcd: 353.1; found: 353.2.

3-(4-Chlorophenyl)1-[2-(2-methoxyphenyl)ethyl]urea (53) was prepared from 2-methoxyphenethylamine (0.05 ml, 0.32 mmol) following the general procedure C as white solid (0.05 g, 46%). ¹H NMR (300 MHz, DMSO-d6) δ 8.58 (br. s., 1 H), 7.41 (d, J=8.67 Hz, 2H), 7.25 (d, J=8.48 Hz, 2H), 7.11-7.22 (m, 2H), 6.97 (d, J=7.54 Hz, 1H), 6.89 (t, J=7.16 Hz, 1H), 6.12 (br. s., 1H), 3.78 (s, 3H), 3.29 (d, J=5.65 Hz, 2H), 2.68-2.77 (m, 2H). ¹³C NMR (75 MHz, DMSO-d6) δ 157.3, 154.9, 139.5, 130.0, 128.4, 127.5, 127.2, 124.3, 120.2, 119.0, 110.7, 55.3, 30.3. MS (ESI) m/z [M+H]+calcd: 305.1; found: 305.4.

3-(4-Chlorophenyl)1-[2-(3-methoxyphenyl)ethyl]urea (54) was prepared from 3-methoxyphenethylamine (0.05 ml, 0.32 mmol) following the general procedure C as white solid (0.05 g, 47%). ¹H NMR (300 MHz, DMSO-d6) δ 8.63 (s, 1H), 7.41 (d, J=8.85 Hz, 2H), 7.23-7.29 (m, 2H), 7.19-7.23 (m, 1 H), 6.75-6.84 (m, 3H), 6.13 (t, J=5.65 Hz, 1 H), 3.74 (s, 3H), 3.29-3.34 (m, 2H), 2.72 (t, J=7.06 Hz, 2H). ¹³C NMR (75 MHz, DMSO-d6) δ 159.3, 154.9, 141.0, 139.5, 129.3, 128.4, 124.4, 120.9, 119.0, 114.2, 111.5, 54.9, 40.4, 35.7. MS (ESI) m/z [M+H]+calcd: 305.1; found: 305.4.

3-(4-Chlorophenyl)1-[2-(3-methoxyphenyl)ethyl]urea (55) was prepared from 4-methoxyphenethylamine (0.05 ml, 0.32 mmol) following the general procedure C as white solid (0.07 g, 66%). ¹H NMR (300 MHz, DMSO-d6) δ 8.62 (s, 1H), 7.41 (d, J=9.04 Hz, 2H), 7.25 (d, J=8.85 Hz, 2H), 7.15 (d, J=8.48 Hz, 2H), 6.87 (d, J=8.48 Hz, 2H), 6.11 (t, J=5.56 Hz, 1 H), 3.72 (s, 3H), 3.24-3.31 (m, 2H), 2.67 (t, J=7.06 Hz, 2H). ¹³C NMR (75 MHz, DMSO-d6) δ 157.7, 154.9, 139.5, 131.3, 129.6, 128.4, 124.3, 119.0, 113.8, 55.0, 34.8. MS (ESI) m/z [M+H]+calcd: 305.1; found: 305.4.

3-(4-Chlorophenyl)1-[2-(3,4-dimethoxyphenyl)ethyl]urea (56) was prepared from 3,4-dimethoxyphenethylamine (0.05 ml, 0.32 mmol) following the general procedure C as white solid (0.04 g, 38%). ¹H NMR (300 MHz, CDC13) δ 7.16-7.24 (m, 4H), 6.75-6.81 (m, 1 H), 6.67-6.74 (m, 2H), 6.52 (s, 1H), 4.83 (t, J=5.18 Hz, 1H), 3.84 (s, 3H), 3.81 (s, 3H), 3.49 (q, J=6.66 Hz, 2H), 2.77 (t, J=6.69 Hz, 2H). ¹³C NMR (75 MHz, CHC13) δ 155.3, 149.0, 147.7, 137.3, 131.4, 129.2, 128.6, 121.7, 120.7, 112.0, 111.4, 55.9, 55.8, 41.4, 35.6. MS (ESI) m/z [M+H]+calcd: 335.1; found: 335.3.

3-(4-Chlorophenyl)1-[2-(3,5-dimethoxyphenyl)ethyl]urea (57) was prepared from 3,5-dimethoxyphenethylamine (0.06 ml, 0.32 mmol) following the general procedure C as white solid (0.08 g, 70%). ¹H NMR (300 MHz, DMSO-d6) δ 8.64 (s, 1 H), 7.41 (d, J=8.67 Hz, 2H), 7.25 (d, J=8.67 Hz, 2H), 6.40 (s, 2H), 6.35 (br. s., 1H), 6.11 (t, J=5.09 Hz, 1H), 3.72 (s, 6H), 3.31-3.36 (m, 2H), 2.68 (t, J=6.78 Hz, 2H). ¹³C NMR (75 MHz, DMSO-d6) δ 160.4, 154.9, 141.8, 139.5, 128.4, 124.4, 119.0, 106.6, 98.0, 55.0, 36.0. MS (ESI) m/z [M+H]+calcd: 335.1; found: 335.3.

3-(4-Chlorophenyl)1-[2-(4-hydroxyphenyl)ethyl]urea (58) was prepared from 4-hydroxyphenethylamine (0.04 g, 0.32 mmol) following the general procedure C as white solid (0.07 g, 78%). ¹H NMR (300 MHz, DMSO-d6) δ 9.19 (br. s., 1H), 8.62 (s, 1H), 7.41 (d, J=8.67 Hz, 2H), 7.25 (d, J=8.85 Hz, 2H), 7.02 (d, J=8.29 Hz, 2H), 6.69 (d, J=8.29 Hz, 2H), 6.10 (t, J=5.46 Hz, 1H), 3.26 (q, J=6.66 Hz, 2H), 2.62 (t, J=7.16 Hz, 2H). ¹³C NMR (75 MHz, DMSO-d6) δ 155.6, 154.9, 139.5, 129.5, 128.4, 124.3, 119.0, 115.1, 34.9. MS (ESI) m/z calcd: 289.1; found: 289.2.

3-(4-Chlorophenyl)1-[2-(4-methylphenyl)ethyl]urea (59) was prepared from 4-methylphenethylamine (0.05 ml, 0.32 mmol) following the general procedure C as white solid (0.03 g, 32%). ¹H NMR (300 MHz, DMSO-d6) δ 8.62 (s, 1H), 7.41 (d, J=8.85 Hz, 2H), 7.25 (d, J=8.85 Hz, 2H), 7.08-7.15 (m, 4H), 6.11 (t, J=5.46 Hz, 1H), 3.26-3.32 (m, 2H), 2.69 (t, J=7.16 Hz, 2H), 2.27 (s, 3H). ¹³0 NMR (75 MHz, DMSO-d6) δ 154.9, 139.5, 136.3, 135.0, 128.9, 128.5, 128.4, 124.3, 119.0, 35.3, 20.6. MS (ESI) m/z [M+H]+calcd: 289.1; found: 289.2.

3-(4-Chlorophenyl)1-[2-(3-methylphenyl)ethyl]urea (60) was prepared from 3-methylphenethylamine (0.05 g, 0.32 mmol) following the general procedure C as white solid (0.03 g, 62%). ¹H NMR (300 MHz, DMSO-d6) δ 8.62 (s, 1H), 7.41 (d, J=8.85 Hz, 2H), 7.25 (d, J=8.85 Hz, 2H), 7.16-7.22 (m, 1 H), 7.04 (d, J=3.96 Hz, 2H), 7.01 (s, 1 H), 6.14 (t, J=5.65 Hz, 1 H), 3.27-3.33 (m, 2H), 2.70 (t, J=7.16 Hz, 2H), 2.29 (s, 3H). ¹³C NMR (75 MHz, DMSO-d6) δ 154.9, 139.5, 139.3, 137.3, 129.3, 128.4, 128.2, 126.7, 125.6, 124.4, 119.0, 35.7, 21.0. MS (ESI) m/z [M+H]+calcd: 289.1; found: 289.1. 3-(4-Chlorophenyl)1-[2-(2-fluorophenyl)ethyl]urea (61) was prepared from 2-fluorophenethylamine (0.04 ml, 0.32 mmol) following the general procedure C as white solid (0.02 g, 21%). ¹H NMR (300 MHz, DMSO-d6) δ 8.62 (s, 1H), 7.41 (d, J=8.67 Hz, 2H), 7.21-7.35 (m, 4H), 7.11-7.20 (m, 2H), 6.22 (t, J=5.27 Hz, 1H), 3.26-3.33 (m, 2H), 2.79 (t, J=7.06 Hz, 2H). ¹³C NMR (75 MHz, DMSO-d6) δ 154.9, 139.4, 137.8, 132.5, 131.6, 128.4, 126.7, 125.8, 125.7, 124.5, 119.1, 32.6. MS (ESI) m/z [M+H]+calcd: 293.1; found: 293.3.

3-(4-Chlorophenyl)1-[2-(3-fluorophenyl)ethyl]urea (62) was prepared from 3-fluorophenethylamine (0.04 ml, 0.32 mmol) following the general procedure C as white solid (0.02 g, 17%). ¹H NMR (300 MHz, DMSO-d6) δ 8.62 (s, 1H), 7.38-7.44 (m, 2H), 7.30-7.37 (m, 1H), 7.25 (d, J=9.04 Hz, 2H), 7.08 (d, J=8.67 Hz, 2H), 6.99-7.05 (m, 1H), 6.17 (t, J=5.65 Hz, 1H), 3.32-3.40 (m, 2H), 2.77 (t, J=7.06 Hz, 2H). ¹³C NMR (75 MHz, DMSO-d6) 6 154.9, 142.5, 142.4, 139.5, 130.2, 130.1, 128.4, 124.8, 124.8, 124.4, 119.1, 115.5, 115.2, 112.9, 112.7, 35.3. MS (ESI) m/z [M-H]⁻ calcd: 291.1; found: 291.1.

3-(4-Chlorophenyl)1-[2-(4-fluorophenyl)ethyl]urea (63) was prepared from 4-fluorophenethylamine (0.04 ml, 0.32 mmol) following the general procedure C as white solid (0.05 g, 48%). ¹H NMR (300 MHz, DMSO-d6) δ 8.61 (s, 1H), 7.41 (d, J=8.85 Hz, 2H), 7.21-7.31 (m, 4H), 7.09-7.17 (m, 2H), 6.15 (t, J=5.56 Hz, 1H), 3.27-3.33 (m, 2H), 2.74 (t, J=7.06 Hz, 2H). ¹³C NMR (75 MHz, DMSO-d6) 6 162.4, 159.2, 154.9, 139.5, 135.6, 135.6, 130.4, 130.3, 128.4, 124.4, 119.0, 115.1, 114.8, 34.8. MS (ESI) m/z [M-H]⁻ calcd: 291.1; found: 291.0. 3-(4-Chlorophenyl)1-[2-(3,4-difluorophenyl)ethyl]urea (64) was prepared from 3,4-difluorophenethylamine (0.18 g, 1 mmol) following the general procedure C as white solid (0.05 g, 50%). ¹H NMR (300 MHz, DMSO-d6) 6 8.60 (s, 1 H), 7.38-7.43 (m, 2H), 7.28-7.38 (m, 2H), 7.22-7.27 (m, 2H), 7.08 (ddd, J=2.26, 4.10, 6.26 Hz, 1H), 6.16 (t, J=5.65 Hz, 1H), 3.28-3.34 (m, 2H), 2.74 (t, J=6.97 Hz, 2H). ¹³C NMR (75 MHz, DMSO-d6) 6 154.9, 139.4, 137.4, 128.4, 125.5, 125.4, 124.4, 119.1, 117.6, 117.4, 117.2, 117.0, 34.7. MS (ESI) m/z [M-H]⁻ calcd: 291.1; found: 291.1.

3-(4-Chlorophenyl)1-[2-(2,4,6-trifluorophenyl)ethyl]urea (65) was prepared from 136 (0.11 g, 0.6 mmol) following the general procedure C as white solid (0.11 g, 54%). ¹H NMR (300 MHz, DMSO-d6) δ 8.67 (s, 1 H), 7.45 (d, J=8.85 Hz, 2H), 7.30 (d, J=8.85 Hz, 2H), 7.17-7.25 (m, 2H), 6.32 (t, J =5.93 Hz, 1 H), 3.33 (q, J=6.59 Hz, 2H), 2.82 (t, J=6.69 Hz, 2H). ¹³C NMR (75 MHz, DMSO-d6) 6 162.6, 159.3, 154.9, 139.4, 128.6, 128.4, 124.4, 119.8, 119.1, 111.2, 100.3, 30.6, 22.7. MS (ESI) m/z [M+H]+calcd: 329.1; found: 329.2. 3-(4-Chlorophenyl)1-[2-(2,3,4,5,6-pentafluorophenyl)ethyl]urea (66) was prepared from 137 (0.17 g, 0.78 mmol) following the general procedure C as white solid (0.07 g, 23%). ¹H NMR (300 MHz, CDC13) d 7.50 (s, 1H), 7.24-7.27 (m, 2H), 7.18-7.23 (m, 2H), 5.53 (t, J=5.65 Hz, 1H), 3.46 (q, J=6.59 Hz, 2H), 2.93 (t, J=6.69 Hz, 2H). ¹³C NMR (75 MHz, DMSO-d6) δ 155.0, 146.6, 143.5, 143.3, 143.2, 140.5, 139.3, 138.4, 137.2, 137.0, 135.3, 135.1, 128.6, 128.4, 124.6, 119.6, 119.2, 113.4, 113.2, 113.1, 112.9, 38.2, 23.2. MS (ESI) m/z [M+H]+calcd: 365.1; found: 365.5.

3-(4-Chlorophenyl)1-[2-(2-chlorophenyl)ethyl]urea (67) was prepared from 2-chlorophenethylamine (0.05 ml, 0.32 mmol) following the general procedure C as white solid (0.07 g, 17%). ¹H NMR (300 MHz, DMSO-d6) δ 8.61 (s, 1H), 7.39-7.46 (m, 3H), 7.30-7.38 (m, 2H), 7.28 (d, J=1.70 Hz, 4H), 6.24 (t, J=5.56 Hz, 1H), 3.34-3.40 (m, 2H), 2.88 (t, J=7.06 Hz, 2H).

¹³C NMR (75 MHz, DMSO-d6) δ 154.9, 139.5, 136.8, 133.1, 131.0, 129.2, 128.4, 128.1, 127.2, 124.4, 119.1, 33.5. MS (ESI) m/z [M+H]+calcd: 309.1; found: 309.3.

3-(4-Chlorophenyl)1-[2-(3-chlorophenyl)ethyl]urea (68) was prepared from 3-chlorophenethylamine (0.05 ml, 0.32 mmol) following the general procedure C as white solid (0.09 g, 92%). ¹H NMR (300 MHz, CDC13) δ 7.27-7.31 (m, 1H), 7.22-7.25 (m, 3H), 7.17-7.21 (m, 3H), 7.06-7.11 (m, 1H), 6.11 (br. s., 1 H), 4.55 (t, J=5.46 Hz, 1 H), 3.51 (q, J=6.66 Hz, 2H), 2.83 (t, J=6.78 Hz, 2H). ¹³C NMR (75 MHz, DMSO-d6) δ 154.9, 142.1, 139.4, 132.9, 130.1, 128.5, 128.4, 127.4, 126.1, 124.4, 119.1, 35.2. MS (ESI) m/z [M+H]+calcd: 309.1; found: 309.0.

3-(4-Chlorophenyl)1-[2-(2,4-dichlorophenyl)ethyl]urea (69) was prepared from 2,4-dichlorophenethylamine (0.05 ml, 0.32 mmol) following the general procedure C as white solid (0.02 g, 19%). ¹H NMR (300 MHz, DMSO-d6) δ 8.60 (s, 1H), 7.59 (d, J=1.32 Hz, 1H), 7.37-7.43 (m, 4H), 7.25 (d, J=9.04 Hz, 2H), 6.22 (t, J=5.75 Hz, 1H), 3.28-3.34 (m, 2H), 2.86 (t, J=6.97 Hz, 2H). ¹³0 NMR (75 MHz, DMSO-d6) 6 154.9, 139.4, 136.1, 134.1, 132.3, 131.6, 128.6, 128.4, 127.3, 124.4, 119.1, 32.9. MS (ESI) m/z calcd: 341.1; found: 341.4.

3-(4-Chlorophenyl)1-[2-(2-chloro-6-fluorophenyl)ethyl]urea (70) was prepared from 2-chloro-6-fluorophenethylamine (0.06 g, 0.32 mmol) following the general procedure C as white solid (0.02 g, 23%). ¹H NMR (300 MHz, DMSO-d6) δ 8.60 (br. s., 1H), 7.39 (s, 2H), 7.29 (d, J=17.52 Hz, 5H), 6.29 (br. s., 1 H), 3.29-3.37 (m, 2H), 2.87-2.99 (m, 2H). ¹³0 NMR (75 MHz, DMSO-d6) δ 154.9, 139.4, 134.5, 134.4, 129.0, 128.8, 128.4, 125.3, 125.3, 124.4, 119.1, 114.4, 114.1, 96.3, 27.0. MS (ESI) m/z [M+H]+calcd: 327.1; found: 327.3.

3-(4-Chlorophenyl)1-[2-(4-bromophenyl)ethyl]urea (71) was prepared from 4-bromophenethylamine (0.08 g, 0.32 mmol) following the general procedure C as white solid (0.06 g, 50%). ¹H NMR (300 MHz, CDC13) δ 7.43 (d, J=8.29 Hz, 2H), 7.18-7.25 (m, 4H), 7.08 (d, J=8.29 Hz, 2H), 6.23 (s, 1H), 4.61 (br. s., 1H), 3.45-3.55 (m, 2H), 2.74-2.85 (m, 2H). ¹³C NMR (75

MHz, DMSO-d6) δ 154.9, 139.5, 138.9, 131.1, 131.0, 128.4, 124.4, 119.1, 119.0, 35.0. MS (ESI) m/z [M-H]⁻ calcd: 353.1; found: 353.1.

3-(4-Chlorophenyl)1-[2-(4-cyanophenyl)ethyl]urea (72) was prepared from 4-cyanophenethylamine (0.10 g, 0.64 mmol) following the general procedure C as white solid (0.04 g, 22%). ¹H NMR (300 MHz, DMSO-d6) δ 8.73 (s, 1H), 7.78 (d, J=8.29 Hz, 2H), 7.45 (d, J=8.10 Hz, 2H), 7.40 (d, J=9.04 Hz, 2H), 7.24 (d, J=8.85 Hz, 2H), 6.26 (t, J=5.65 Hz, 1 H), 3.35-3.42 (m, 2H), 2.84 (t, J=6.97 Hz, 2H). ¹³C NMR (75 MHz, DMSO-d6) δ 154.9, 145.7, 139.4, 132.2, 129.8, 128.4, 124.4, 119.1, 118.9, 109.0, 35.8. MS (ESI) m/z [M+H]+calcd: 300.1; found: 300.3.

3-(4-Chlorophenyl)-1-{2-[2-(trifluoromethyl)phenyl]ethyl}urea (73) was prepared from 2-trifluoromethylphenethylamine (0.06 ml, 0.32 mmol) following the general procedure C as white solid (0.07 g, 15%). ¹H NMR (300 MHz, DMSO-d6) δ 8.62 (s, 1 H), 7.70 (d, J=7.91 Hz, 1 H), 7.61-7.67 (m, 1 H), 7.51 (d, J=7.72 Hz, 1 H), 7.44-7.48 (m, 1 H), 7.39-7.43 (m, 2H), 7.25 (d, J=9.04 Hz, 2H), 6.32 (t, J=5.75 Hz, 1H), 3.36-3.43 (m, 1H), 2.93 (t, J=7.16 Hz, 2H). ¹³0 NMR (75 MHz, DMSO-d6) δ 154.9, 139.4, 137.8, 132.5, 131.6, 128.4, 126.7, 125.8, 125.7, 124.5, 119.1, 32.6. MS (ESI) m/z [M+H]+calcd: 343.1; found: 343.3.

3-(4-Chlorophenyl)-1-{2-[3-(trifluoromethyl)phenyl]ethyl}urea (74) was prepared from 3-trifluoromethylphenethylamine (0.06 ml, 0.32 mmol) following the general procedure C as white solid (0.08 g, 76%). ¹H NMR (300 MHz, DMSO-d6) δ 8.62 (s, 1 H), 7.52-7.62 (m, 4H), 7.42 (d, J=8.85 Hz, 2H), 7.24 (d, J=8.85 Hz, 2H), 6.21 (t, J=5.56 Hz, 1 H), 3.34-3.44 (m, 2H), 2.86 (t, J=6.97 Hz, 2H). ¹³C NMR (75 MHz, DMSO-d6) 6 155.0, 141.0, 139.4, 132.8, 129.2, 128.4, 125.2, 125.1, 124.5, 122.8, 122.8, 119.1, 40.2, 35.4. MS (ESI) m/z [M+H]+calcd: 343.1; found: 343.3.

3-(4-Chlorophenyl)-1-{2-[4-(trifluoromethyl)phenyl]ethyl}urea (75) was prepared from 4-trifluoromethylphenethylamine (0.05 ml, 0.32 mmol) following the general procedure C as white solid (0.07 g, 64%). ¹H NMR (300 MHz, DMSO-d6) δ 8.61 (s, 1 H), 7.67 (d, J=7.91 Hz, 2H), 7.47 (d, J=7.91 Hz, 2H), 7.40 (d, J=8.85 Hz, 2H), 7.25 (d, J=8.85 Hz, 2H), 6.18 (t, J=5.65 Hz, 1H), 3.37-3.42 (m, 2H), 2.85 (t, J=6.97 Hz, 2H). ¹³C NMR (75 MHz, DMSO-d6) 6 154.9, 144.5, 139.4, 129.5, 128.4, 125.1, 125.1, 125.0, 124.4, 119.1, 35.5. MS (ESI) m/z [M+H]+calcd: 343.1; found: 343.3. 3-(4-Chlorophenyl)-1-[2-(pyridin-4-yl)ethyl]urea (76) was prepared from 4-(2-aminoethyl)pyridine (0.04 ml, 0.32 mmol) following the general procedure

C as white solid (0.03 g, 34%). ¹H NMR (300 MHz, DMSO-d6) δ 8.62 (s, 1H), 8.48 (d, J=5.84 Hz, 2H), 7.40 (d, J=8.85 Hz, 2H), 7.22-7.30 (m, 4H), 6.19 (t, J=5.65 Hz, 1H), 3.35-3.43 (m, 2H), 2.77 (t, J=6.88 Hz, 2H). ¹³C NMR (75 MHz, DMSO-d6) 6 154.9, 149.5, 148.4, 139.4, 128.4, 124.4, 124.2, 119.1, 34.9. MS (ESI) m/z [M+H]+calcd: 276.1; found: 276.1.

3-(4-Chlorophenyl)-1-[2-(pyridin-3-yl)ethyl]urea (77) was prepared from 3-(2-aminoethyl)pyridine (0.04 g, 0.32 mmol) following the general procedure C as white solid (0.08 g, 91%). ¹H NMR (300 MHz, CDC13) δ 8.30-8.38 (m, 2H), 7.53-7.63 (m, 2H), 7.18-7.26 (m, 5H), 5.49 (t, J=5.50 Hz, 1H), 3.52 (q, J=5.71 Hz, 2H), 2.78-2.87 (m, 2H). ¹³C NMR (75 MHz, CHC13) 6 155.6, 149.8, 147.6, 137.5, 136.8, 135.0, 129.1, 128.2, 123.9, 121.0, 40.7, 33.3. MS (ESI) m/z [M+H]+calcd: 276.1; found: 276.1.

3-(4-Chlorophenyl)-1-[2-(pyridin-2-yl)ethyl]urea (78) was prepared from 2-(2-aminoethyl)pyridine (0.04 g, 0.32 mmol) following the general procedure C as white solid (0.08 g, 84%). ¹H NMR (300 MHz, CDC13) δ 8.38 (d, J=4.14 Hz, 1 H), 7.87 (br. s., 1 H), 7.55-7.62 (m, 1 H), 7.10-7.24 (m, 6H), 6.15-6.24 (m, 1H), 3.62 (q, J=5.84 Hz, 2H), 2.94-3.01 (m, 2H). ¹³C NMR (75 MHz,

CDC13) δ 159.6, 156.0, 148.8, 137.8, 136.9, 129.0, 128.0, 123.6, 121.7, 121.3, 39.6, 37.6. MS (ESI) m/z [M+H]+calcd: 276.1; found: 276.2.

1-(4-Chlorophenyl)-3-[2-(5-methylfuran-2-yl)ethyl]urea (79) was prepared from 2-(5-methyl-2-furyl)ethanamine (0.04 ml, 0.32 mmol) following the general procedure C as white solid (0.04 g, 44%). ¹H NMR (300 MHz, CDC13) δ 7.18-7.25 (m, 4H), 6.34 (br. s., 1H), 5.84-5.96 (m, 2H), 4.90 (t, J=5.75 Hz, 1H), 3.51 (q, J=6.22 Hz, 2H), 2.80 (t, J=6.40 Hz, 2H), 2.23 (s, 3H). ¹³C NMR (75 MHz, CDC13) δ 155.2, 151.2, 137.1, 129.2, 128.9, 122.1, 107.2, 106.1, 39.1, 28.6, 13.5. MS (ESI) m/z [M+H]+calcd: 279.1; found: 279.1.

3-(4-Chlorophenyl)1-[2-(4-methylpiperazin-1-yl)ethyl]urea (80) was prepared from (4-methylpiperazin-1-yl)ethanamine (0.05 ml, 0.32 mmol) following the general procedure C as white solid (0.04 g, 43%). ¹H NMR (300 MHz, CD3OD) δ 7.31-7.38 (m, 2H), 7.18-7.24 (m, 2H), 4.83-4.88 (m, 2H), 2.34-2.77 (m, 10H), 2.28 (s, 3H). ¹³C NMR (75 MHz, CD30D) δ 158.0, 140.0, 129.7, 128.1, 121.3, 58.6, 55.8, 53.7, 46.0, 37.8. MS (ESI) m/z [M-1-1]⁻ calcd: 297.1; found: 297.2.

3-(4-Chlorophenyl)-1-[2-(piperidin-1-yl)ethyl]urea (81) was prepared from 1-(2-aminoethyl)piperidine (0.05 ml, 0.32 mmol) following the general procedure C as white solid (0.06 g, 66%). ¹H NMR (300 MHz, CDC13) δ 8.53 (br. s., 1H), 7.24-7.33 (m, 2H), 7.12-7.22 (m, 2H), 6.13 (br. s., 1H), 3.97-4.12 (m, 1H), 3.26-3.39 (m, 2H), 2.35-2.60 (m, 6H), 1.53-1.66 (m, 4H), 1.46 (d, J=4.71 Hz, 2H). ¹³C NMR (75 MHz, CDC13) δ 156.8, 138.1, 128.8, 127.5, 121.0, 77.5, 77.1, 76.6, 58.8, 54.5, 37.3, 25.3, 23.8. MS (ESI) m/z [M+H]+calcd: 282.1; found: 282.3.

3-(4-Chlorophenyl)-1-[2-(morpholin-4-yl)ethyl]urea (82) was prepared from 4-(2-aminoethyl)morpholine (0.04 ml, 0.32 mmol) following the general procedure C as white solid (0.08 g, 82%). ¹H NMR (300 MHz, CDC13) δ 7.56 (br. s., 1H), 7.19-7.32 (m, 4H), 5.52-5.64 (m, 1H), 3.62-3.77 (m, 4H), 3.34 (q, J=5.46 Hz, 2H), 2.36-2.56 (m, 6H). ¹³C NMR (75 MHz, CDC13) δ 156.0, 137.6, 129.0, 128.3, 121.4, 66.8, 58.0, 53.5, 36.9. MS (ESI) m/z [M+H]⁻ calcd: 284.1; found: 284.5.

1-(4-Chlorophenyl)-3-[2-(pyrrolidin1-yl)ethyl]urea (83) was prepared from 1-(2-aminoethyl)pyrrolidine (0.04 ml, 0.32 mmol) following the general procedure C as white solid (0.01 g, 12%). ¹H NMR (300 MHz, CDC13) δ 8.67 (br. s., OH), 6.99-7.33 (m, 4H), 5.96 (br. s., 1H), 3.32 (q, J=5.15 Hz, 2H), 2.63-2.72 (m, 2H), 2.49-2.63 (m, 4H), 1.72-1.89 (m, 4H). ¹³C NMR (75 MHz, CDC13) δ 157.0, 138.3, 128.8, 127.5, 120.8, 56.7, 54.1, 39.7, 23.6. MS (ESI) m/z [M+H]+calcd: 268.1; found: 268.1.

N-(2-{[(4-Chlorophenyl)carbamoyl]amino}ethyl)acetamide (84) was prepared from N-acetylethylenediamine (0.03 ml, 0.32 mmol) following the general procedure C as white solid (0.02 g, 24%). ¹H NMR (300 MHz, DMSO-d6) δ 8.69 (s, 1 H), 7.93 (br. s., 1 H), 7.42 (d, J=8.85 Hz, 2H), 7.25 (d, J=8.85 Hz, 2H), 6.20 (br. s., 1 H), 3.03-3.22 (m, 4H), 1.81 (s, 3H). ¹³0 NMR (75 MHz, DMSO-d6) δ 169.4, 155.1, 139.5, 128.4, 124.4, 119.1, 50.2, 30.6, 22.6. MS (ESI) m/z [M+H]+calcd: 256.1; found: 256.4.

3-(4-Chlorophenyl)-1-(2-methyl-2-phenylpropyl)urea (38) was prepared from 133 (0.10 g, 0.56 mmol) following the general procedure C as white solid (0.14 g, 81%). ¹H NMR (300 MHz, CDC13) δ 7.34 (d, J=4.14 Hz, 4H), 7.20-7.25 (m, 1H), 7.16-7.20 (m, 2H), 7.05-7.11 (m, 2H), 6.14 (br. s., 1H), 4.37 (br. s., 1 H), 3.45 (d, J=6.03 Hz, 2H), 1.35 (s, 6H). ¹³C NMR (75 MHz, CDC13) δ 155.4, 146.6, 137.2, 129.0, 128.6, 128.5, 126.3, 126.0, 121.6, 51.7, 38.9, 26.6. MS (ESI) m/z [M+H]+calcd: 303.1; found: 303.2. 3-(4-Chlorophenyl)-1-(2,2-difluoro-2-phenylethyl)urea (39) was prepared from 134 (0.06 g, 0.29 mmol) following the general procedure C as white solid (0.05 g, 52%). ¹H NMR (300 MHz, DMSO-d6) δ 8.72 (s, 1H), 7.49-7.62 (m, 5H), 7.36-7.43 (m, 2H), 7.24-7.30 (m, 2H), 6.57 (t, J=6.22 Hz, 1H), 3.88 (dt, J=6.22, 14.98 Hz, 2H). ¹³C NMR (75 MHz, DMSO-d6) δ 154.6, 139.0, 135.0, 134.7, 134.3, 130.3, 128.6, 128.5, 125.2, 125.1, 125.1, 124.9, 124.2, 121.0, 119.2, 117.8, 45.1, 44.6, 44.2. MS (ESI) m/z [M+H]+calcd: 311.1; found: 311.2.

3-(4-Chlorophenyl)1-(2-methyl1-phenylpropan-2-yl)urea (40) was prepared from 139 (0.06 g, 0.38 mmol) following the general procedure D as white solid (0.07 g, 57%). ¹H NMR (300 MHz, CDC13) δ 7.20-7.27 (m, 5H), 7.12-7.19 (m, 4H), 6.21 (s, 1H), 4.41 (s, 1H), 3.03 (s, 2H), 1.33 (s, 6H). ¹³C NMR (75 MHz, CDC13) δ 154.4, 138.1, 137.4, 130.6, 129.2, 128.7, 128.1, 126.4, 122.0, 53.7, 45.5, 27.9. MS (ESI) m/z [M+H]+calcd: 303.1; found: 303.2.

1-(4-Chlorophenyl)-3-[(1-phenylcyclopropyl)methyl]urea (41) was prepared from (1-phenylcyclopropyl)methylamine (0.03 g, 0.2 mmol) following the general procedure C as white solid (0.04 g, 68%). 1H NMR (300

MHz, CDC13) δ 7.28-7.39 (m, 4H), 7.10-7.24 (m, 5H), 6.22 (br. s., 1 H), 4.70 (br. s., 1 H), 3.43 (d, J=5.46 Hz, 2H), 0.89 (s, 4H). MS (ESI) m/z [M+H]+calcd: 301.1; found: 301.4.

3-(1-Benzylcyclopropyl)1-(4-chlorophenyl)urea (42) was prepared from (1-benzylcyclopropyparnine hydrochloride hydrate (0.04 g, 0.2 mmol) following the general procedure C as white solid (0.03 g, 48%). ¹H NMR (300 MHz, CDC13) δ 7.29-7.34 (m, 2H), 7.27-7.28 (m, 1 H), 7.20-7.24 (m, 2H), 7.17-7.20 (m, 2H), 7.10-7.15 (m, 2H), 6.34 (s, 1H), 4.86 (s, 1H), 2.86 (s, 2H), 0.91-0.98 (m, 4H). ¹³C NMR (75 MHz, CDC13) δ 153.5, 136.9, 135.5, 127.9, 127.3, 127.2, 126.8, 125.5, 119.6, 41.5, 33.3, 13.0. MS (ESI) m/z [M+H]+calcd: 301.1; found: 301.4. trans1-(4-Chlorophenyl)-3-[(2-phenylcyclopropyl)methyl]urea (43) was prepared from 135 (0.08 g, 0.45 mmol) following the general procedure C as white solid (0.07 g, 51%). ¹H NMR (300 MHz, DMSO-d6) δ 8.62 (s, 1H), 7.38-7.46 (m, 2H), 7.20-7.29 (m, 4H), 7.11-7.15 (m, 1H), 7.04-7.10 (m, 2H), 6.32 (t, J=5.56 Hz, 1H), 3.16-3.28 (m, 1H), 2.99-3.11 (m, 1H), 1.79-1.88 (m, 1H), 1.21-1.35 (m, 1H), 0.83-0.96 (m, 2H). ¹³C NMR (75 MHz, DMSO-d6) δ 155.0, 142.8, 139.5, 128.4, 128.1, 125.5, 125.2, 124.4, 119.1, 42.9, 23.4, 21.3, 14.3. MS (ESI) m/z [M+H]+calcd: 301.1; found: 301.2.

### Example 2

### In Vitro Assays

Calcium Mobilization Assay: CHO-RD-HGA16 cells (Molecular Devices, San Jose, Calif., United States of America) stably expressing the human CB₁ receptor were plated into 96-well black-walled assay plates at 25,000 cells/well in 100 pL of Ham's F12 (supplemented with 10% fetal bovine serum, 100 units of penicillin/streptomycin, and 100 pg/mL Normocin) and incubated overnight at 37° C., 5% CO₂. Calcium 5 dye (Molecular Devices, San Jose, Calif., United States of America) was reconstituted according to the manufacturer's instructions. The reconstituted dye was diluted 1:40 in prewarmed (37° C.) assay buffer (1x HBSS, 20 mM HEPES, 2.5 mM probenecid, pH 7.4 at 37° C.). Growth medium was removed, and the cells were gently washed with 100 pL of prewarmed (37° C.) assay buffer. The cells were incubated for 45 min at 37° C., 5% CO2 in 200 pL of the diluted Calcium 5 dye solution. For antagonist assays to determine IC₅₀ values, the EC₈₀ concentration of CP55,940 was prepared at 10x the desired final concentration in 0.25% BSA/0.5% DMSO/0.5% EtOH/assay buffer, aliquoted into 96-well polypropylene plates, and warmed to 37° C. Serial dilutions of the test compounds were prepared at 10x the desired final concentration in 2.25% BSA/4.5% DMSO/4.5% EtOH/assay buffer. After the dye loading incubation period, the cells were pretreated with 25 pL of the test compound serial dilutions and incubated for 15 min at 37° C. After the pretreatment incubation period, the plate was read with a FLIPR Tetra (Molecular Devices, San Jose, Calif., United States of America). Calcium-mediated changes in fluorescence were monitored every 1 s over a 90 s time period, with the Tetra adding 25 pL of the CP55,940 EC₈₀ concentration at the 10s time point (excitation/emission: 485/525 nm). Relative fluorescence units (RFU) were plotted against the log of compound concentrations. For agonist screens, the above procedure was followed except that cells were pretreated with 2.25% BSA/4.5% DMSO/4.5% EtOH/assay buffer and the Tetra added single concentration dilutions of the test compounds prepared at 10x the desired final concentration in 0.25% BSA/0.5% DMSO/0.5% EtOH/assay buffer. Test compound RFUs were compared to the CP55,940 Emax RFUs to generate % Eₘₐₓ values. For the CB₂ agonist and antagonist assays, the same procedures were followed except that stable human CB₂-CHO-RD-HGA16 cells were used.

[³⁵S]GTPγS Binding Assay: For receptor signaling, membranes (10 μg protein) from either ICR mouse cerebellum mice (6-8 weeks old; Enviga

International, Indianapolis, Indiana, United States of America) or HEK cells stably expressing CB1 receptor were preincubated in assay buffer for 10 min with 3 units/ml adenosine deaminase then incubated for 60 min at 30° C. with 30 μM GDP and 0.1 nM [³⁵S]GTPyS (Perkin Elmer Life Sciences, Boston, Mass., United States of America). Non-specific binding was determined by adding 30 μM unlabeled GTPyS. Concentration response curves for allosteric modulators were conducted in the presence of CP55,940 (100 nM or 1 μM) to calculate 1050 values.

cAMP Assay: The cAMP assay was performed as previously described. See Cawston et al. J. Med. Chem. 2015, 58, 5979-5988. Briefly, forskolin (FSK)-stimulated cyclic adenosine monophosphate (cAMP) production was measured in real-time using a transfected bioluminescence resonance energy transfer (BRET) cAMP sensor. The plasmid encodes a cAMP binding domain (Epac1) flanked by yellow fluorescent protein (YFP) and Renilla Luciferase (RLuc) assay, the latter of which can oxidize coelenterazine H and produce a photon as a byproduct. When cAMP is bound to the Epac1 domain, it separates RLuc and YFP so only Rluc emits a photon at a wavelength of 460 nm. When cAMP is not bound, RLuc can excite YFP, emitting light at wavelength 535 nm. A plate reader measures both wavelengths and their ratio, 460/535, is calculated to quantify cAMP levels where increases in the ratio indicate increases in cAMP. Human Embryonic Kidney 293 (HEK293) cells stably transfected with the human cannabinoid type-1 (C131) were maintained at 37° C. at 5% CO2 and seeded in 100 mM dishes for transfection. The next day, cells were given fresh growth media and transfected with 5 pg of pcDNA3L-His-CAMYEL using linear polyethyleneimine (25 kDa, Polysciences, Warrington, Pennsylvania, United States of America) in 1:6 DNA:PEI (ATCC, Manassas, Virginia, United States of America) ratio. The next day, cells were lifted using 1 mM EDTA in PBS and spun down at 200xg for 5 min. The supernatant was removed, and cells were resuspended in growth media and plated on poly-D-lysine (Sigma Aldrich, St. Louis, Missouri, United States of America) coated white 96 well plates at 60,000 cells per well, filling 2 columns of 8 wells each per plate, i.e. 8 samples in duplicate per plate (Perkin Elmer, Waltham, Massachusetts,

United States of America). The following day, media was removed, cells were rinsed with PBS and buffers/reagents/drugs added as following: At 0 min, 175 of stimulation buffer (5 mg/ml bovine serum albumin in HBSS including Ca²⁺ and Mg²⁺); at 10 min, 25 μL of allosteric modulators added; at 15 min: 25 μL coelenterazine added (5 μM final); at 25 min, 25 μL of forskolin (10 final) with or without CP55,940 (100 nM final) added. Immediately following addition of forskolin and the probe agonist CP55,940, the luminescence is measured at 460 nm and 535 nm simultaneously for 1 s per well for 22 min at 37° C. using a Clariostar (BMG Labtech, Ortenberg, Germany). Ratio of 460/535 is calculated for each time point and plotted across time and area under the curve analysis is conducted for each replicate and averaged by condition/day where each day serves as an independent experiment. Data were calculated as %FSK using the formula [(sample-basal)/(forskolin-basal)×100]. IC₅₀ values were calculated from these normalized concentration-response data using Prism 6 (Graphpad Software, San Diego, Calif., United States of America) using 3 parameter non-linear regression. Data are plotted as mean of at least N=3 independent experiments either normalized to forskolin (concentration response data) or the calculated 460/535 BRET ratio (time course data).

Data Analysis: For calcium mobilization experiments, data were fit to a three-parameter logistic curve to generate IC₅₀ values (GraphPad Prism 6.0, Graphpad Software, San Diego, Calif., United States of America). For [³⁵S]GTPyS experiments, data were normalized to maximal CP55,940 (100 nM) stimulation in the absence of test compound (i.e., vehicle=100%). Curve fits were accomplished using GraphPad Prism 6.0 (Graphpad Software, San Diego, Calif., United States of America) and data were fit to three-parameter nonlinear regression, with bottom and top constrained to >0 and =100, respectively, for IC₅₀ calculation.

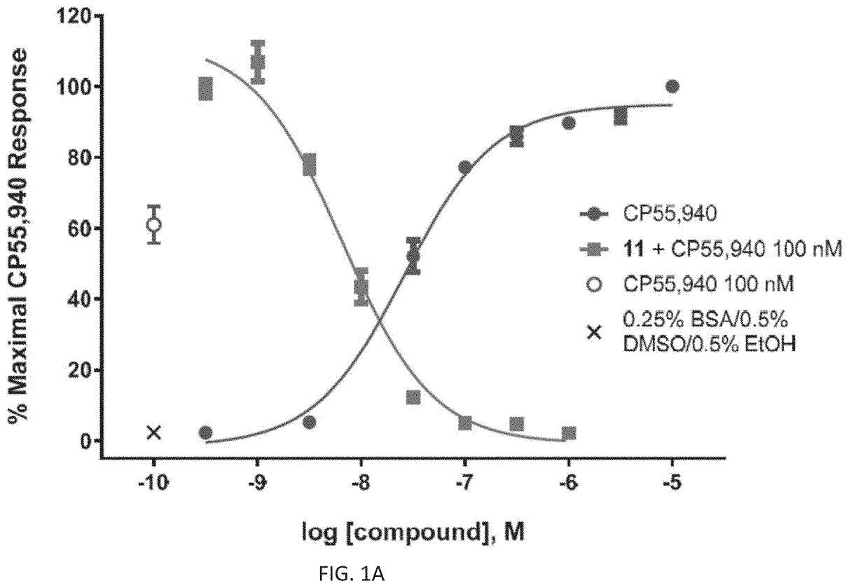

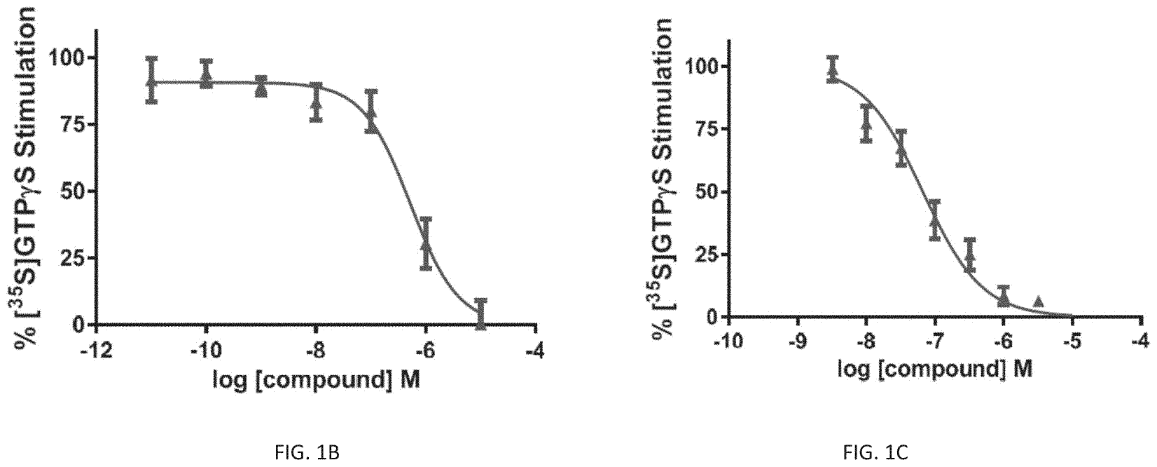

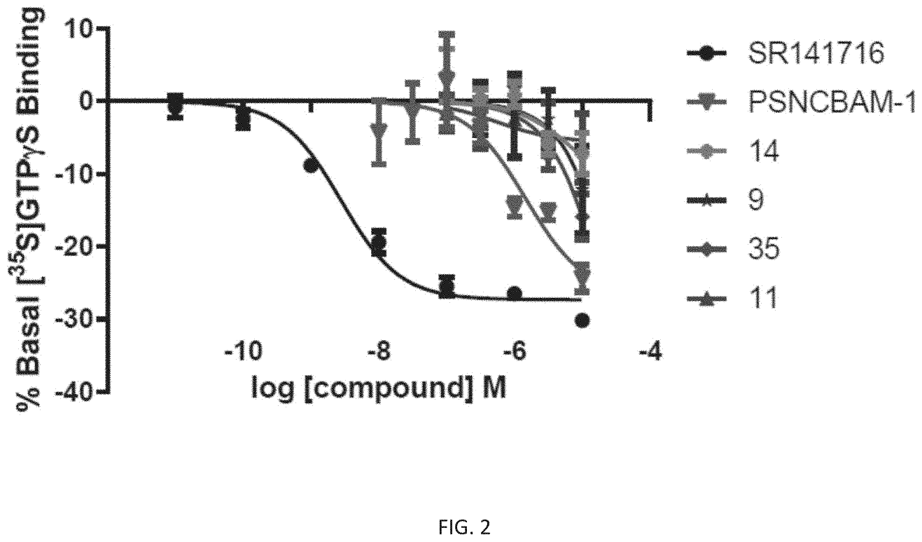

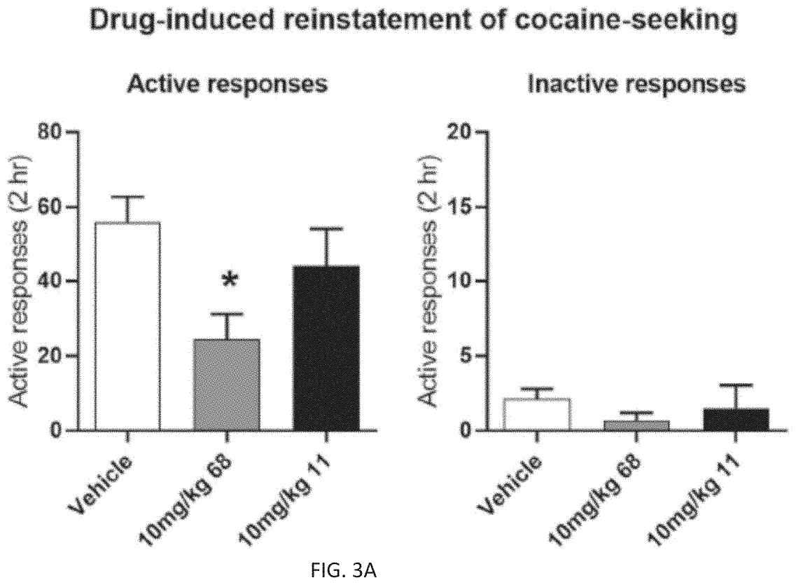

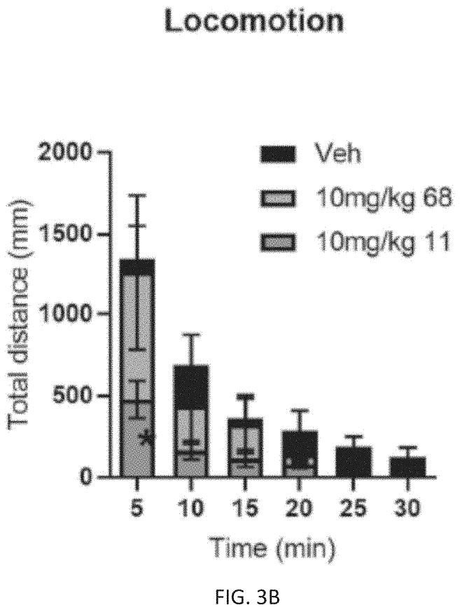

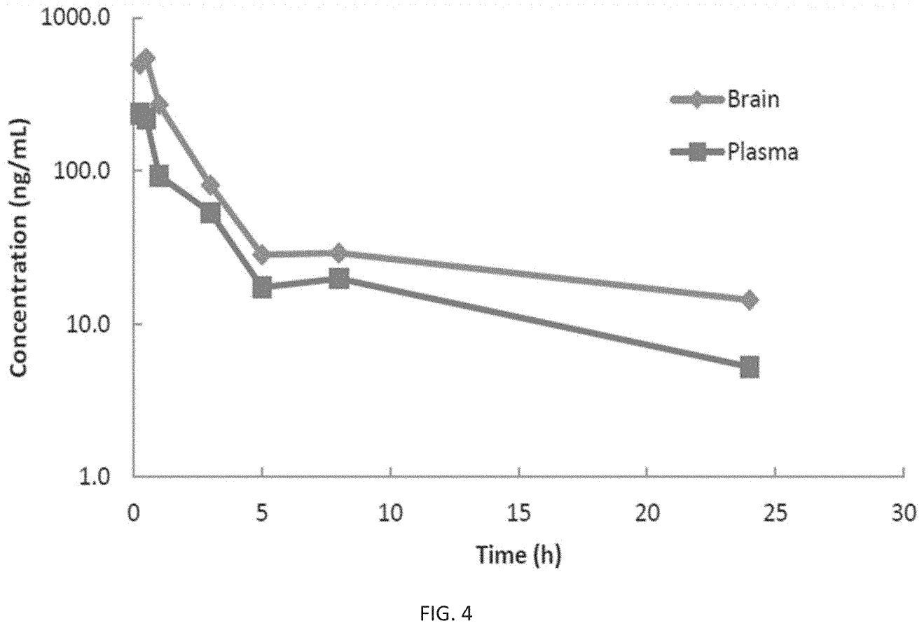

Results: The presently disclosed compounds were characterized in the calcium mobilization assay using CHO cells overexpressing human CB1R and the [³⁵S]GTPyS binding assay in HEK cells overexpressing human CB1R as described previously. Results for compound 11 are shown in FIGS. 1A and 1B. Some compounds were also assessed in the [³⁵S]GTPγS binding assay in mouse cerebellum which has a high expression of CB1R. Results for compound 11 are shown in FIG. 10. In addition, Table 1 shows the 105o values of compounds 2 and 6-17 against the EC₅₀ concentration of CP55,940 (100 nM) in the three assays.

TABLE 1
Allosteric Modulating Activities of Compounds 2 and 6-17 in the
CB1R Calcium Mobilization and [³⁵S]GTPγS Binding Assays.

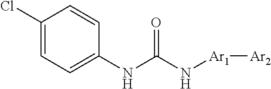

| | | | hCB1R | |
|---|---|---|---|---|
| | | | Calcium | hCB1R |
| | | | assay | [³⁵S]GTPγS |
| | | | IC₅₀ | binding assay |
| Compound | Ar₁ | Ar₂ | (nM)^{a} | IC₅₀ (nM)^{b} |
| 2 | 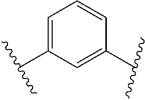 [SMILES: *c1cccc(*)c1] | 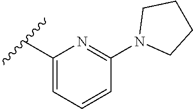 [SMILES: *c1cccc(N2CCCC2)n1] | 33 ± 8 | 455 (307-673) |
| 6 | 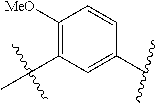 [SMILES: *c1ccc(OC)c(*)c1] | 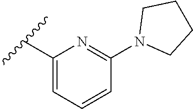 [SMILES: *c1cccc(N2CCCC2)n1] | 1268 ± 424 | >10,000 |
| 7 | 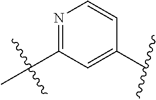 [SMILES: *c1ccnc(*)c1] | Ph | 156 ± 71 | >10,000 |
| 8 | 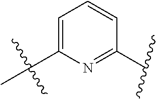 [SMILES: *c1cccc(*)n1] | Ph | 3531 ± 244 | >10,000 |
| 9 | 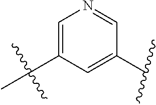 [SMILES: *c1cncc(*)c1] | Ph | 292 ± 55 | >10,000 |
| 10 | 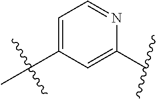 [SMILES: *c1ccnc(*)c1] | Ph | 233 ± 34 | 2770 (1830-4190) |
| 11 | 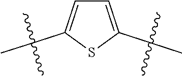 [SMILES: *c1ccc(*)s1] | Ph | 7 ± 1 | 524 (283-969) |
| 12 | 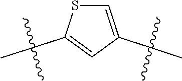 [SMILES: *c1csc(*)c1] | Ph | 37 ± 2 | 667 (417-1060) |
| 13 | 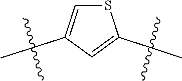 [SMILES: *c1csc(*)c1] | Ph | 60 ± 8 | 888 (621-1270) |
| 14 | 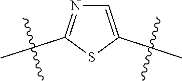 [SMILES: *c1cnc(*)s1] | Ph | 79 ± 10 | 604 (348-1050) |
| 15 | 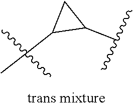 [SMILES: *C1CC1*] | Ph | 1218 ± 188 | >10,000 |
| 16 | 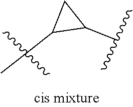 [SMILES: *C1CC1*] | Ph | >10,000 | >10,000 |
| 17 | 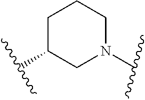 [SMILES: *[C@@H]1CCCN(*)C1] | Ph | 3876 ± 141 | >10,000 |

^{a}Values are the mean ± SEM of at least three independent experiments in duplicate.
^{b}Values are expressed as mean (95% confidence interval) from at least three independent experiments in duplicate.

As diarylurea 2 possesses a flat structure, resulting in tight packing and limited aqueous solubility, a methoxy group was introduced at the 2-position of the middle phenyl ring (6) to introduce steric hindrance, thus preventing the planar structure and disrupting the tight packing. Unfortunately, the allosteric modulating activity was weakened (δ, 1050=1268 nM).

As previously shown (see Nguyen et al., J. Med. Chem. 2017, 60, 7410-7424), the pyrrolidinyl ring of compound 2 is not required for activity.

Thus, to simplify the synthesis effort, the pyrrolidinyl ring was removed when replacing the middle phenyl ring with other aromatic heterocycles such as pyridine, thiophene, and thiazole. Three pyridinyl analogues (7, 9, 10) exhibited a modest drop in activity whereas the activity of the 2,6-pyridinyl analogue (8) was more diminished. Interestingly, the five membered ring analogues, i.e., the thiophenes and thiazole, displayed activities better than their six-membered pyridine counterparts. In particular, the 2,5-thiophenyl analogue (11, IC₅₀=7 nM) had significantly improved activity compared to 2. When the middle phenyl ring was replaced with non-aromatic cyclic rings such as cyclopropyl (15 and 16) or piperidinyl (17), the allosteric modulating activity was diminished.

The results from the [³⁵S]GTPyS binding assay of compound 6-17 against human CB1R were in relatively good agreement with the calcium mobilization assays, albeit the potencies are generally lower. Generally, compounds with weak activities (6, 8, 15, 16, 17) in the calcium assay were also inactive in the [³⁵S]GTPyS binding assay. The pyridinyl analogues (7, 9, and 10) with moderate activities in the calcium assay demonstrated no or weak activities in the [³⁵S]GTPyS binding assay. The five membered ring analogues still maintained good activities in the [³⁵S]GTPyS binding assay comparable to that of 2.

Some differences were observed between assays and species. For example, 7 had no activities in both [³⁵S]GTPyS binding assays but displayed a moderate activity in the calcium assay. 11 demonstrated better potencies in the calcium and [³⁵S]GTPyS binding assay against mouse CB1R than 2 but comparable potency in the [³⁵S]GTPyS binding assay against human CB1R. On the other hand, 12 had comparable potencies in the calcium and [³⁵S]GTPyS binding assay against human CB1R but weaker potency in the [³⁵S]GTPyS binding assay against mouse CB1R. Lastly, 15 had weak activities in the calcium and the [³⁵S]GTPyS binding assay against mouse CB1R without any effect in the [³⁵S]GTPyS binding assay against human CB1R.

TABLE 2
Allosteric Modulating Activities of Compounds 18-35 in the CB1R
Calcium Mobilization and [³⁵S]GTPγS Binding in HEK cells stably
expressing human CB1R Assays.

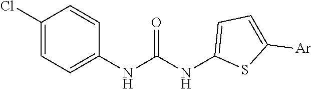

| | | | hCB1R |
|---|---|---|---|
| | | hCB1R | [³⁵S]GTPγS |
| | | Calcium assay | binding assay |
| Compound | Ar | IC₅₀ (nM)^{a} | IC₅₀ (nM)^{b} |
| 18 | 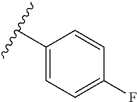 [SMILES: *c1ccc(F)cc1] | 40 ± 2 | 537 (319-904) |
| 19 | 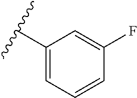 [SMILES: *c1cccc(F)c1] | 39 ± 6 | 425 (281-644) |
| 20 | 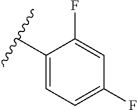 [SMILES: *c1ccc(F)cc1F] | 22 ± 4 | 84 (45-157) |
| 21 | 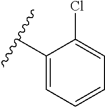 [SMILES: *c1ccccc1Cl] | 21 ± 2 | 272 (181-410) |
| 22 | 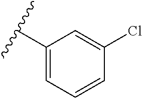 [SMILES: *c1cccc(Cl)c1] | 40 ± 7 | 553 (326-937) |
| 23 | 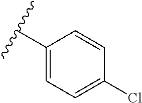 [SMILES: *c1ccc(Cl)cc1] | 43 ± 7 | 774 (506-1180) |
| 24 | 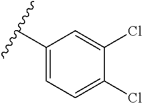 [SMILES: *c1ccc(Cl)c(Cl)c1] | 111 ± 14 | 1720 (941-3130) |
| 25 | 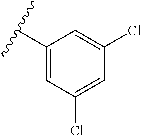 [SMILES: *c1cc(Cl)cc(Cl)c1] | 92 ± 12 | 6330 (4350-9200) |
| 26 | 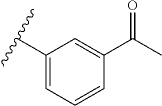 [SMILES: *c1cccc(C(C)=O)c1] | 108 ± 17 | 3430 (2150-5490) |
| 27 | 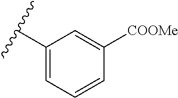 [SMILES: *c1cccc(C(=O)OC)c1] | 345 ± 48 | 2350 (1600-3460) |
| 28 | 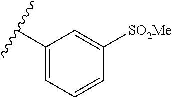 [SMILES: *c1cccc(S(C)(=O)=O)c1] | 501 ± 58 | 3230 (2370-4400) |
| 29 | 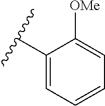 [SMILES: *c1ccccc1OC] | 58 + 5 | 2020 (1410-2890) |
| 30 | 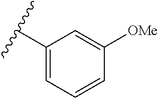 [SMILES: *c1cccc(OC)c1] | 61 + 2 | 3280 (2390-4520) |
| 31 | 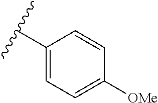 [SMILES: *c1ccc(OC)cc1] | 31 ± 2 | 449 (278-725) |
| 32 | 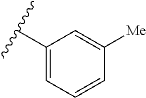 [SMILES: *c1cccc(C)c1] | 48 ± 8 | 1180 (849-1650) |
| 33 | 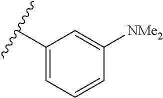 [SMILES: *c1cccc(N(C)C)c1] | 89 ± 14 | 5020 (2800-9010) |
| 34 | 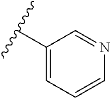 [SMILES: *c1cccnc1] | 107 ± 6 | 298 (184-484) |
| 35 | 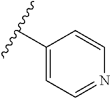 [SMILES: *c1ccncc1] | 79 ± 13 | 263 (133-522) |

^{a}Values are the mean ± SEM of at least three independent experiments in duplicate.
^{b}Values are expressed as mean (95% confidence interval) from at least three independent experiments in duplicate.

With the promising results of the thiophene 11 exhibiting better or equipotent activities in all three assays described above, a focused series of compounds was prepated to investigate the substituent effect on the phenyl ring of the thiophene analogue and these compounds were screened in the calcium and [³⁵S]GTPyS binding assay against human CB1R. As shown in table 2, above, the presence of one or two fluoro substituent(s) and one chloro group (18, 19, 20-23) resulted in a comparable potency to 2 in the calcium assay. However, inclusion of two chloro groups as in 3,4-dichloro and 3,5-dichloro analogues (24 and 25) slightly weakened the activity. Addition of electron-withdrawing groups such as acetyl, methoxycarbonyl, or methylsulfonyl at the 3-position also dampened the activity (26, 27, and 28). Among three positional isomeric methoxy analogues, 4-methoxy analogue (31) was the most potent modulator, whereas the other two analogues exhibited slightly lower potency. The smaller 3-methyl group (32) was more potent than the bigger 3-N,N-dimethylamino group (33). The two pyridinyl analogues (34 and 35) were also active, although their potencies were slightly lower than phenyl counterparts. Overall, these results indicate that small substituents are better tolerated on the phenyl rings than the bigger groups. The presence of a heteroatom is also tolerated, albeit resulting in a slight reduction of activity.

The results from the [³⁵S]GTPyS binding assay were relatively consistent with those from the calcium assay. Compounds with equipotent activities to 2 in the calcium assay also displayed comparable activities in the [³⁵S]GTPyS binding assay (18, 19, 21, 22, 23, and 31). Compounds with weaker activities in the calcium assay also showed weak activities in the [³⁵S]GTPyS binding assay (24-29, and 31-33). Several compounds demonstrated better activities in the [³⁵S]GTPyS binding assay than in the calcium assay. For example, the two pyridinyl analogues (34 and 35) showed comparable potencies to 2 in the [³⁵S]GTPyS binding assay while they exhibited weaker activities in the calcium assay. Fascinatingly, the 2,4-difluoro analogue (20) demonstrated significantly better potencies in the [³⁵S]GTPyS binding assay compared to 2 (20, IC₅₀=84 nM vs 2, IC₅₀=455 nM).

TABLE 3
Activities of select CB1 allosteric modulators in the calcium mobilization,
human and mouse CB1R [³⁵S]GTPγS binding assays, and cAMP assay.

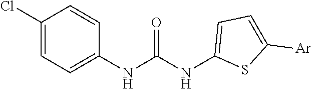

| | | hCB1R | hCB1R | mCB1R | mCB1R | hCB1R |
|---|---|---|---|---|---|---|
| | | Calcium | GTPγS | GTPγS | GTPγS | cAMP |
| | | IC₅₀ (nM) | IC₅₀ (nM) | IC₅₀ (nM) | IC₅₀ (nM) | IC₅₀ (nM) |
| Com- | Structure | [CP] = 100 | [CP] = 100 | [CP] = 100 | [CP] = 1 | [CP] = 100 |
| pound | Ar | nM | nM | nM | uM | nM |
| 2 | | 33 ± 8 | 455 | 244 | 127 | 2620 |
| | | | (307-673) | (158-374) | (74-220) | (1690-4070) |
| 11 | 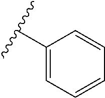 [SMILES: *c1ccccc1] | 7 ± 1 | 524 (283-969) | 63 (42-92) | N.D. | 1760 (1260-2440) |
| 18 | 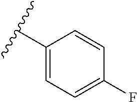 [SMILES: *c1ccc(F)cc1] | 40 ± 2 | 537 (319-904) | 174 (78-399) | 145 (113-191) | N.D. |
| 20 | 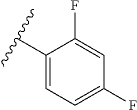 [SMILES: *c1ccc(F)cc1F] | 22 ± 4 | 84 (45-157) | 138 (39-468) | 48 (23-103) | 2290 (1460-3590) |
| 21 | 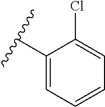 [SMILES: *c1ccccc1Cl] | 21 ± 2 | 272 (181-410) | 363 (159-813) | 204 (96-447) | 1190 (675-2100) |
| 25 | 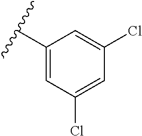 [SMILES: *c1cc(Cl)cc(Cl)c1] | 92 ± 12 | 6330 (4350-9200) | 3311 (1950-5630) | 2399 (1080-5380) | >10,000 |
| 30 | 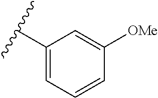 [SMILES: *c1cccc(OC)c1] | 61 + 2 | 3280 (2390-4520) | 3020 (1320-7080) | 1622 (1260-2140) | 6350 (4290-9410) |
| 31 | 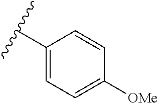 [SMILES: *c1ccc(OC)cc1] | 31 ± 2 | 449 (278-725) | 110 (48-252) | 120 (76-195) | 1850 (1150-2950) |
| 33 | 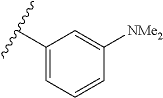 [SMILES: *c1cccc(N(C)C)c1] | 89 ± 14 | 5020 (2800-9010) | 3467 (1450-8320) | 3311 (1950-5630) | >10,000 |
| 35 | 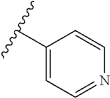 [SMILES: *c1ccncc1] | 79 ± 13 | 298 (184-484) | 204 (67-617) | 120 (76-191) | 1800 (1160-2770) |

Select compounds were evaluated in the CP55,940-induced [³⁵S]GTPyS binding assays using mouse cerebellum membrane at two CP55,940 concentrations, 100 nM and 1μM. Some variations in potencies ranking in the 3 assays were observed. See Table 3, above. 11 showed better potencies than 2 in the calcium and mCB1R GTPyS assays, but comparable potency in the hCB1R GTPyS assay. 18 and 31 had comparable potencies across three assays compared to 2. 20 had better potencies in the calcium and hCB1R GTPyS, but comparable potency to 2 in the mGTPyS assay. 21 demonstrated better potency in the calcium assay and comparable potencies in the two GTPyS assays. Compounds 25, 30, and 33 were slightly less potent than 1 in the calcium assay but demonstrated weak activities in the two GTPyS assays. On the other hand, although 35 exhibited slightly weaker potency in the calcium assay, it displayed similar potencies in the two GTPyS assays.

Interestingly, the allosteric modulatory activities appeared to be more potent at the higher CP55,940 concentration (i.e., 1 μM) in the [³⁵S]GTPyS binding assays using mouse cerebellum membrane. The most significant shift was observed with 20. At 100 nM CP55,940, its IC₅₀ value was 190 nM, which was reduced to 57 nM at 100 nM CP55,940. 35 also exhibited a change in

IC₅₀ values, from 287 nM to 129 nM. This shift in inhibitory potencies reflects positive cooperativity characteristics of these PAM-antagonists.

Representative compounds were assessed in the real-time kinetic BRET CAMYEL cAMP assay. In HEK-hCB1 cells, forskolin (5 μM) induced significant cAMP production at reached plateau after 5 min. The level of cAMP production was inhibited by the agonist CP55,940 (10 nM). All the tested CB1 allosteric modulators attenuated CP55,940-inhibited cAMP production immediately without any “lag” time observed with some of the indole-based analogues. See Cawston et al., J. Med. Chem. 2015, 58, 5979-5988. Intriguingly, except for 21, none of the tested diarylurea-based compounds displayed inverse agonism at concentrations up to 10 μM, unlike 1.

All compounds were screened in the calcium mobilization assay for agonist activity at the CB1R; no significant agonist effects (<30% of CP55,940 Emax, Supporting Information) were observed for any of the compounds. All of these compounds were also screened for agonist and antagonist activity at the CB2R to determine receptor subtype selectivity. None of the compounds had significant CB2R agonist activity (<10% of CP55,940 Emax). All compounds had no significant CB2R antagonist activities (<50% inhibition of CP55,940 ECK) concentration at 10 μM or IC₅₀ values >10 μM).

TABLE 4
Allosteric Modulating Activities of Compounds 36-43 in the hCB1R
Calcium Mobilization and mCB1R [³⁵S]GTPγS Binding Assays.

| | | | mCB1R |
|---|---|---|---|
| | | hCB1R | [³⁵S]GTPγS |
| | | Calcium | binding |
| | | assay | assay |
| Compound | Structure | IC₅₀ (nM)^{a} | IC₅₀ (nM)^{b} |
| 36 | 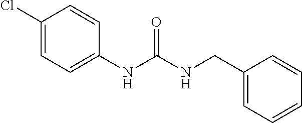 [SMILES: O=C(NCc1ccccc1)Nc1ccc(Cl)cc1] | >10,000 | N.D. |
| 37 | 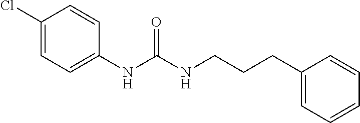 [SMILES: O=C(NCCCc1ccccc1)Nc1ccc(Cl)cc1] | 3703 ± 481 | N.D. |
| 38 | 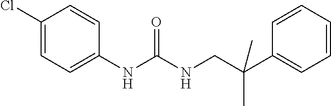 [SMILES: CC(C)(CNC(=O)Nc1ccc(Cl)cc1)c1ccccc1] | >10,000 | N.D. |
| 39 | 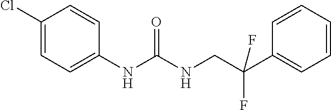 [SMILES: O=C(NCC(F)(F)c1ccccc1)Nc1ccc(Cl)cc1] | 1576 ± 328 | N.D. |
| 40 | 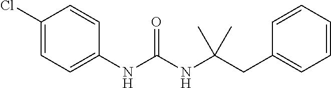 [SMILES: CC(C)(Cc1ccccc1)NC(=O)Nc1ccc(Cl)cc1] | >10,000 | N.D. |
| 41 | 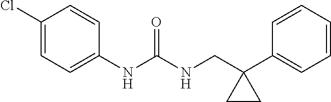 [SMILES: O=C(NCC1(c2ccccc2)CC1)Nc1ccc(Cl)cc1] | 5162 ± 1148 | N.D. |
| 42 | 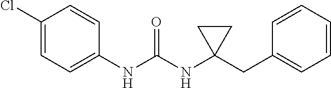 [SMILES: O=C(Nc1ccc(Cl)cc1)NC1(Cc2ccccc2)CC1] | >10,000 | N.D. |
| 43 | 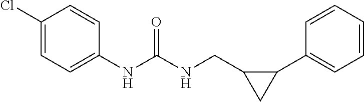 [SMILES: O=C(NCC1CC1c1ccccc1)Nc1ccc(Cl)cc1] | 2611 ± 548 | N.D. |

^{a}Values are the mean ± SEM of at least three independent experiments in duplicate.
^{b}Values are expressed as mean (95% confidence interval) from at least three independent experiments in duplicate.

TABLE 5
Allosteric Modulating Activities of Compounds 44-75 in the hCB1R
Calcium Mobilization and mCB1R [³⁵S]GTPγS Binding Assays.

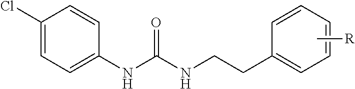

| | | | mCB1R |
|---|---|---|---|
| | | hCB1R | [³⁵S]GTPγS binding |
| | | Calcium assay | assay |
| Compound | Structure | IC₅₀ (nM)^{a} | IC₅₀ (nM)^{b} |
| 44 | H | 193 ± 35 | N.D. |
| 45 | 4-tBu | 573 ± 105 | N.D. |
| 46 | 4-Ph | 897 ± 167 | N.D. |
| 47 | 4-Cl | 164 ± 15 | N.D. |
| 48 | 4-NO₂ | 87 ± 12 | 780 (348-1750) |
| 49 | 3-OMe,4-OH | >10,000 | N.D. |
| 50 | 3-NMe₂ | 1682 ± 284 | 592 (21-16430) |
| 51 | 4-NMe₂ | 323 ± 88 | 1585 (693-3564) |
| 52 | 4-SO₂Me | >10,000 | N.D. |
| 53 | 2-OMe | 1624 ± 248 | N.D. |
| 54 | 3-OMe | 485 ± 93 | N.D. |
| 55 | 4-OMe | 228 ± 45 | 3724 (952-14554) |
| 56 | 3,4-diOMe | 1862 ± 319 | N.D. |
| 57 | 3,5-diOMe | 1073 ± 85 | N.D. |
| 58 | 4-OH | >10,000 | N.D. |
| 59 | 4-Me | 137 ± 21 | N.D. |
| 60 | 3-Me | 45 ± 9 | 4183 |
| 61 | 2-F | 174 ± 27 | N.D. |
| 62 | 3-F | 53 ± 9 | 1520 (961-2405) |
| 63 | 4-F | 108 ± 19 | 887 (512-1534) |
| 64 | 3,4-diF | 47 ± 5 | N.D. |
| 65 | 2,4,6-triF | 81 ± 9 | N.D. |
| 66 | 2,3,4,5,6- | 34 ± 7 | N.D. |
| | pentaF | | |
| 67 | 2-Cl | 237 ± 35 | N.D. |
| 68 | 3-Cl | 30 ± 4 | 561 (365-862) |
| 69 | 2,4-diCl | 174 ± 34 | N.D. |
| 70 | 2-Cl,6-F | 408 ± 66 | N.D. |
| 71 | 4-Br | 151 ± 20 | N.D. |
| 72 | 4-CN | 443 ± 53 | N.D. |
| 73 | 2-CF₃ | 143 ± 34 | N.D. |
| 74 | 3-CF₃ | 32 ± 7 | 766 (395-1486) |
| 75 | 4-CF₃ | 194 ± 38 | N.D. |

^{a}Values are the mean ± SEM of at least three independent experiments in duplicate.
^{b}Values are expressed as mean (95% confidence interval) from at least three independent experiments in duplicate.

TABLE 6
Allosteric Modulating Activities of Compounds 76-84 in the hCB1R
Calcium Mobilization and mCB1R [³⁵S]GTPγS Binding Assays.

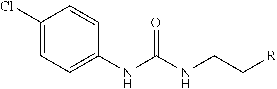

| | | | mCB1R |
|---|---|---|---|
| | | hCB1R | [³⁵S]GTPγS |
| | | Calcium assay | binding assay |
| Compound | Structure | IC₅₀ (nM)^{a} | IC₅₀ (nM)^{b} |
| 76 | 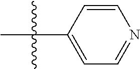 [SMILES: *c1ccncc1] | >10,000 | N.D. |
| 77 | 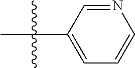 [SMILES: *c1cccnc1] | >10,000 | N.D. |
| 78 | 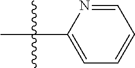 [SMILES: *c1ccccn1] | 3627 ± 687 | N.D. |
| 79 | 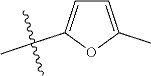 [SMILES: *c1ccc(C)o1] | 255 ± 40 | 2775 (1555-4953) |
| 80 | 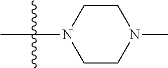 [SMILES: *N1CCN(C)CC1] | >10,000 | N.D. |
| 81 | 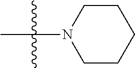 [SMILES: *N1CCCCC1] | >10,000 | N.D. |
| 82 | 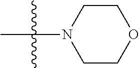 [SMILES: *N1CCOCC1] | >10,000 | N.D. |
| 83 | 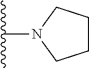 [SMILES: *N1CCCC1] | >10,000 | N.D. |
| 84 | NHCOMe | 1827 ± 362 | N.D. |

^{a}Values are the mean ± SEM of at least three independent experiments in duplicate.
^{b}Values are expressed as mean (95% confidence interval) from at least three independent experiments in duplicate.

CB1R has been demonstrated to possess constitutive activities which are needed maintain normal physiological functions. SR₁₄₁₇₁₆ acts as an CB1R inverse agonist, reducing the CB1R signaling on its own. It has been suggested that inhibition of this basal activity results in the adverse effects of SR₁₄₁₇₁₆. Therefore, the intrinsic activities the presently disclosed compounds were studied in the absence of the CB1R agonist CP55,940. As shown in FIG. 2, SR₁₄₁₇₁₆ imparted significant inverse agonism with IC₅₀ of 2.8 nM. Compound 2 reached the same level of inverse agonism produced by SR141716 only at the highest concentration of 10 (IC₅₀=1.47 μM). At 10 μM, compounds 9, 11, 14, and 35 only exhibited some degree of inverse agonism. In particular, thiophene analogue 11 produced very little inverse agonism up to 10 μM. These results show that the CB1R allosteric modulators had less liability of imparting adverse effects of SR_{141716.}

### Example 3

### Stability, Solubility, Permeability and Pharmacokinetic Studies

Metabolic stability assessment: Compounds were incubated with rat liver microsomes at 37° C. for a total of 45 minutes. The reaction was performed at pH 7.4 in 100 mM potassium phosphate buffer containing 0.5 mg/mL of rat liver microsomal protein. Phase I metabolism was assessed by adding NADPH to a final concentration of 1 mM and collecting samples at time points 0, 5, 15, 30 and 45 minutes. All collected samples were quenched 1:1 with ice-cold stop solution (1 μM labetalol and 1 μM glyburide in acetonitrile) and centrifuged to remove precipitated protein. Resulting supernatants were further diluted 1:4 with acetonitrile:water (1:1). Samples were analyzed by LC/MS/MS and calculations for half-life, and in-vitro clearance were accomplished using Microsoft Excel (2007).

Kinetic solubility assessment: A 10 pL of test compound stock solution (20 mM DMSO) was combined with 490 pL of phosphate buffer solution to reach a targeted concentration of 400 μM. The solution was agitated on a VX-2500 multi-tube vortexer (VWR International, Radnor, Pa., United States of America) for 2 hours at room temperature. Following agitation, the sample was filtrated on a glass-fiber filter (1 μm) and the eluate was diluted 400-fold with a mixture of acetonitrile: water (1:1). On each experimental occasion, nicardipine and imipramine were assessed as reference compounds for low and high solubility, respectively. All samples were assessed in triplicate and analyzed by LC-MS/MS using electrospray ionization against standards prepared in the same matrix.

Results: In an effort to advance CB1R allosteric modulators for therapeutics development, preliminary ADME assessment of some of the presently disclosed compounds was performed. Compound 11 (T1/2 =65 min) showed better metabolic stability than 2 (T1/2 =13 min). See Table 7, below. Solubility is another parameter to predict compound absorption and generally reflects bioavailability (see Kerns et al., Curr. Drug Metab. 2008, 9, 879-885), although it can be mitigated by formulation. As shown in Table 7, below, compound 11 (solubility=1.5 μM) had improved solubility compared to compound 2 (solubility <0.5 μM). Without being bound to any one theory, the improved solubility can be attributed to the looser packing of the five membered ring thiophene compared to the phenyl ring. Although 68 had a limited metabolic stability against rat liver microsomes (T_{1/2}=9.6 min) in vitro, it demonstrated excellent blood-brain permeability in the MDCK-MDR₁ Transwell assay with Papp values for both directions more than 15×10 ⁻⁶ cm/s, a cutoff value that is generally considered as CNS passive permeation.²³ It is not a P-glycoprotein substrate (efflux ratio BA/AB <2.5). The in vitro PK data corroborated with the in vitro pharmacokinetics study demonstrating that 68 is highly brain-penetrant with brain concentration is approximately twice of plasma concentration (Kp=2.01, FIG. 4). It reached peak levels both in plasma and brain at 30 min after i.p. administration with Cₘₐₓ values of 220.6 and 546 ng/mL in plasma and brain respectively.

Permeability Assessment. Bidirectional MDCK-MDR₁ permeability assay was performed by Paraza Pharma Inc. (Montreal, Canada). MDCK-mdr1 cells at passage 5 were seeded onto permeable polycarbonate supports in 12-well Costar Transwell plates and allowed to grow and differentiate for 3 days. On day 3, culture medium (DMEM supplemented with 10% FBS) was removed from both sides of the transwell inserts and cells were rinsed with warm HBSS. After the rinse step, the chambers were filled with warm transport buffer (HBSS containing 10 mM HEPES, 0.25% BSA, pH 7.4) and the plates were incubated at 37° C. for 30 min prior to TEER (Trans Epithelial Electric Resistance) measurements.

The buffer in the donor chamber (apical side for A-to-B assay, basolateral side for B-to-A assay) was removed and replaced with the working solution (10 μM test article in transport buffer). The plates were then placed at 37° C. under light agitation. At designated time points (30, 60 and 90 min), an aliquot of transport buffer from the receiver chamber was removed and replenished with fresh transport buffer. Samples were quenched with ice-cold ACN containing internal standard and then centrifuged to pellet protein. Resulting supernatants are further diluted with 50/50 ACN/H2O (H2O only for Atenolol) and submitted for LC-MS/MS analysis. Reported apparent permeability (Papp) values were calculated from single determination.

Atenolol and propranolol were tested as low and moderate permeability references. Bidirectional transport of digoxin was assessed to demonstrate Pgp activity/expression.

The apparent permeability (Papp, measured in cm/s) of a compound is determined according to the following formula from two indendepent experiments in duplicate:

$$Papp = \frac{( dQ ) / ( dt )}{A * C i * 60} ,$$

where dQ/dt is the net rate of appearance in the receiver compartment; A is the area of the Transwell measured in cm² (1.12 cm²); Ci is the initial concentration of compound added to the donor chamber; and 60 is the conversion factor for minute to second.

Pharmacokinetic Assessment. An in vivo pharmacokinetic assay was performed by Paraza Pharma Inc. (Montreal, Canada). On the morning of the PK study, male Sprague-Dawley rats weighing 258-277 g were dosed with either vehicle (5% Cremorphor, 5% ethanol in saline) or 14014-149 (10 mg/kg, i.p.). At selected timepoints (0.25, 0.5, 1, 3, 5, 8 and 24 hours post dose), 2 rats were anesthetized with isoflurane gas to perform a cardiac puncture to collect blood (for plasma analysis), followed by whole body perfusion with phosphate saline buffer (PBS, pH 7.4) to wash out any remaining blood from the organs. Brains were harvested and homogenized by mechanical sheering with a polytron with 1:4 (w/v) 25% isopropanol in water. Brain homogenates were extracted for drug quantification by LC-MS/MS.

TABLE 7
Metabolic stability of compounds in rat liver microsomes and their kinetic solubility.

| | Half-life | Clearance | Pₐₚₚ A to B | Pₐₚₚ B to A | Efflux | Solubility |
|---|---|---|---|---|---|---|
| Cmpd | (min)^{a} | (μL/min/mg)^{a} | (×10⁻⁶ cm/s)^{b} | (×10⁻⁶ cm/s)^{b} | BA/AB | (μM)^{b} |
| 2 | 13.4 ± 4.1 | 113.7 ± 34.4 | 2.6 ± 0 | 2.2 ± 0.1 | 0.8 | <0.5 |
| 11 | 65 ± 19.1 | 22.2 ± 6.5 | 1.6 ± 0.1 | 1.1 ± 0.4 | 0.7 | 1.5 ± 0.1 |
| 20 | 72 ± 6^{c} | 19.3 ± 1.6 | N.D. | N.D. | N.D. | <0.5 |
| 68 | 9.6 ± 0.6 | 144.1 ± 8.4 | 19.2 ± 0.4 | 21.3 ± 1.1 | 1.1 | <0.5 |

^{a}Values are expressed as mean ± SD from two independent experiments.
^{b}Values are expressed as mean ± SD from three independent experiments.
^{c}Percent parent compound remaining dropped to approximately 70% after 15 minutes but remains stable for the rest of the incubation (45 min).
N.D. Not determined

### Example 4

### Reinstatement Of Extinguished Cocaine-Seeking Behavior

Adult male Sprague-Dawley rats (Harlan, Indianapolis, Ind., United States of America) weighing 280-300 g were used in the study. Animals were housed individually on a 12/12 hr light/dark cycle (behavioral experiments were conducted during the light period) with free access to water and food except during experimental sessions.

The reinstatement procedure has been previously described. See Jing et al., Drug Alcohol Depend. 2014, 143, 251-256; and Thorn et al., Neuropsychopharmacology 2014, 39, 2309-2316. Briefly, rats were surgically implanted with a chronic indwelling jugular catheter. After one-week recovery, rats were trained to press the active lever (left lever) for infusion of cocaine (0.75 mg/kg/inf) under a fixed ratio [FR] schedule (starting FR =1, which was increased to FR 5 within 5 training sessions) schedule during daily 2-hr sessions for 14 days. Reinforcer deliveries were accompanied by the presentation of a stimulus light over the active lever followed by a 30-s time-out period during which lever presses had no programmed consequence. Following acquisition of cocaine self-administration, extinction of drug-seeking behavior took place during 2-hr daily sessions in which lever pressing produced no consequence. All other conditions remained unchanged. After 7 days of extinction, all rats reached the extinction criteria (total responses less than 20% of the training sessions).

Drug-induced reinstatement test was conducted on the day following the last extinction session. Rats were pretreated with vehicle, compounds 2 (15, 30 mg/kg) or 34 (10 mg/kg) 10 min prior to a priming injection of cocaine (10 mg/kg, i.p.) administered immediately before the start of the reinstatement session.

Data analyses: Data are expressed as mean ±S.E.M. Differences in active lever responding between the last extinction session and reinstatement session were determined with paired t tests (within subjects comparison). The effects of compounds 2 on reinstatement were analyzed by a one-way analysis of variance (ANOVA) followed by post hoc Bonferroni's test (between subjects comparison). The effects of compounds 34 on reinstatement was analyzed by Student's t test. P <0.05 was considered statistically significant.

Results: Blockade of the CB1 receptor in vivo with the antagonist/inverse agonist SR₁₄₁₇₁₆A has been demonstrated to reduce intake of palatable food, self-administration of several drugs of abuse, and reinstatement of food and drug-seeking behaviors. Rats pretreated with 1 and 2 have been previously shown to be less likely to seek drugs of abuse, such as cocaine or methamphetamine after a period of extinction. Therefore, two of the presently disclosed compounds, i.e., compounds 11 and 68, were studied to determine if they achieve the same effects in vivo.

As shown in FIG. 3A, a cocaine prime significantly reinstated the extinguished active lever response (t test: t[7] =16.29, p<0.0001).

Pretreatment with 68 (10 mg/kg, i.p.), but not 11 (10 mg/kg, i.p.), attenuated cocaine-induced reinstatement of cocaine-seeking behavior. Intriguingly, at this dose of 10 mg/kg i.p. compound 68 did not affect locomotion, in contrast to compound 11, which exhibited a significant reduction of locomotion 5 min after administration. See FIG. 3B.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A compound having a structure of Formula (I): 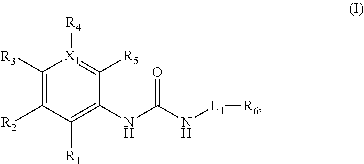
wherein:
X₁ is —C— or —N—;
each of R₁, R₂, R₃, and R₅ is independently selected from the group consisting of H, alkyl, substituted alkyl, halo, haloalkyl, alkoxy, nitro, and cyano, or wherein R₂ and R₃ together form an alkylene group;
R₄ is present or absent, and when present is selected from the group consisting of H, alkyl, substituted alkyl, halo, haloalkyl, alkoxy, nitro, and cyano;
L₁ is selected from the group consisting of alkylene, substituted alkylene, cycloalkylene, substituted cycloalkylene, heterocycloalkylene, substituted arylene, heteroarylene, and substituted heteroarylene; and
R₆ is selected from the group consisting of aryl, substituted aryl, heteroaryl, substituted heteroaryl, alkylamino, dialkylamino, acylamino, N-heterocycle, and substituted N-heterocycle;
or a pharmaceutically acceptable salt or solvate thereof.

2. The compound of claim 1, wherein X₁ is —C—.

3. The compound of claim 2, wherein R₁, R₂, R₄, and R₅ are each H, and the compound of Formula (I) has a structure of Formula (Ia): 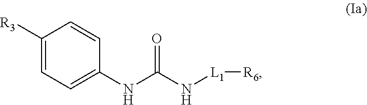
or a pharmaceutically acceptable salt or solvate thereof.

4. The compound of claim 3, wherein R₃ is Cl.

5. The compound of claim 3 or claim 4, wherein L₁ is selected from the group consisting of thiophenylene, pyridinylene, thiazolylene, alkylene, and substituted alkylene.

6. The compound of any one of claims 3-5, wherein R₆ is selected from the group consisting of phenyl, substituted phenyl, pyridinyl, furanyl, substituted furanyl, and -NHC(═O)CH_{3.}

7. The compound of any one of claims 3-6, wherein L₁ is thiophenylene and the compound has a structure of Formula (II): 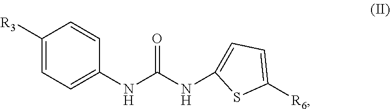
or a pharmaceutically acceptable salt or solvate thereof.

8. The compound of claim 7, wherein R₆ is selected from phenyl, substituted phenyl, or pyridinyl.

9. The compound of claim 7 or claim 8, wherein R₃ is Cl, R₆ is phenyl or substituted phenyl, and wherein the compound of Formula (II) has a structure of Formula (IIa): 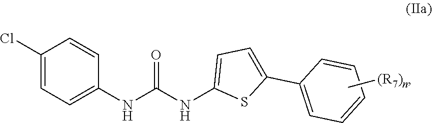
wherein:
n is 0, 1, 2, 3, 4, or 5; and
each R₇ is independently selected from the group consisting of halo, nitro, hydroxy, cyano, alkyl, aryl, acyl, ester, alkoxy, sulfonyl, and dialkylamino;
or a pharmaceutically acceptable salt or solvate thereof.

10. The compound of claim 9, wherein n is 1 or 2, and wherein each R₇ is halo, optionally chloro or fluoro.

11. The compound of claim 9, wherein n is 1 and R₇ is methoxy or methyl.

12. The compound of claim 9, wherein the compound is selected from the group consisting of:
1-(4-Chlorophenyl)-3-(5-phenylthiophen-2-yl)urea (11),
1-(4-Chlorophenyl)-3-[5-(4-fluorophenyl)thiophen-2-yl]urea (18),
1-(4-Chlorophenyl)-3-[5-(3-fluorophenyl)thiophen-2-yl]urea (19),
1-(4-Chlorophenyl)-3-[5-(2,4-difluorophenyl)thiophen-2-yl]urea (20),
1-(4-Chlorophenyl)-3-[5-(2-chlorophenyl)thiophen-2-yl]urea (21),
1-(4-Chlorophenyl)-3-[5-(3-chlorophenyl)thiophen-2-yl]urea (22),
1-(4-Chlorophenyl)-3-[5-(4-chlorophenyl)thiophen-2-yl]urea (23),
1-(4-Chlorophenyl)-3-[5-(3,4-dichlorophenyl)thiophen-2-yl]urea (24),
1-(4-Chlorophenyl)-3-[5-(3,5-dichlorophenyl)thiophen-2-yl]urea (25),
3-[5-(3-Acetylphenyl)thiophen-2-yl]-1-(4-chlorophenyl)urea (26),
Methyl 3-(5-{[(4-chlorophenyl)carbamoyl]amino}thiophen-2-yl)benzoate (27),
1-(4-Chlorophenyl)-3-[5-(3-methanesulfonylphenyl)thiophen-2-yl]urea (28),
1-(4-Chlorophenyl)-3-[5-(2-methoxyphenyl)thiophen-2-yl]urea (29),
1-(4-Chlorophenyl)-3-[5-(3-methoxyphenyl)thiophen-2-yl]urea (30),
1-(4-Chlorophenyl)-3-[5-(4-methoxyphenyl)thiophen-2-yl]urea (31),
1-(4-Chlorophenyl)-3-[5-(3-methylphenyl)thiophen-2-yl]urea (32),
1-(4-Chlorophenyl)-3-{5-[3-(dimethylamino)phenyl]thiophen-2-yl}urea (33),
1-(4-Chlorophenyl)-3[5-(pyridin-3-yl)thiophen-2-yl]urea (34), and
1-(4-Chlorophenyl)-3[5-(pyridin-4-yl)thiophen-2-yl]urea (35);
or a pharmaceutically acceptable salt or solvate thereof.

13. The compound of any one of claims 3-6, wherein L₁ is ethylene or substituted ethylene and the compound of Formula (Ia) has a structure of Formula (III): 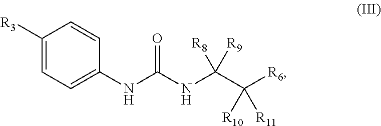
wherein:
each of R₈, R₉, R₁₀, and R₁₁ are independently selected from the group consisting of H, halo, and alkyl, or wherein two of R₈, R₉, R₁₀, and R₁₁ together from an alkylene group;
or a pharmaceutically acceptable salt or solvate thereof.

14. The compound of claim 13, wherein R₃ is chloro, each of R₈, R₉, R₁₀, and R₁₁ are H, R₆ is phenyl or substituted phenyl, and the compound of Formula (III) has a structure of Formula (IIIa): 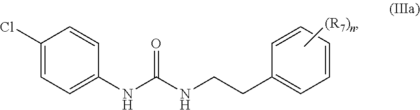
wherein:
n is 0, 1, 2, 3, 4, or 5; and
each R₇ is independently selected from the group consisting of halo, nitro, hydroxyl, cyano, alkyl, perfluoroalkyl, aryl, acyl, ester, alkoxyl, sulfonyl, and dialkylamino;
or a pharmaceutically acceptable salt or solvate thereof.

15. The compound of claim 14, wherein each R₇ is independently selected from the group consisting of fluoro, chloro, methyl, tert-butyl, phenyl, nitro, methoxy, dimethylamino, cyano, and trifluoromethyl.

16. The compound of claim 13, wherein the compound is selected from the group consisting of:
trans1-(4-Chlorophenyl)-3-(2-phenylcyclopropyl)urea (15),
cis1-(4-Chlorophenyl)-3-(2-phenylcyclopropyl)urea (16),
3-(4-Chlorophenyl)1-(2-phenylethyl)urea (44),
1-[2-(4-tert-Butylphenyl)ethyl]-3-(4-chlorophenyl)urea (45),
3-(4-Chlorophenyl)-1-[2-(4-phenylphenyl)ethyl]urea (46),
3-(4-Chlorophenyl)-1-[2-(4-chlorophenyl)ethyl]urea (47),
3-(4-Chlorophenyl)-1-[2-(4-nitrophenyl)ethyl]urea (48),
3-(4-Chlorophenyl)-1-[2-(4-hydroxy-3-methoxyphenyl)ethyl]urea (49),
3-(4-Chlorophenyl)1-{2-[3-(dimethylamino)phenyl]ethyl}urea (50),
3-(4-Chlorophenyl)1-{2-[4-(dimethylamino)phenyl]ethyl}urea (51),
3-(4-Chlorophenyl)-1-[2-(4-methanesulfonylphenyl)ethyl]urea (52),
3-(4-Chlorophenyl)-1-[2-(2-methoxyphenyl)ethyl]urea (53),
3-(4-Chlorophenyl)-1-[2-(3-methoxyphenyl)ethyl]urea (54),
3-(4-Chlorophenyl)-1-[2-(3-methoxyphenyl)ethyl]urea (55),
3-(4-Chlorophenyl)-1-[2-(3,4-dimethoxyphenyl)ethyl]urea (56),
3-(4-Chlorophenyl)-1-[2-(3,5-dimethoxyphenyl)ethyl]urea (57),
3-(4-Chlorophenyl)-1-[2-(4-hydroxyphenyl)ethyl]urea (58),
3-(4-Chlorophenyl)-1-[2-(4-methylphenyl)ethyl]urea (59),
3-(4-Chlorophenyl)-1-[2-(3-methylphenyl)ethyl]urea (60),
3-(4-Chlorophenyl)-1-[2-(2-fluorophenyl)ethyl]urea (61),
3-(4-Chlorophenyl)-1-[2-(3-fluorophenyl)ethyl]urea (62),
3-(4-Chlorophenyl)-1-[2-(4-fluorophenyl)ethyl]urea (63),
3-(4-Chlorophenyl)1-[2-(3,4-difluorophenyl)ethyl]urea (64),
3-(4-Chlorophenyl)1-[2-(2,4,6-trifluorophenyl)ethyl]urea (65),
3-(4-Chlorophenyl)1-[2-(2,3,4,5,6-pentafluorophenyl)ethyl]urea (66),
3-(4-Chlorophenyl)1-[2-(2-chlorophenyl)ethyl]urea (67),
3-(4-Chlorophenyl)1-[2-(3-chlorophenyl)ethyl]urea (68),
3-(4-Chlorophenyl)1-[2-(2,4-dichlorophenyl)ethyl]urea (69),
3-(4-Chlorophenyl)1-[2-(2-chloro-6-fluorophenyl)ethyl]urea (70),
3-(4-Chlorophenyl)1-[2-(4-bromophenyl)ethyl]urea (71),
3-(4-Chlorophenyl)1-[2-(4-cyanophenyl)ethyl]urea (72),
3-(4-Chlorophenyl)-1-{2-[2-(trifluoromethyl)phenyl]ethyl}urea (73),
3-(4-Chlorophenyl)-1-{2-[3-(trifluoromethyl)phenyl]ethyl}urea (74),
3-(4-Chlorophenyl)-1-{2-[4-(trifluoromethyl)phenyl]ethyl}urea (75),
3-(4-Chlorophenyl)1-[2-(pyridin-4-yl)ethyl]urea (76),
3-(4-Chlorophenyl)1-[2-(pyridin-3-yl)ethyl]urea (77)
3-(4-Chlorophenyl)1-[2-(pyridin-2-yl)ethyl]urea (78),
1-(4-Chlorophenyl)-3-[2-(5-methylfuran-2-yl)ethyl]urea (79),
3-(4-Chlorophenyl)-1-[2-(4-methylpiperazin-1-yl)ethyl]urea (80),
3-(4-Chlorophenyl)-1-[2-(piperidin-1-yl)ethyl]urea (81),
3-(4-Chlorophenyl)1-[2-(morpholin-4-yl)ethyl]urea (82),
1-(4-Chlorophenyl)-3-[2-(pyrrolidin-1-yl)ethyl]urea (83),
N-(2-{[(4-Chlorophenyl)carbamoyl]amino}ethyl)acetamide (84),
3-(4-Chlorophenyl)-1-(2-methyl-2-phenylpropyl)urea (38),
3-(4-Chlorophenyl)-1-(2,2-difluoro-2-phenylethyl)urea (39),
3-(4-Chlorophenyl)-1-(2-methyl1-phenylpropan-2-yl)urea (40),
1-(4-Chlorophenyl)-3-[(1-phenylcyclopropyl)methyl]urea (41), and
3-(1-Benzylcyclopropyl)-1-(4-chlorophenyl)urea (42);
or a pharmaceutically acceptable salt or solvate thereof.

17. The compound of claim 1, wherein the compound is selected from the group consisting of:
3-(4-Chlorophenyl)-1-{2-methoxy-5-[6-(pyrrolidin1-yl)pyridin-2-yl]phenyl}urea (6),
1-(4-Chlorophenyl)-3-(4-phenylpyridin-2-yl)urea (7),
1-(4-Chlorophenyl)-3-(6-phenylpyridin-2-yl)urea (8),
1-(4-Chlorophenyl)-3-(5-phenylpyridin-3-yl)urea (9),
1-(4-Chlorophenyl)-3-(2-phenylpyridin-4-yl)urea (10),
1-(4-Chlorophenyl)-3-(4-phenylthiophen-2-yl)urea (12),
1-(4-Chlorophenyl)-3-(5-phenylthiophen-3-yl)urea (13),
1-(4-Chlorophenyl)-3-(5-phenyl-1,3-thiazol-2-yl)urea (14),
3-(4-Chlorophenyl)-1-[(3R)-1-phenylpiperidin-3-yl]urea (17),
1-Benzyl-3-(4-chlorophenyl)urea (36),
3-(4-Chlorophenyl)-1-(3-phenylpropyl)urea (37), and
trans1-(4-Chlorophenyl)-3-[(2-phenylcyclopropyl)methyl]urea (43);
or a pharmaceutically acceptable salt or solvate thereof.

18. A pharmaceutical composition comprising a compound of any one of claims 1-17, and a pharmaceutically acceptable carrier.

19. A method of treating a cannabinoid 1 receptor (CB1R)-mediated disease or condition in a subject in need of treatment thereof, the method comprising administering to said subject a therapeutically effective amount of a compound of any one of claims 1-17 or a pharmaceutical composition of claim 18.

20. The method of claim 19, wherein the subject is a mammal, optionally a human.

21. The method of claim 19 or claim 20, wherein the disease or condition is selected from the group consisting of drug addiction, obesity, cancer, pain, female infertility, memory loss, congnitive dysfunction, Parkinson's disease, dyskinesia, tardive dyskinesia, Alzheimer's disease, amyotrophic lateral sclerosis (ALS), Tourette's Syndrome, stroke, atherosclerosis, hypotension, intestinal hypoactivity in paralytic ileus, inflammation, osteoporosis, hypercholesterolemia, hyslipidemia, diabetes, retinopathy, glaucoma, anxiety, depression and other mood disorders, gastrointestinal disorders, and metabolic disorders.

22. The method of claim 21, wherein the disease is obesity or drug addiction, optionally wherein the drug addiction is selected from cocaine addiction, opiod addiction, amphetamine addiction, cannabinoid addition, tobacco addiction, and alcohol addiction.

23. The method of any one of claims 19-22, wherein the compound is a compound of Formula (II): 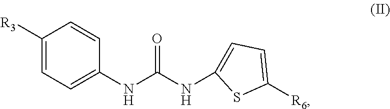
optionally wherein R₃ is chloro, further optionally wherein R₆ is substituted phenyl.

24. The method of any one of claims 19-22, wherein the compound is a compound of Formula (III): 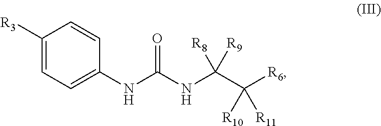
optionally wherein R₃ is chloro, further optionally wherein each of Rs, R₉, R₁₀, and R₁₁ is H.

25. The method of any one of claims 19-22, wherein the compound is selected from the group consisting of:
1-(4-Chlorophenyl)-3-(5-phenylthiophen-2-yl)urea (11),
1-(4-Chlorophenyl)-3-[5-(2,4-difluorophenyl)thiophen-2-yl]urea (20),
1-(4-Chlorophenyl)-3-[5-(2-chlorophenyl)thiophen-2-yl]urea (21),
1-(4-Chlorophenyl)-3-[5-(4-methoxyphenyl)thiophen-2-yl]urea (31),
3-(4-Chlorophenyl)-1-[2-(3-chlorophenyl)ethyl]urea (68), and
3-(4-Chlorophenyl)-1-{2-[3-(trifluoromethyl)phenyl]ethyl}urea (74);
or a pharmaceutically acceptable salt or solvate thereof.

26. A method of treating obesity in a subject in need of treatment thereof, the method comprising administering to said subject a therapeutically effective amount of a compound of any one of claims 1-17 or a pharmaceutical composition of claim 18.

27. The method of claim 26, wherein the compound is selected from the group consisting of:
1-(4-Chlorophenyl)-3-(5-phenylthiophen-2-yl)urea (11),
1-(4-Chlorophenyl)-3-[5-(2,4-difluorophenyl)thiophen-2-yl]urea (20),
1-(4-Chlorophenyl)-3-[5-(2-chlorophenyl)thiophen-2-yl]urea (21),
1-(4-Chlorophenyl)-3-[5-(4-methoxyphenyl)thiophen-2-yl]urea (31),
3-(4-Chlorophenyl)-1-[2-(3-chlorophenyl)ethyl]urea (68), and
3-(4-Chlorophenyl)-1-{2-[3-(trifluoromethyl)phenyl]ethyl}urea (74);
or a pharmaceutically acceptable salt or solvate thereof.

28. A method for preventing or inhibiting substance abuse and/or addiction, an addictive behavior, or of a symptom, behavior, or condition associated with substance abuse and/or addiction, the method comprising administering to a subject in need thereof a therapeutically effective amount of a compound of any one of claims 1-17 or a pharmaceutical composition of claim 18.

29. The method of claim 28, wheiren the substance abuse and/or addiction is selected from cocaine addiction, opiod addiction, amphetamine addiction, cannabinoid addition, tobacco addiction, and alcohol addiction.

30. The method of claim 28 or claim 29, wherein the administration prevents or inhibits relapse.

31. The method of any one of claims 28-30, wherein the compound is selected from the group consisting of:
1-(4-Chlorophenyl)-3-(5-phenylthiophen-2-yl)urea (11),
1-(4-Chlorophenyl)-3-[5-(2,4-difluorophenyl)thiophen-2-yl]urea (20),
1-(4-Chlorophenyl)-3-[5-(2-chlorophenyl)thiophen-2-yl]urea (21),
1-(4-Chlorophenyl)-3-[5-(4-methoxyphenyl)thiophen-2-yl]urea (31),
3-(4-Chlorophenyl)-1-[2-(3-chlorophenyl)ethyl]urea (68), and
3-(4-Chlorophenyl)-1-{2-[3-(trifluoromethyl)phenyl]ethyl}urea (74);
or a pharmaceutically acceptable salt or solvate thereof.

32. A method of modulating the activity of cannabinoid 1 receptor (CB1R), wherein the method comprises contacting a sample comprising CB1R with a compound of one of claims 1-17 or a pharmaceutical composition of claim 18.
